(19) United States
(12) Patent Application Publication
(10) Pub. No.: US 2022/0298578 A1
(43) Pub. Date: Sep. 22, 2022

(54) DETECTION OF RELB ACTIVATION FOR PREDICTING A PROGNOSTIC IN B-CELL LYMPHOMA

(71) Applicants: UNIVERSITÉ DE PARIS, Paris (FR); INSTITUT NATIONAL DE LA SANTE ET DE LA RECHERCHE MEDICALE, Paris (FR)

(72) Inventor: Véronique BAUD, PARIS (FR)

(21) Appl. No.: 17/596,758

(22) PCT Filed: Jun. 25, 2020
(86) PCT No.: PCT/EP2020/067814

§ 371 Date: Dec. 17, 2021

(30) Foreign Application Priority Data
Jun. 25, 2019 (EP) 19305847.6

(51) Int. Cl. C12Q 1/6886

(52) U.S. Cl. CPC C12Q 1/6886; C12Q 2600/112; C12Q 2600/158

(57) ABSTRACT

The invention relates to a method for predicting the prognosis of a patient suffering from a B-cell lymphoma, through the detection of the status of activation of the RelB protein, in a biological sample of said patient. The inventors indeed identified an associated gene expression signature in the biological sample. Some genes from said signature are over-expressed and other are under-expressed and allow detecting RelB activation and predicting a worse prognosis in B-cell lymphoma.

### FILED OF THE INVENTION

The present invention relates to a method for predicting the prognosis of a patient suffering from a B-cell lymphoma, through the detection of the status of activation of the RelB protein, in a biological sample of said patient. The inventors indeed identified an associated gene expression signature in the biological sample. Some genes from said signature are over-expressed and other are under-expressed and allow detecting RelB activation and predicting a worse prognosis in B-cell lymphoma.

### BACKGROUND OF THE INVENTION

NF-κB transcription factors family plays a crucial role in the inflammatory and immune response, cell proliferation and survival. In mammals, the NF-κB family is composed of five members, RelA (p65), RelB, cRel (Rel), NF-κB1 (p50 and its precursor p105) and NF-κB2 (p52 and its precursor p100). These proteins form various homo- and heterodimeric complexes, the activity of which is regulated by two main pathways. The first one, known as the canonical NF-κB activation pathway, mainly applies to RelA and/or cRel containing complexes. The second one, the alternative NF-κB activation pathway, leads to the activation of RelB containing dimers.

The B-cell lymphomas are types of lymphoma affecting B cells. Lymphomas are “blood cancers” in the lymph nodes. They develop more frequently in adults and in immunocompromised individuals. B-cell lymphomas include both Hodgkin's lymphomas and most non-Hodgkin lymphomas. They are typically divided into low and high grade, typically corresponding to indolent (slow growing) lymphomas and aggressive lymphomas, respectively. Prognosis and treatment depend on the specific type of lymphoma as well as the stage and grade. Treatment includes radiation and chemotherapy. Early-stage indolent B-cell lymphomas can often be treated with radiation alone, with long-term non-recurrence. Early-stage aggressive disease is treated with chemotherapy and often radiation, with a 70-90% cure rate. Late-stage indolent lymphomas are sometimes left untreated and monitored until they progress. Late-stage aggressive disease is treated with chemotherapy, with cure rates of over 70%.

Diffuse Large B-cell Lymphoma (DLBCL) is the most common of non-Hodgkin lymphoma in adults (Pileri S A, Harris N L, Jaffe E S, Cox J. WHO Classification of Tumours of Haematopoietic and Lymphoid Tissues. Revised 4t. IARC publications; 2017). The median age of incidence is the 7th decade of life, although it can occur in young adults and more rarely in children. Its most usual clinical presentation is one or multiple fast growing nodal and/or extra-nodal masses (up to 40% of cases), being the gastrointestinal tract the most frequent site. Even though cure rates have significantly improved since the introduction of anti-CD20 monoclonal antibody rituximab into conventional chemotherapy, refractory/relapse cases can reach up to 40% (De Leval L et al., 2010).

Gene expression profiles (GEP) allowed cases to be separated into two main subgroups that differed in prognosis, based on their cell of origin (COO): germinal center B-cell-like (GCB) and activated B-cell-like (ABC), this latter being associated with worse outcome (Alizadeh A A, et al., 2000). Even though this classification is largely used, heterogeneity within groups is acknowledged and a better stratification for patients is needed. WO2014/047422 discloses p100 and p105 dependent genes.

Surprisingly, the inventors discovered that the activation status of the RelB NF-κB subunit was a prognostic biomarker of patients suffering from a-B-cell Lymphoma, preferably DLBCL.

### SUMMARY OF THE INVENTION

Accordingly, the inventors surprisingly found that the activation status of the RelB NF-κB subunit was a prognostic biomarker of patients suffering from a-B-cell Lymphoma. In particular, the activation status of the RelB NF-κB subunit can be associated to a gene expression signature.

Thus, inventors identified such genes from said signature as good biomarkers for evaluating the prognosis of B-cell lymphoma patients, preferably Diffuse large B-cell lymphoma (DLBCL), Follicular lymphoma, Marginal zone B-cell lymphoma (MZL) or Mucosa-Associated Lymphatic Tissue lymphoma (MALT), Small lymphocytic lymphoma (also known as chronic lymphocytic leukemia, CLL), Mantle cell lymphoma (MCL), DLBCL variants or sub-types such as Primary mediastinal (thymic) large B cell lymphoma, T cell/histiocyte-rich large B-cell lymphoma, Primary cutaneous diffuse large B-cell lymphoma, leg type (Primary cutaneous DLBCL, leg type), EBV positive diffuse large B-cell lymphoma of the elderly, Diffuse large B-cell lymphoma associated with inflammation, Burkitt's lymphoma, Lymphoplasmacytic lymphoma, which may manifest as Waldenström's macroglobulinemia, Nodal marginal zone B cell lymphoma (NMZL), Splenic marginal zone lymphoma (SMZL), Intravascular large B-cell lymphoma, Primary effusion lymphoma, Lymphomatoid granulomatosis, Primary central nervous system lymphoma, ALK-positive large B-cell lymphoma, Plasmablastic lymphoma, Large B-cell lymphoma arising in HHV8-associated multicentric Castleman's disease, B-cell lymphoma, unclassifiable with features intermediate between diffuse large B-cell lymphoma and Burkitt lymphoma, B-cell lymphoma, unclassifiable with features intermediate between diffuse large B-cell lymphoma and classical Hodgkin lymphoma.

In a more preferred embodiment, subject suffers from diffuse large B-cell lymphoma (DLBCL).

In a first aspect, the invention relates to an in vitro method for predicting the prognosis of a subject suffering from a B-cell lymphoma, said method comprising the step of detecting the status of DNA-binding activation of the RelB protein, in a biological sample of said patient. The method according to the invention relates the identification by the inventor of the genetic signature associated to the DNA-binding activation of the RelB protein, detected by measuring the expression level of activated-RelB dependent genes from an expression signature in the biological sample of said subject. In particular, expression of genes listed in Table 2 was identified as a worse prognosis for DLBCL patients.

In a preferred embodiment, the method comprising determining the expression level of at least one gene selected in the group consisting of SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, RAP1A gene, SNX20 gene, SEMA4D gene, ZNF224 gene, RNASEL gene, ARSD gene, TTC39B gene, ZNF81 gene, ATP5S gene, ZNF818P gene, ZNF829 gene, RGS3 gene, CECR7 gene, ZNF667-AS1 gene, ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SLC25A33 gene, MFSD14C gene, C12orf73 gene, DNLZ gene, PTEN gene, DENR gene, MTFMT gene, CSKMT gene, ARHGAP30 gene, gene comprising in its Coding DNA Sequence (CDS) SEQ ID NO:3, gene comprising in its CDS, gene comprising in its CDS SEQ ID NO:47, gene comprising in its CDS SEQ ID NO:129, gene comprising in its CDS SEQ ID NO:135, gene comprising in its CDS SEQ ID NO:136 and gene comprising in its CDS SEQ ID NO:139.

In a second aspect, the invention relates to an in vitro method for monitoring the evolution of B-cell lymphoma in a subject being diagnosed for B-cell lymphoma, said method comprising:
a) determining the status of DNA-binding activation of the RelB protein, in a biological sample of said subject, at a first time point,
b) determining the status of DNA-binding activation of the RelB protein, in a biological sample of said subject, at a second time point, and
c) comparing the status of DNA-binding activation of the RelB protein determined in step b) to the status of DNA-binding activation of the RelB protein determined in step a).

In a third aspect, the invention relates to a method for determining or adapting a therapeutic regimen suitable for a subject diagnosed for B-cell lymphoma comprising the step of:
a. determining the status of DNA-binding activation of the RelB protein, in a biological sample of a subject prior to administration of treatment or during treatment of said subject,
b. determining the status of DNA-binding activation of the RelB protein, in a biological sample of the subject after administration of treatment of said subject,
c. comparing the status of DNA-binding activation determined in step b) to the status of DNA-binding activation determined in step a),
d. adapting/modifying the therapeutic regimen for the subject based on the comparison of step c).

In a preferred embodiment, the status of DNA-binding activation of the RelB protein is determined by the determination of the level of expression of at least one gene of the signature as listed in Table 2, preferably of at least five of said signature, preferably of at least ten of said signature and even more preferably all the genes of said signature.

In a preferred embodiment, the biological sample of use in the method of the invention is body effluent such as, for example, urine or blood sample or a tumor sample such as, for example biopsy or surgical/resected specimen.

In another embodiment, the at least one RelB-dependent gene expression signature of the methods according to the invention is determined by RNAseq, microarray, Nanostring or RT-LMPA.

In a preferred embodiment, the subject according to the invention is suffering from Diffuse Large B-cell Lymphoma (DLBCL).

The invention relates also to the use of a kit for predicting a prognosis of patients suffering from B-cell lymphoma, comprising primers or approaches targeting or evaluating specifically the gene sequence and/or expression of SEQ ID NO:1-140.

As used herein, the term “approaches” refers to techniques described below for detecting expression of genes such as, for example RNAseq, DNA microarray, Nanostring or RT-MLPA.

The invention is particularly suited to predict a worse prognosis for patients suffering from DLBCL. As shown in the experimental part, NF-κB signature does not reflect the status of RelB activation and accordingly, the classification of the background art is not enough to correctly stratify the DLBCL patients. It is known that activated B-cell-like (ABC) subtype was associated with worse outcome for DLBCL, but many patients classified in germinal center B-cell-like (GCB) subtype associated with good outcome had finally a worse outcome. Thus, it is admitted that DLBCL patients are really difficult to correctly stratify due to the heterogeneity of the disease. By determining the gene expression profile which is actually linked to the activation status of RelB (i.e. when RelB is found associated with DNA and not only when it is located in the nucleus) inventor has been able to determine a valuable tool to prognose the fate of subjects suffering from B lymphomas, more particularly patients suffering from DLBCL. The signature of the invention is not linked to previously described NF-κB/RelB signature, indicating that said NF-κB/RelB previously described signatures does not reflect the actual DNA binding activity status of RelB which is surprisingly found by the inventor as particularly relevant in regard with prognosis of B cell lymphoma.

### LEGEND OF DRAWING

FIG. 1. Prognostic impact of RelB activation on the GHEDI cohort of DLBCL patients. (A) Kaplan Meier survival curves for patients with RelB activation status defined by EMSA (n=66). The estimated probability of overall survival in 2 years is 75.4% (SE 0.054), and in 5 years 65.1% (SE 0.074). The estimated probability of overall survival for RelB negative and positive patients in 2 years is of 95.5% (SE 0.044) and 65.5% (SE0.072), respectively; and for 5 years, 81.1% (SE 0.104) and 57.3% (SE 0.099), respectively. The mean survival for RelB negative and positive patients is 61.9 (CI 95%: 53.4-70.3) and 44.6 (CI 95%: 37-52.1) months, respectively. (B) Survival curves for patients with RelB activation status defined by EMSA adjusted by grouped IPI by multivariate Cox regression (n=66). (C) Kaplan Meier survival curves for patients with RelB status defined by EMSA, including only R-CHOP treated patients (n=40). The estimated probability of overall survival in 2 years is 72.2% (SE 0.071), and in 5 years 57.8% (SE 0.088). The estimated probability of overall survival for RelB negative and positive patients in 2 years is of 100% and 59.3% (SE0.095), respectively; and for 5 years, 79.5% (SE 0.131) and 47.6% (SE 0.110), respectively. The mean survival for RelB negative and positive patients is 63.1 (CI 95%: 54.3-71.9) and 39 (CI 95%: 29-49) months, respectively. (D) Survival curves for patients with RelB activation status defined by EMSA, including only R-CHOP treated patients, adjusted by grouped IPI by multivariate Cox regression (n=40). (E) Kaplan Meier survival curves for patients with RelB activation status defined by RelB signature (n=61). The estimated probability of overall survival in 2 years is 80.1% (SE 0.051), and in 5 years 67.1% (SE 0.079). The estimated probability of overall survival for RelB negative and positive patients in 2 years is of 92.6% (SE 0.05) and 70.5% (SE0.078), respectively; and for 5 years, 79.4% (SE 0.102) and 57.7% (SE 0.113), respectively. The mean survival for RelB negative and positive patients is 60.3 (CI 95%: 51.8-68.8) and 45.4 (CI 95%: 37-53.8) months, respectively. (F) Survival curves for patients with RelB activation status defined by RelB signature adjusted by grouped IPI by multivariate Cox regression (n=61). (G) Kaplan Meier survival curves for patients with RelB status defined by RelB signature, including only R-CHOP treated patients (n=37). The estimated probability of overall survival in 2 years is 78.2% (SE 0.068), and in 5 years 62.7% (SE 0.091). The estimated probability of overall survival for RelB negative and positive patients in 2 years is of 94.1% (SE 0.057) and 65% (SE0.107), respectively; and for 5 years, 77.7% (SE 0.118) and 50% (SE 0.129), respectively. The mean survival for RelB negative and positive patients is 60.3 (CI 95%: 49.9-70.7) and 41.1 (CI 95%: 29.7-52.5) months, respectively. (H) Survival curves for patients with RelB activation status defined by RelB signature, including only R-CHOP treated patients, adjusted by grouped IPI by multivariate Cox regression (n=37). (I) Kaplan Meier survival curves for patients with RelB status defined by RelB signature, including only R-CHOP treated patients (n=102). The estimated probability of overall survival in 2 years is 74% (SE 0.045), and in 5 years 56.6% (SE 0.059). The estimated probability of overall survival for RelB negative and positive patients in 2 years is of 80.1% (SE 0.056) and 67.4% (SE0.069), respectively; and for 5 years, 65% (SE 0.073) and 43.5% (SE 0.102), respectively. The mean survival for RelB negative and positive patients is 59 (CI 95%: 50.4-67.6) and 41.4 (CI 95%: 34.1-48.7) months, respectively. (J) Survival curves for patients with RelB activation status defined by RelB signature, including only R-CHOP treated patients, adjusted by grouped IPI and COO classification by multivariate Cox regression (n=102).

### DETAILED DESCRIPTION OF THE INVENTION

As intended herein, the term “comprising” has the meaning of “including” or “containing”, which means that when an object “comprises” one or several elements, other elements than those mentioned may also be included in the object. In contrast, when an object is said to “consist of” one or several elements, the object cannot include other elements than those mentioned.

To our knowledge, no study has ever revealed that the status of activation of the RelB protein was suitable to predict the prognostic of B-cell Lymphoma patients, more particularly DLBCL patients and associated with the detection of a gene expression signature in the biological sample of said subject, which was not known before either.

Yet, inventor has observed that RelB is frequently activated in both GCB and ABC DLBCL subtypes. Inventor has assessed the activation status of classical (RelA and cRel) and alternative (RelB) NF-κB subunits in 66 de novo DLBCL patients from the GHEDI cohort from the Lymphoma Study Association (LYSA) national cooperator group. The characteristics of this group are summarized below on Table 1 below.

TABLE 1
Characterization of the patients from the GHEDI cohort.

| | RelB | | | | RelB | | | | RelB | | | |
|---|---|---|---|---|---|---|---|---|---|---|---|---|
| | EMSA - Total cases | | | | EMSA - R-CHOP | | | | Signature - Total cases | | | |
| | Positive | | Negative | | Positive | | Negative | | Positive | | Negative | |
| Characteristics | n(%) | | n(%) | | n(%) | | n(%) | | n(%) | | n(%) | |
| Age > 60 years | 23 | (52.3) | 9 | (40.9) | 22 | (81.5) | 8 | (61.5) | 16 | (45) | 13 | (48.1) |
| Sex (F) | 25 | (56.8) | 12 | (54.5) | 14 | (51.9) | 6 | (46.2) | 19 | (55.9) | 15 | (55.6) |
| COO classification | | | | | | | | | | | | |
| GCB | 15 | (34.1) | 9 | (40.9) | 6 | (22.2) | 4 | (30.8) | 13 | (38.2) | 9 | (33.3) |
| ABC | 28 | (63.6) | 13 | (59.1) | 20 | (74.1) | 9 | (69.2) | 20 | (58.8) | 18 | (66.7) |
| Unclassified | 1 | (2.3) | 0 | (0) | 1 | (3.7) | 0 | (0) | 1 | (2.9) | 0 | (0) |
| ECOG > 1 | 14 | (31.8) | 4 | (18.2) | 12 | (44.4) | 2 | (15.4) | 9 | (26.5) | 7 | (25.9) |
| Extranodal site ≥ 2 | 20 | (45.5) | 5 | (22.7) | 15 | (55.6) | 3 | (23.1) | 15 | (44.1) | 8 | (29.6) |
| LDH > ULN | 29 | (67.4) | 15 | (68.2) | 20 | (74.1) | 10 | (76.9) | 21 | (63.6) | 19 | (70.4) |
| IPI score | | | | | | | | | | | | |
| 0 | 3 | (6.8) | 3 | (13.6) | 0 | (0) | 0 | (0) | 3 | (8.8) | 2 | (7.4) |
| 1 | 10 | (22.7) | 5 | (22.7) | 3 | (11.1) | 4 | (30.8) | 9 | (26.5) | 6 | (22.2) |
| 2 | 5 | (11.4) | 4 | (18.2) | 4 | (14.8) | 2 | (15.4) | 4 | (11.8) | 4 | (14.8) |
| 3 | 10 | (22.7) | 6 | (27.3) | 6 | (22.2) | 4 | (30.8) | 6 | (17.6) | 9 | (33.3) |
| 4 | 8 | (18.2) | 4 | (18.2) | 6 | (22.2) | 3 | (23.1) | 8 | (23.5) | 4 | (14.8) |
| 5 | 8 | (18.2) | 0 | (0) | 8 | (29.6) | 0 | (0) | 4 | (11.8) | 2 | (7.4) |
| Grouped IPI score | | | | | | | | | | | | |
| High risk (3-5) | 26 | 59.1) | 10 | (45.5) | 20 | (74.1) | 7 | (53.8) | 18 | (52.9) | 15 | (55.6) |
| Total | 44 | (66.7) | 22 | (33.3) | 27 | (67.5) | 13 | (32.5) | 34 | (55.7) | 27 | (44.3) |

| | RelB | | | | RelB | | | |
|---|---|---|---|---|---|---|---|---|
| | Signature - | | | | Signature | | | |
| | R-CHOP | | | | R-CHOP/GCB/ABC | | | |
| | Positive | | Negative | | Positive | | Negative | |
| Characteristics | n(%) | | n(%) | | n(%) | | n(%) | |
| Age > 60 years | 15 | (75) | 12 | (70.6) | 37 | (80.4) | 46 | (88.5) |
| Sex (F) | 10 | (50) | 8 | (47.1) | 21 | (45.7) | 28 | (53.8) |
| COO classification | | | | | | | | |
| GCB | 6 | (30) | 4 | (23.5) | 23 | (50) | 19 | (36.5) |
| ABC | 13 | (65) | 13 | (76.5) | 23 | (50) | 33 | (63.5) |
| Unclassified | 1 | (5) | 0 | (0) | 0 | (0) | 0 | (0) |
| ECOG > 1 | 7 | (35) | 5 | (29.4) | 13 | (28.3) | 13 | (25) |
| Extranodal site ≥ 2 | 10 | (50) | 6 | (35.3) | 19 | (41.3) | 19 | (36.5) |
| LDH > ULN | 15 | (75) | 12 | (70.6) | 34 | (73.9) | 41 | (78.8) |
| IPI score | | | | | | | | |
| 0 | 0 | (0) | 0 | (0) | 1 | (2.2) | 0 | (0) |
| 1 | 3 | (15) | 4 | (23.5) | 5 | (10.9) | 4 | (7.7) |
| 2 | 4 | (20) | 2 | (11.8) | 10 | (21.7) | 9 | (17.3) |
| 3 | 3 | (15) | 6 | (35.3) | 10 | (21.7) | 20 | (38.5) |
| 4 | 6 | (30) | 3 | (17.6) | 13 | (28.3) | 14 | (26.9) |
| 5 | 4 | (20) | 2 | (11.8) | 7 | (15.2) | 5 | (9.6) |
| Grouped IPI score | | | | | | | | |
| High risk (3-5) | 13 | (65) | 11 | (64.7) | 30 | (65.2) | 39 | (75) |
| Total | 20 | (54) | 17 | (46) | 46 | (46.9) | 52 | (53.1) |

For analysis, patients who received R-CHOP 14, R-CHOP 21 and mini-R-CHOP were grouped in “R-CHOP”. Patients who received R-ACVBP+conso, ACVBP+ASCT and ACVBP were grouped in “R-ACVBP”. ECOG, Eastern Cooperative Oncology Group performance status; IPI, international prognostic index; LDH, lactate dehydrogenase; ULN, upper limit of normal; COO, cell of origin; GCB, germinal center B-cell; ABC, activated B-cell; R, rituximab; CHOP, cyclophosphamide, doxorubicin, vincristine, and prednisone; ACVBP, doxorubicin, cyclophosphamide, vindesine, bleomycin, prednisone; Conso, consolidation; ASCT, autologous stem cell transplantation.

As used herein, “R-CHOP” refers to a combination of Rituximab, Cyclophosphamide, Hydroxyadriamicyne, Oncovin (vincristine) and Prednisone. As used herein, “R-ACVBP” refers to a combination of Rituximab, Adriamycine, Cyclophosphamide, Vindesine, Bleomycine and Prednisone.

In particular, RelB subunit presented a DNA binding activity in 44 cases (66.6%), well distributed among ABC and GCB subtype (Table 1). Further, inventors evaluated the effect of this RelB activation in DLBCL patients and have shown that RelB activation correlated with worse overall survival in said 66 de novo DLBCL patients (Table 1).

The status of activation of the RelB protein can be directly assessed by EMSA combined with supershift. As used herein, EMSA combined with supershift refers to a biochemical method allowing the detection of DNA binding of RelB containing complexes (Jacque et al., 2005). Whole cell extract was prepared and analyzed for DNA binding activity using the HIV-LTR tandem κB oligonucleotide as κB probe (Jacque et al., 2005). For supershift assays, whole cell extracts were incubated with specific antibodies for 30 min on ice before incubation with the labeled probe.

NF-κB (Nuclear Factor-KappaB) is a heterodimeric protein composed of different combinations of members of the Rel family of transcription factors, including NF-κB1 (p50), NF-κB2 (p52), RelA (p65), RelB, and c-Rel (Rel). Hetero and homo-dimerization of NF-κB proteins which exhibit differential binding specificities includes p50/RelA, p50/c-Rel, p52/c-Rel, p65/c-Rel, RelA/RelA, p50/p50, p52/p52, RelB/p50 and RelB/p52 and numerous other complexes.

NF-κB is known to be important in regulating a variety of cellular responses. It belongs to the category of “rapid-acting” primary transcription factors, i.e. transcription factors that are present in cells in an inactive state and do not require new protein synthesis to be activated. This allows NF-κB to be a first responder to harmful cellular stimuli. Known inducers of NF-κB activity are highly variable and include reactive oxygen species (ROS), tumor necrosis factor alpha (TNFα), interleukin 1-beta (IL-1β), bacterial lipopolysaccharides (LPS), Toll-like receptors (TLRs), lymphotoxin-α, lymphotoxin-β, BAFF, RANKL, isoproterenol, cocaine, virus, ionizing radiation, and genotoxic agents.

“RelB” or “transcription factor RelB” is a protein that in humans is encoded by the RELB gene and is accessible under the Uniprot number Q01201. “RelB gene” herein designates a protein coding gene. Its DNA sequence is located on chromosome 19 (45,001,449-45,038,194).

### Methods of the Invention

In a first aspect, the present invention therefore relates to an in vitro method for predicting the prognosis of a subject suffering from a B-cell lymphoma, said method comprising the step of detecting the status of activation of the RelB protein, in a biological sample of said patient.

As used herein, the term “subject” refers to a mammal, preferably a human. Preferably, it refers to a human patient that may be healthy (without any symptoms of B-cell lymphoma cancer), or that is thought to develop or is suspected of suffering from cancer, preferably B-cell lymphoma. Said subject for example presents at least one of the following symptoms: night sweats, fever, unexplained weight loss, fatigue, appetite loss, trouble breathing, pain or swelling in belly, severe itching. Said subject may also have suffered from a B-cell lymphoma cancer in the past, has been treated, and is monitored for potential disease recurrence. Said subject may also seem to be healthy but is predisposed to develop a B-cell lymphoma, for example genetic, because of, e.g., family history such as a member of his family is suffering or has suffered from the same disease. More preferably, in the methods according to the invention, subject is thought to develop or is suspected of suffering from diffuse large B-cell lymphoma (DLBCL). Diffuse large B-cell lymphoma is the most common subtype of non-Hodgkin lymphoma (NHL) in adults characterized by a median age of presentation in the sixth decade of life (but also rarely occurring in adolescents and children) with the initial presentation being single or multiple rapidly growing masses (that may or may not be painful) in nodal or extranodal sites (such as thyroid, skin, breast, gastrointestinal tract, testes, bone, or brain) and that can be accompanied by symptoms of fever, night sweats and weight loss. DLBCL has an aggressive disease course, with the elderly having a poorer prognosis than younger patients, and with relapses being common.

As used herein, the expression “biological sample” refers to solid tissues such as, for example, a tumor sample such as, for example biopsy or surgical/resected specimen or to fluids, body effluents such as, for example, blood, serum, plasma, urines, feces. Preferably, said biological sample is a fluid sample and most preferably a blood or urines.

The terms “worse prognosis”, “bad outcome”, “bad clinical outcome”, “worse overall survival” when related to a subject, in the context of the present invention, means a subject who is suffering from a B-cell lymphoma, more particularly a DLBCL, which is particularly aggressive or resistant to current therapies thereby resulting in a significant lowering of overall survival of said subjects, when compared to subjects from less aggressive B cell lymphoma. “Worse prognosis”, “bad outcome”, “bad clinical outcome”, is meant to include also worse Overall Survival (OS, defined as period of time for which patient is alive after disease diagnostic), worse Progression Free Survival (PFS, the length of time during and after the treatment of the disease, that a patient lives with the disease but it does not get worse), worse FFS=Failure Free Survival (FFS, defined as period of time with the absence of relapse, non-relapse mortality or addition of another systemic therapy) or worse Event Free Survival (EFS, the length of time after primary treatment for a cancer ends that the patient remains free of certain complications or events that the treatment was intended to prevent or delay).

In a preferred embodiment, the activation of the RelB protein is detected by measuring the expression level of at least one RelB-dependent gene from an expression signature associated with the activation status of RelB in a biological sample from said subject.

The terms “gene expression signature”, “gene signature”, “RelB activation signature” or “RelB signature”, when used herein, refer to the gene expression profile that has been identified by the inventor as linked with RelB activation, i.e. the actual RelB DNA binding activity. “RelB activation dependent gene” refers to a gene from said signature.

In a more preferred embodiment, the activation of the RelB protein is detected by determining the expression level of at least one gene selected in the group consisting of the SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-B gene, HLA-G gene, ABCG1 gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-A gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, IL27RA gene, DERL1 gene, YPEL3 gene, SENP7 gene, NSD3 gene, PCYT1A gene, TTC14 gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, USF3 gene, RDH10 gene, POP4 gene, MVB12A gene, LOC100506990 gene, MYLIP gene, KLKB1 gene, ZNF302 gene, ZNF302 gene, RAP1A gene, CD59 gene, SNX20 gene, SEMA4D gene, ZNF224 gene, RNASEL gene, ARSD gene, SLFN5 gene, ABCG1 gene, TTC39B gene, ABCG1 gene, ZNF81 gene, ATP5S gene, ZNF818P gene, ZNF829 gene, RGS3 gene, CECR7 gene, ZNF667-AS1 gene, ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SINHCAF gene, SLC25A33 gene, MFSD14C gene, C12orf73 gene, DNLZ gene, PTEN gene, DENR gene, MTFMT gene, CSKMT gene and ARHGAP30 gene.

In a another preferred embodiment, the activation of the RelB protein is detected by determining the expression level of at least one gene selected in the group consisting of the SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, RAP1A gene, SNX20 gene, SEMA4D gene, ZNF224 gene, RNASEL gene, ARSD gene, TTC39B gene, ZNF81 gene, ATP5S gene, ZNF818P gene, ZNF829 gene, RGS3 gene, CECR7 gene, ZNF667-AS1 gene, ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SLC25A33 gene, MFSD14C gene, C12orf73 gene, DNLZ gene, PTEN gene, DENR gene, MTFMT gene, CSKMT gene, ARHGAP30 gene, gene comprising in its Coding DNA Sequence (CDS) SEQ ID NO:3, gene comprising in its CDS, gene comprising in its CDS SEQ ID NO:47, gene comprising in its CDS SEQ ID NO:129, gene comprising in its CDS SEQ ID NO:135, gene comprising in its CDS SEQ ID NO:136 and gene comprising in its CDS SEQ ID NO:139.

In a preferred embodiment, the activation of the RelB protein is detected by determining the expression level of at least ten genes listed in the Table 2, or at least twenty genes listed in the Table 2, or at least thirty genes listed in the Table 2, or at least forty genes listed in the Table 2, or at least fifty genes listed in the Table 2, or at least sixty genes listed in the Table 2, or at least seventy genes listed in the Table 2, or at least eighty genes listed in the Table 2, or at least ninety genes listed in the Table 2, or at least one hundred genes listed in the Table 2, or at least one hundred and ten genes listed in the Table 2, or at least one hundred and twenty genes listed in the Table 2, or at least one hundred and thirty genes listed in the Table 2, more preferably one hundred and forty genes listed in the Table 2.Thus, the invention relates to a method for predicting the prognosis of a patient suffering from a B-cell lymphoma, comprising determining the expression level of at least one of said genes listed in Table 2 and/or any combination thereof, more preferably combination of all the genes, listed in table 2, or of all the genes from the activation RelB signature. In a preferred embodiment, the present invention also relates to a method for predicting the prognosis of a patient suffering from a B-cell lymphoma, consisting in determining the expression level of the genes as listed in Table 2 or described above (SEQ ID NO: 1-140).

In another embodiment of the invention, the activation of the RelB protein is detected by determining over-expression of at least one gene selected in the group consisting in the SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-B gene, HLA-G gene, ABCG1 gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-A gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, IL27RA gene, DERL1 gene, YPEL3 gene, SENP7 gene, NSD3 gene, PCYT1A gene, TTC14 gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, USF3 gene, RDH10 gene, POP4 gene, MVB12A gene, LOC100506990 gene, MYLIP gene, KLKB1 gene, ZNF302 gene, ZNF302 gene, RAP1A gene, CD59 gene, SNX20 gene, SEMA4D gene, ZNF224 gene, RNASEL gene, ARSD gene, SLFN5 gene, ABCG1 gene, TTC39B gene, ABCG1 gene, ZNF81 gene, ATP5S gene, ZNF818P gene, ZNF829 gene, RGS3 gene, CECR7 gene and ZNF667-AS1 gene, wherein over-expression of said genes is prognostic of a bad clinical outcome.

In another embodiment of the invention, the activation of the RelB protein is detected by determining over-expression of at least one gene selected in the group consisting of : SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, RAP1A gene, SNX20 gene, SEMA4D gene, ZNF224 gene, RNASEL gene, ARSD gene, TTC39B gene, ZNF81 gene, ATP5S gene, ZNF818P gene, ZNF829 gene, RGS3 gene, CECR7 gene, ZNF667-AS1 gene, gene comprising in its Coding DNA Sequence (CDS) SEQ ID NO:3, gene comprising in its CDS, gene comprising in its CDS SEQ ID NO:129, gene comprising in its CDS SEQ ID NO:136 and gene comprising in its CDS SEQ ID NO:139, wherein over-expression of said at least one gene is a prognosis of a bad clinical outcome.

In another embodiment of the invention, the activation of the RelB protein is detected by determining under-expression of at least one gene selected in the group consisting of: ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SINHCAF gene, SLC25A33 gene, MFSD14C gene, C12orf73 gene, DNLZ gene, PTEN gene, DENR gene, MTFMT gene, CSKMT gene and ARHGAP30 gene, wherein under-expression of said genes is prognostic of a bad clinical outcome.

In another embodiment of the invention, the activation of the RelB protein is detected by determining under-expression of at least one gene selected in the group consisting of : ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SLC25A33 gene, MFSD14C gene, C12orf73 gene, DNLZ gene, PTEN gene, DENR gene, MTFMT gene, CSKMT gene, ARHGAP30 gene, gene comprising in its CDS SEQ ID NO:47 and gene comprising in its CDS SEQ ID NO:135, wherein under-expression of at least one said gene is a prognosis of a bad clinical outcome.

In a preferred embodiment of the invention, the activation of the RelB protein is detected by determining over-expression of at least one gene selected in the group consisting in the SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-B gene, HLA-G gene, ABCG1 gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-A gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, IL27RA gene, DERL1 gene, YPEL3 gene, SENP7 gene, NSD3 gene, PCYT1A gene, TTC14 gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, USF3 gene, RDH10 gene, POP4 gene, MVB12A gene, LOC100506990 gene, MYLIP gene, KLKB1 gene, ZNF302 gene, ZNF302 gene, RAP1A gene, CD59 gene, SNX20 gene, SEMA4D gene, ZNF224 gene, RNASEL gene, ARSD gene, SLFN5 gene, ABCG1 gene, TTC39B gene, ABCG1 gene, ZNF81 gene, ATP5S gene, ZNF818P gene, ZNF829 gene, RGS3 gene, CECR7 gene and ZNF667-AS1 gene and determining under-expression of at least one gene selected in the group consisting in the ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SINHCAF gene, SLC25A33 gene, MFSD14C gene, C12orf73 gene, DNLZ gene, PTEN gene, DENR gene, MTFMT gene, CSKMT gene and ARHGAP30 gene, wherein said expression level of said genes is prognostic of a bad clinical outcome.

In another preferred embodiment of the invention, the activation of the RelB protein is detected by:
determining over-expression of at least one gene selected in the group consisting of : SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, RAP1A gene, SNX20 gene, SEMA4D gene, ZNF224 gene, RNASEL gene, ARSD gene, TTC39B gene, ZNF81 gene, ATP5S gene, ZNF818P gene, ZNF829 gene, RGS3 gene, CECR7 gene, ZNF667-AS1 gene, gene comprising in its Coding DNA Sequence (CDS) SEQ ID NO:3, gene comprising in its CDS, gene comprising in its CDS SEQ ID NO:129, gene comprising in its CDS SEQ ID NO:136 and gene comprising in its CDS SEQ ID NO:139, and
determining under-expression of at least one gene selected in the group consisting of : ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SLC25A33 gene, MFSD14C gene, C12orf73 gene, DNLZ gene, PTEN gene, DENR gene, MTFMT gene, CSKMT gene ARHGAP30 gene, gene comprising in its CDS SEQ ID NO:47 and gene comprising in its CDS SEQ ID NO:135,
   wherein said expression level of said genes is prognostic of a bad clinical outcome.

In another preferred embodiment of the invention, the activation of the RelB protein is detected by:
determining over-expression of SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, RAP1A gene, SNX20 gene, SEMA4D gene, ZNF224 gene, RNASEL gene, ARSD gene, TTC39B gene, ZNF81 gene, ATP5S gene, ZNF818P gene, ZNF829 gene, RGS3 gene, CECR7 gene, ZNF667-AS1 gene, gene comprising in its Coding DNA Sequence (CDS) SEQ ID NO:3, gene comprising in its CDS, gene comprising in its CDS SEQ ID NO:129, gene comprising in its CDS SEQ ID NO:136 and gene comprising in its CDS SEQ ID NO:139, and
determining under-expression of: ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SLC25A33 gene, MFSD14C gene, C12orf73 gene, DNLZ gene, PTEN gene, DENR gene, MTFMT gene, CSKMT gene ARHGAP30 gene, gene comprising in its CDS SEQ ID NO:47 and gene comprising in its CDS SEQ ID NO:135,
   wherein said expression level of said genes is prognostic of a bad clinical outcome.

In the method of the invention, if the said genes found over expressed in the RelB activation signature (see Table 2) are actually over-expressed in a subject as compared with a reference sample, then said subject is diagnosed as suffering from a B-cell lymphoma with a worse prognosis. In other words, an over-expression of SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAM P4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, RAP1A gene, SNX20 gene, SEMA4D gene, ZNF224 gene, RNASEL gene, ARSD gene, TTC39B gene, ZNF81 gene, ATP5S gene, ZNF818P gene, ZNF829 gene, RGS3 gene, CECR7 gene, ZNF667-AS1 gene, gene comprising in its Coding DNA Sequence (CDS) SEQ ID NO:3, gene comprising in its CDS, gene comprising in its CDS SEQ ID NO:129, gene comprising in its CDS SEQ ID NO:136 and gene comprising in its CDS SEQ ID NO:139 is regarded as an indicator of a worse prognosis for B-cell lymphoma. Alternatively, an over-expression of SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-B gene, HLA-G gene, ABCG1 gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-A gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, IL27RA gene, DERL1 gene, YPEL3 gene, SENP7 gene, NSD3 gene, PCYT1A gene, TTC14 gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, USF3 gene, RDH10 gene, POP4 gene, MVB12A gene, LOC100506990 gene, MYLIP gene, KLKB1 gene, ZNF302 gene, ZNF302 gene, RAP1A gene, CD59 gene, SNX20 gene, SEMA4D gene, ZNF224 gene, RNASEL gene, ARSD gene, SLFN5 gene, ABCG1 gene, TTC39B gene, ABCG1 gene, ZNF81 gene, ATP5S gene, ZNF818P gene, ZNF829 gene, RGS3 gene, CECR7 gene and ZNF667-AS1 gene is regarded as an indicator of a worse prognosis for B-cell lymphoma.

In another aspect, expression level of said genes over-expressed in RelB activation signature can be compared with the expression level of the same genes in a previously collected sample of said subject or in reference sample, and then said subject is diagnosed or not as suffering from a B-cell lymphoma with a worse prognosis.

In the method of the invention, if the genes found under-expressed in the RelB activation signature are found in a subject actually under expressed as compared with a reference sample, then said subject is diagnosed as suffering from a B-cell lymphoma with a worse prognosis. In other words, an under-expression of ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SLC25A33 gene, MFSD14C gene, C12orf73 gene, DNLZ gene, PTEN gene, DENR gene, MTFMT gene, CSKMT gene, ARHGAP30 gene, gene comprising in its CDS SEQ ID NO:47 and gene comprising in its CDS SEQ ID NO:135 as listed in Table 2, is regarded as an indicator of a worse prognostic for B-cell lymphoma. An under-expression of ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SinHCAF gene, SLC25A33 gene, MFSD14C gene, C12orf73 gene, DNLZ gene, PTEN gene, DENR gene, MTFMT gene, CSKMT gene, and ARHGAP30 gene is also regarded as an indicator of a worse prognosis for B-cell lymphoma.

In another aspect, expression level of said genes under-expressed in the RelB activation signature can be compared with the expression of the same genes of a previously collected sample of said subject or of a reference sample, and then said subject is diagnosed or not as suffering from a B-cell lymphoma with a worse prognosis.

According to any methods of the invention, if a gene is over-expressed and/or under-expressed as compared with a reference sample in accordance with the profile of RelB activation signature as mentioned in Table 2, then the subject is diagnosed as suffering from a B-cell lymphoma with a worse prognosis. In other words, an over-expression and an under-expression of genes of RelB activation signature as detailed in Table 2 below is regarded as an indicator of a worse prognosis for B-cell lymphoma. In another aspect, expression level of said genes of RelB activation signature can be compared with the same genes of a previously collected sample of said subject or of a reference sample, and then said subject is diagnosed or not as suffering from a B-cell lymphoma with a worse prognosis.

TABLE 2
List of genes of the RelB activation signature.

| Expression profile | Gene | Name | Sequences ID |
|---|---|---|---|
| OVER-EXPRESSION | SLAMF6 | SLAM family member 6 | SEQ ID NO: 1 |
| OVER-EXPRESSION | PSMB8-AS1 | PSMB8 antisense RNA 1 (head to head) | SEQ ID NO: 2 |
| OVER-EXPRESSION | — | — | SEQ ID NO: 3 |
| UNDER- | ZNF131 | zinc finger protein 131 | SEQ ID NO: 4 |
| EXPRESSION | | | |
| OVER-EXPRESSION | ZNF621 | zinc finger protein 621 | SEQ ID NO: 5 |
| UNDER- | LOC100506730 | uncharacterized LOC100506730 | SEQ ID NO: 6 |
| EXPRESSION | | | |
| UNDER- | TMEM67 | transmembrane protein 67 | SEQ ID NO: 7 |
| EXPRESSION | | | |
| UNDER- | PHF20 | PHD finger protein 20 | SEQ ID NO: 8 |
| EXPRESSION | | | |
| UNDER- | SLC25A3 | solute carrier family 25 member 3 | SEQ ID NO: 9 |
| EXPRESSION | | | |
| OVER-EXPRESSION | SARAF | store-operated calcium entry associated | SEQ ID NO: 10 |
| | | regulatory factor | |
| OVER-EXPRESSION | SSR1 | signal sequence receptor subunit 1 | SEQ ID NO: 11 |
| OVER-EXPRESSION | SLC9A3R1 | SLC9A3 regulator 1 | SEQ ID NO: 12 |
| OVER-EXPRESSION | SQSTM1 | sequestosome 1 | SEQ ID NO: 13 |
| OVER-EXPRESSION | RRAGA | Ras related GTP binding A | SEQ ID NO: 14 |
| OVER-EXPRESSION | CCND3 | cyclin D3 | SEQ ID NO: 15 |
| UNDER- | CCT2 | chaperonin containing TCP1 subunit 2 | SEQ ID NO: 16 |
| EXPRESSION | | | |
| OVER-EXPRESSION | PPP1R2 | protein phosphatase 1 regulatory | SEQ ID NO: 17 |
| | | inhibitor subunit 2 | |
| OVER-EXPRESSION | PPP1R2 | protein phosphatase 1 regulatory | SEQ ID NO: 18 |
| | | inhibitor subunit 2 | |
| OVER-EXPRESSION | HDAC5 | histone deacetylase 5 | SEQ ID NO: 19 |
| OVER-EXPRESSION | ITM2A | integral membrane protein 2A | SEQ ID NO: 20 |
| UNDER- | OXCT1 | 3-oxoacid CoA-transferase 1 | SEQ ID NO: 21 |
| EXPRESSION | | | |
| OVER-EXPRESSION | BIN1 | bridging integrator 1 | SEQ ID NO: 22 |
| UNDER- | LYPLA1 | lysophospholipase 1 | SEQ ID NO: 23 |
| EXPRESSION | | | |
| UNDER- | PAFAH1B3 | platelet activating factor acetylhydrolase | SEQ ID NO: 24 |
| EXPRESSION | | 1b catalytic subunit 3 | |
| UNDER- | PMVK | phosphomevalonate kinase | SEQ ID NO: 25 |
| EXPRESSION | | | |
| OVER-EXPRESSION | LPL | lipoprotein lipase | SEQ ID NO: 26 |
| UNDER- | PNO1 | partner of NOB1 homolog | SEQ ID NO: 27 |
| EXPRESSION | | | |
| OVER-EXPRESSION | FRY | FRY microtubule binding protein | SEQ ID NO: 28 |
| OVER-EXPRESSION | GMFG | glia maturation factor gamma | SEQ ID NO: 29 |
| OVER-EXPRESSION | TK2 | thymidine kinase 2, mitochondrial | SEQ ID NO: 30 |
| OVER-EXPRESSION | KIAA0513 | KIAA0513 | SEQ ID NO: 31 |
| OVER-EXPRESSION | ABCG1 | ATP binding cassette subfamily G | SEQ ID NO: 32 |
| | | member 1 | |
| OVER-EXPRESSION | HLA-F | major histocompatibility complex, class I, | SEQ ID NO: 33 |
| | | F | |
| OVER-EXPRESSION | BTN3A3 | butyrophilin subfamily 3 member A3 | SEQ ID NO: 34 |
| UNDER- | WRN | Werner syndrome RecQ like helicase | SEQ ID NO: 35 |
| EXPRESSION | | | |
| OVER-EXPRESSION | IL27RA | interleukin 27 receptor subunit alpha | SEQ ID NO: 36 |
| OVER-EXPRESSION | HCP5 | HLA complex P5 | SEQ ID NO: 37 |
| UNDER- | HOXC4 | homeobox C4 | SEQ ID NO: 38 |
| EXPRESSION | | | |
| UNDER- | CBFB | core-binding factor subunit beta | SEQ ID NO: 39 |
| EXPRESSION | | | |
| OVER-EXPRESSION | DZIP3 | DAZ interacting zinc finger protein 3 | SEQ ID NO: 40 |
| OVER-EXPRESSION | HLA-B | major histocompatibility complex, class I, | SEQ ID NO: 41 |
| | | B | |
| UNDER- | HNRNPR | heterogeneous nuclear | SEQ ID NO: 42 |
| EXPRESSION | | ribonucleoprotein R | |
| OVER-EXPRESSION | HLA-B | major histocompatibility complex, class I, | SEQ ID NO: 43 |
| | | B | |
| UNDER- | ATP2B1 | ATPase plasma membrane Ca2+ | SEQ ID NO: 44 |
| EXPRESSION | | transporting 1 | |
| OVER-EXPRESSION | HLA-G | major histocompatibility complex, class I, | SEQ ID NO: 45 |
| | | G | |
| OVER-EXPRESSION | ABCG1 | ATP binding cassette subfamily G | SEQ ID NO: 46 |
| | | member 1 | |
| UNDER- | — | — | SEQ ID NO: 47 |
| EXPRESSION | | | |
| OVER-EXPRESSION | HLA-G | major histocompatibility complex, , | SEQ ID NO: 48 |
| | | G | |
| UNDER- | UBE3A | ubiquitin protein ligase E3A | SEQ ID NO: 49 |
| EXPRESSION | | | |
| OVER-EXPRESSION | HLA-C | major histocompatibility complex, class I, | SEQ ID NO: 50 |
| | | C | |
| OVER-EXPRESSION | CD59 | CD59 molecule (CD59 blood group) | SEQ ID NO: 51 |
| OVER-EXPRESSION | STAT5B | signal transducer and activator of | SEQ ID NO: 52 |
| | | transcription 5B | |
| UNDER- | RCOR1 | REST corepressor 1 | SEQ ID NO: 53 |
| EXPRESSION | | | |
| OVER-EXPRESSION | BTN3A2 | butyrophilin subfamily 3 member A2 | SEQ ID NO: 54 |
| OVER-EXPRESSION | CSTF2T | cleavage stimulation factor subunit 2 tau | SEQ ID NO: 55 |
| | | variant | |
| UNDER- | SET | SET nuclear proto-oncogene | SEQ ID NO: 56 |
| EXPRESSION | | | |
| UNDER- | KNOP1 | lysine rich nucleolar protein 1 | SEQ ID NO: 57 |
| EXPRESSION | | | |
| OVER-EXPRESSION | CLIC5 | chloride intracellular channel 5 | SEQ ID NO: 58 |
| OVER-EXPRESSION | VAMP4 | vesicle associated membrane protein 4 | SEQ ID NO: 59 |
| OVER-EXPRESSION | HLA-A | major histocompatibility complex, class I, | SEQ ID NO: 60 |
| | | A | |
| OVER-EXPRESSION | N4BP2L2 | NEDD4 binding protein 2 like 2 | SEQ ID NO: 61 |
| OVER-EXPRESSION | HLA-A | major histocompatibility complex, class I, | SEQ ID NO: 62 |
| | | A | |
| OVER-EXPRESSION | HLA-J | major histocompatibility complex, class I, | SEQ ID NO: 63 |
| | | J (pseudogene) | |
| OVER-EXPRESSION | NDFIP1 | Nedd4 family interacting protein 1 | SEQ ID NO: 64 |
| UNDER- | MRPL42 | mitochondrial ribosomal protein L42 | SEQ ID NO: 65 |
| EXPRESSION | | | |
| UNDER- | RSF1 | remodeling and spacing factor 1 | SEQ ID NO: 66 |
| EXPRESSION | | | |
| OVER-EXPRESSION | ACSL5 | acyl-CoA synthetase long chain family | SEQ ID NO: 67 |
| | | member 5 | |
| OVER-EXPRESSION | FBXO3 | F-box protein 3 | SEQ ID NO: 68 |
| OVER-EXPRESSION | ZNF302 | zinc finger protein 302 | SEQ ID NO: 69 |
| OVER-EXPRESSION | ECHDC2 | enoyl-CoA hydratase domain containing | SEQ ID NO: 70 |
| | | 2 | |
| OVER-EXPRESSION | ARHGAP15 | Rho GTPase activating protein 15 | SEQ ID NO: 71 |
| OVER-EXPRESSION | NAP1L2 | nucleosome assembly protein 1 like 2 | SEQ ID NO: 72 |
| UNDER- | CCNJ | cyclin J | SEQ ID NO: 73 |
| EXPRESSION | | | |
| UNDER- | SINHCAF | SIN3-HDAC complex associated factor | SEQ ID NO: 74 |
| EXPRESSION | | | |
| UNDER- | PLEKHA8P1 | pleckstrin homology domain containing | SEQ ID NO: 75 |
| EXPRESSION | | A8 pseudogene 1 | |
| OVER-EXPRESSION | GVINPI | GTPase, very large interferon inducible | SEQ ID NO: 76 |
| | | pseudogene l | |
| OVER-EXPRESSION | FAM117A | family with sequence similarity 117 | SEQ ID NO: 77 |
| | | member A | |
| OVER-EXPRESSION | ZNF506 | zinc finger protein 506 | SEQ ID NO: 78 |
| UNDER- | BRIP1 | BRCA1 interacting protein C-terminal | SEQ ID NO: 79 |
| EXPRESSION | | helicase 1 | |
| OVER-EXPRESSION | IL27RA | interleukin 27 receptor subunit alpha | SEQ ID NO: 80 |
| OVER-EXPRESSION | DERL1 | derlin 1 | SEQ ID NO: 81 |
| UNDER- | SINHCAF | SIN3-HDAC complex associated factor | SEQ ID NO: 82 |
| EXPRESSION | | | |
| OVER-EXPRESSION | YPEL3 | yippee like 3 | SEQ ID NO: 83 |
| UNDER- | SLC25A33 | solute carrier family 25 member 33 | SEQ ID NO: 84 |
| EXPRESSION | | | |
| OVER-EXPRESSION | SENP7 | SUMO specific peptidase 7 | SEQ ID NO: 85 |
| OVER-EXPRESSION | NSD3 | nuclear receptor binding SET domain | SEQ ID NO: 86 |
| | | protein 3 | |
| UNDER- | MFSD14C | major facilitator superfamily domain | SEQ ID NO: 87 |
| EXPRESSION | | containing 14C | |
| OVER-EXPRESSION | PCYT1A | phosphate cytidylyltransferase 1, choline, | SEQ ID NO: 88 |
| | | alpha | |
| OVER-EXPRESSION | TTC14 | tetratricopeptide repeat domain 14 | SEQ ID NO: 89 |
| OVER-EXPRESSION | TTC14 | tetratricopeptide repeat domain 14 | SEQ ID NO: 90 |
| OVER-EXPRESSION | ARHGAP27 | Rho GTPase activating protein 27 | SEQ ID NO: 91 |
| OVER-EXPRESSION | MTMR10 | myotubularin related protein 10 | SEQ ID NO: 92 |
| OVER-EXPRESSION | FAM84B | family with sequence similarity 84 | SEQ ID NO: 93 |
| | | member B | |
| OVER-EXPRESSION | TRAPPC5 | trafficking protein particle complex 5 | SEQ ID NO: 94 |
| OVER-EXPRESSION | RDH10 | retinol dehydrogenase 10 | SEQ ID NO: 95 |
| OVER-EXPRESSION | FAM160B1 | family with sequence similarity 160 | SEQ ID NO: 96 |
| | | member B1 | |
| OVER-EXPRESSION | SLFN5 | schlafen family member 5 | SEQ ID NO: 97 |
| UNDER- | C12orf73 | chromosome 12 open reading frame 73 | SEQ ID NO: 98 |
| EXPRESSION | | | |
| OVER-EXPRESSION | NHLRC3 | NHL repeat containing 3 | SEQ ID NO: 99 |
| OVER-EXPRESSION | SUSD3 | sushi domain containing 3 | SEQ ID NO: 100 |
| OVER-EXPRESSION | FAM171B | family with sequence similarity 171 | SEQ ID NO: 101 |
| | | member B | |
| OVER-EXPRESSION | PLPP6 | phospholipid phosphatase 6 | SEQ ID NO: 102 |
| OVER-EXPRESSION | USF3 | upstream transcription factor family | SEQ ID NO: 103 |
| | | member 3 | |
| OVER-EXPRESSION | USF3 | upstream transcription factor family | SEQ ID NO: 104 |
| | | member 3 | |
| OVER-EXPRESSION | RDH10 | retinol dehydrogenase 10 | SEQ ID NO: 105 |
| OVER-EXPRESSION | POP4 | POP4 homolog, ribonuclease P/MRP | SEQ ID NO: 106 |
| | | subunit | |
| OVER-EXPRESSION | MVB12A | multivesicular body subunit 12A | SEQ ID NO: 107 |
| OVER-EXPRESSION | LOC100506990 | uncharacterized LOC100506990 | SEQ ID NO: 108 |
| OVER-EXPRESSION | MYLIP | myosin regulatory light chain interacting | SEQ ID NO: 109 |
| | | protein | |
| UNDER- | DNLZ | DNL-type zinc finger | SEQ ID NO: 110 |
| EXPRESSION | | | |
| OVER-EXPRESSION | KLKB1 | kallikrein Bl | SEQ ID NO: 111 |
| OVER-EXPRESSION | ZNF302 | zinc finger protein 302 | SEQ ID NO: 112 |
| OVER-EXPRESSION | ZNF302 | zinc finger protein 302 | SEQ ID NO: 113 |
| OVER-EXPRESSION | RAP1A | RAP1A, member of RAS oncogene family | SEQ ID NO: 114 |
| OVER-EXPRESSION | CD59 | CD59 molecule (CD59 blood group) | SEQ ID NO: 115 |
| OVER-EXPRESSION | SNX20 | sorting nexin 20 | SEQ ID NO: 116 |
| OVER-EXPRESSION | SEMA4D | semaphorin 4D | SEQ ID NO: 117 |
| OVER-EXPRESSION | ZNF224 | zinc finger protein 224 | SEQ ID NO: 118 |
| OVER-EXPRESSION | RNASEL | ribonuclease L | SEQ ID NO: 119 |
| OVER-EXPRESSION | ARSD | arylsulfatase D | SEQ ID NO: 120 |
| OVER-EXPRESSION | SLFN5 | schlafen family member 5 | SEQ ID NO: 121 |
| OVER-EXPRESSION | ABCG1 | ATP binding cassette subfamily G | SEQ ID NO: 122 |
| | | member 1 | |
| OVER-EXPRESSION | TTC39B | tetratricopeptide repeat domain 39B | SEQ ID NO: 123 |
| OVER-EXPRESSION | ABCG1 | ATP binding cassette subfamily G | SEQ ID NO: 124 |
| | | member 1 | |
| UNDER- | PTEN | phosphatase and tensin homolog | SEQ ID NO: 125 |
| EXPRESSION | | | |
| OVER-EXPRESSION | ZNF81 | zinc finger protein 81 | SEQ ID NO: 126 |
| UNDER- | DENR | density regulated re-initiation and | SEQ ID NO: 127 |
| EXPRESSION | | release factor | |
| UNDER- | MTFMT | mitochondrial methionyl-tRNA | SEQ ID NO: 128 |
| EXPRESSION | | formyltransferase | |
| OVER-EXPRESSION | — | — | SEQ ID NO: 129 |
| OVER-EXPRESSION | ATP5S | ATP synthase, H+ transporting, | SEQ ID NO: 130 |
| | | mitochondrial Fo complex subunit s | |
| | | (factor B) | |
| OVER-EXPRESSION | ZNF818P | zinc finger protein 818, pseudogene | SEQ ID NO: 131 |
| OVER-EXPRESSION | ZNF829 | zinc finger protein 829 | SEQ ID NO: 132 |
| UNDER- | CSKMT | citrate synthase lysine methyltransferase | SEQ ID NO: 133 |
| EXPRESSION | | | |
| OVER-EXPRESSION | RGS3 | regulator of G protein signaling 3 | SEQ ID NO: 134 |
| UNDER- | — | — | SEQ ID NO: 135 |
| EXPRESSION | | | |
| OVER-EXPRESSION | — | — | SEQ ID NO: 136 |
| OVER-EXPRESSION | CECR7 | cat eye syndrome chromosome region, | SEQ ID NO: 137 |
| | | candidate 7 | |
| UNDER- | ARHGAP30 | Rho GTPase activating protein 30 | SEQ ID NO: 138 |
| EXPRESSION | | | |
| OVER-EXPRESSION | — | — | SEQ ID NO: 139 |
| OVER-EXPRESSION | ZNF667-AS1 | ZNF667 antisense RNA 1 (head to head) | SEQ ID NO: 140 |

“n” in sequence listing represent regions that are not probed by the probe sequences.

In other words, wherein over or under expression of the RelB-dependent genes in said subject is determined by comparison with same genes in a reference sample in accordance with the RelB activation profile of Table 2, then said subject is diagnosed as suffering from a B-cell lymphoma with a worse prognosis.

A number of techniques have been proposed to detect expression of genes such as, for example RNAseq, DNA microarray, Nanostring or RT-MLPA to detect transcription of the gene.

DNA Microarray: A DNA microarray (also commonly known as DNA chip or biochip) is a collection of microscopic DNA spots attached to a solid surface. Scientists use DNA microarrays to measure the expression levels of large numbers of genes simultaneously or to genotype multiple regions of a genome.

RNAseq: RNA-Seq is a developed approach to transcriptome profiling that uses deep-sequencing technologies. It provides a precise measurement of levels of transcripts and their isoforms. (Wang Z et al., 2009).

Nanostring: NanoString's nCounter technology is a variation on the DNA microarray and was invented and patented by Krassen Dimitrov and Dwayne Dunaway. It uses molecular “barcodes” and microscopic imaging to detect and count up to several hundred unique transcripts in one hybridization reaction. (Geis, G K et al., 2008).

RT-MLPA: Multiplex ligation-dependent probe amplification (MLPA) is a variation of the multiplex polymerase chain reaction that permits amplification of multiple targets with only a single primer pair. (Schouten J P et al., 2002).

All these methods (RNAseq, microarray, Nanostring or RT-MLPA) can be used for determining the gene expression of the markers of the invention.

Other techniques can be used to detect gene expression of the signature of the invention, at the translational level, when gene is known to encode a protein.

These methods are well known from the skilled in art, they are for example protein microarrays such as RPPA or ELISA.

RPPA (Reverse Phase Protein Assay) is particularly suited for complex samples. After appropriate preparation, sample or lysate or extract thereof is arrayed onto the microarray and probed with antibodies against the target proteins of interest. These antibodies are typically detected with chemiluminescent, fluorescent or colorimetric assays. Reference peptides are printed on the slides to allow for protein quantification of the sample, lysate or extract. Thereby, RPPA allows the quantitative measurement of hundreds of proteins in biological and clinical samples (Boellner & Becker, 2015).

ELISA (enzyme-linked immunosorbent assay) is well known and can be used also to quantitate proteins. This assay uses a solid-phase type of enzyme immunoassay (EIA) to detect the presence of a protein in a liquid sample (lysate of cellular sample, fractionated liquid sample etc. . . . ) using antibodies directed against the protein to be measured.

The method of the invention requires to detect the “gene expression” or “level of expression” or “expression level”. According to the present invention, the terms “gene expression” or “level expression” refer to the phenotypic manifestation of a gene or genes by the processes of genetic transcription and genetic translation. When genes are expressed, the genetic information (base sequence) on DNA is first copied to a molecule of mRNA (transcription). The mRNA molecules then leave the cell nucleus and enter the cytoplasm, where they participate in protein synthesis by specifying the particular amino acids that make up individual proteins (translation). “gene expression” can be quantified according to technologies described above. In an embodiment expression of some of the genes of the signature of the invention can be detected/determined at the transcriptional level (e.g., quantifying mRNA or cDNA) and some other at the translational level (i.e. at the protein level). In a particular embodiment, expression of the gene of the signature of the invention is performed at the transcriptional level.

According to the present invention, the “reference sample” which is used to detect an “gene expression” or “level expression” for carrying out a diagnostic of prognosis of B-cell lymphoma or for following the evolution of prognosis of B-cell lymphoma is a biological sample from a subject that does not suffer from B-cell lymphoma or a biological sample from a subject who has been previously diagnosed as suffering from B-cell lymphoma but, e.g., whom history shown he was in complete remission or with a good clinical outcome. Accordingly, such as a normal or healthy cell or tissue or body fluid, or a data set produced using information from a normal or healthy cell or tissue or body fluid or a biological sample from a subject with a good clinical outcome.

In a preferred embodiment, the reference sample is a blood sample obtained from a healthy subject or from a subject with a good clinical outcome.

In a preferred embodiment, the level expression of the genes of RelB activation signature is determined by Nanostring or by RT-LMPA by using the primers or approaches targeting the 140 sequences of genes of SEQ ID NO:1-140.

In another preferred embodiment, the level expression of each of the genes of RelB activation signature is determined by Nanostring or by RT-MLPA by using the primers or approaches targeting one sequence per gene of the RelB activation signature amongst SEQ ID NO:1-140.

In another aspect, the signature of the invention can be used to predict the outcome of B-cell lymphoma cancer patients. Also, the signature of the invention can be used to aid the skilled oncologist in the selection of appropriate treatments for maximizing the survival of the patients. Appropriate treatments are for example chemotherapeutic treatments, immunotherapeutic treatments, radiotherapeutic treatments and/or surgery. Specifically, said patients have been treated or will be treated with chemotherapeutic drugs. As used herein, “treatments” may include some combination of surgery, chemotherapy, radiation therapy and targeted therapy. In another embodiment “treatments” may include to not chose, to change or to discontinue a treatment because of the diagnosis of worse prognosis of the tested subject.

The present invention also relates to a method for predicting a clinical outcome of a subject afflicted with B-cell lymphoma, said method comprising:
a. determining the level of expression of said genes of RelB signature in a biological sample of a said subject and comparing same to a reference value,
b. predicting the clinical outcome based on the comparison of step a).

If the following genes: SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-B gene, HLA-G gene, ABCG1 gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-A gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, IL27RA gene, DERL1 gene, YPEL3 gene, SENP7 gene, NSD3 gene, PCYT1A gene, TTC14 gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, USF3 gene, RDH10 gene, POP4 gene, MVB12A gene, LOC100506990 gene, MYLIP gene, KLKB1 gene, ZNF302 gene, ZNF302 gene, RAP1A gene, CD59 gene, SNX20 gene, SEMA4D gene, ZNF224 gene, RNASEL gene, ARSD gene, SLFN5 gene, ABCG1 gene, TTC39B gene, ABCG1 gene, ZNF81 gene, ATP5S gene, ZNF818P gene, ZNF829 gene, RGS3 gene, CECR7 gene and ZNF667-AS1 gene are significantly over-expressed and the 38 genes, ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SINHCAF gene, SLC25A33 gene, MFSD14C gene, C12orf73 gene, DNLZ gene, PTEN gene, DENR gene, MTFMT gene, CSKMT gene and ARHGAP30 gene are significantly under-expressed in the biological sample of the tested subject as compared to the same signature in a reference sample, then the tested subject is likely to have a bad clinical outcome.

If SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, RAP1A gene, SNX20 gene, SEMA4D gene, ZNF224 gene, RNASEL gene, ARSD gene, TTC39B gene, ZNF81 gene, ATP5S gene, ZNF818P gene, ZNF829 gene, RGS3 gene, CECR7 gene, ZNF667-AS1 gene, gene comprising in its Coding DNA Sequence (CDS) SEQ ID NO:3, gene comprising in its CDS, gene comprising in its CDS SEQ ID NO:129, gene comprising in its CDS SEQ ID NO:136 and gene comprising in its CDS SEQ ID NO:139 are significantly over-expressed and ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SLC25A33 gene, MFSD14C gene, C12orf73 gene, DNLZ gene, PTEN gene, DENR gene, MTFMT gene, CSKMT gene, ARHGAP30 gene, gene comprising in its CDS SEQ ID NO:47 and gene comprising in its CDS SEQ ID NO:135 are significantly under-expressed in the biological sample of the tested subject as compared to the same signature in a reference sample, then the tested subject is likely to have a bad clinical outcome.

Conversely, if gene expression profile of said genes described above are not significantly expressed in the biological sample of the tested subject as compared to the same signature in the reference sample, in accordance with expression profile of the RelB signature of the invention, then the tested subject is likely to have a good clinical outcome

In another aspect, the signature of the invention can be used for monitoring the evolution of B-cell lymphoma in a subject being diagnosed for B-cell lymphoma, said method comprising:
a) determining the status of DNA-binding activation of the RelB protein, in a biological sample of said subject, at a first time point,
b) determining the status of DNA-binding activation of the RelB protein, in a biological sample of said subject, at a second time point, and
c) comparing the status of DNA-binding activation of the RelB protein determined in step b) to the status of DNA-binding activation of the RelB protein determined in step a).

In a preferred embodiment, the biological sample in step a) is obtained prior to the treatment for B-cell lymphoma and the sample in step b) is obtained after said subject has been treated for B-cell lymphoma.

It can be concluded that the malignancy of the B-cell lymphoma is worsening if there is a DNA-binding activation of the RelB protein. In a preferred embodiment, the status of DNA-binding activation of the RelB protein is determined through the determination of the level of expression of the genes of the RelB signature according to the invention.

It can be concluded that the malignancy of the B-cell lymphoma is worsening if the expression level of the genes shown to be overexpressed in the RelB signature of Table 2 determined in step b) is found significantly higher than the expression level of same genes as determined in step a) and the expression level of the genes shown to be under expressed in the RelB signature of Table 2 determined in step b) is found significantly lower than the expression level of same gene in step a). In other words, the tested subject has a disease that evolves badly, even though he/she may be treated already, by conventional treatment (R-CHOP, R-ACVBP, . . . ), or more innovative immunotherapy such as, for example, CAR-T-cells treatment.

In another aspect, the signature of the invention can be used for determining or adapting a therapeutic regimen suitable for a subject diagnosed for B-cell lymphoma comprising the step of:
a. determining the status of DNA-binding activation of the RelB protein, in a biological sample of a subject prior to administration of treatment or during treatment of said subject,
b. determining the status of DNA-binding activation of the RelB protein, in a biological sample of the subject after administration of treatment of said subject,
c. comparing the status of DNA-binding activation determined in step b) to the status of DNA-binding activation determined in step a),
d. adapting/modifying the therapeutic regimen for the subject based on the comparison of step c).

In particular, said surgery or therapeutic regimen is efficient or suitable if there is no DNA-binding activation of the RelB protein. Conversely, said therapeutic regimen should have to be changed if there is a DNA-binding activation of the RelB protein.

In a preferred embodiment, the status of DNA-binding activation of the RelB protein is determined by determining the level of expression of the genes of RelB signature according to the invention.

In particular, said surgery or therapeutic regimen is efficient or suitable if, in the subject, the expression level of the genes of the signature does not match with the expression level of same genes consisting the signature in the reference sample.

More particularly, said surgery or therapeutic regimen is efficient or suitable if, in the subject:
the expression level of the genes described as over expressed in the RelB signature of the invention turn out to be significantly inferior to the expression level of same genes consisting the signature in the reference sample, and
the expression level of the genes described as under expressed in the RelB signature of the invention turn out to be significantly superior.

In particular, said surgery or therapeutic regimen is efficient or suitable if, in the subject, the expression level of the genes of the signature matches with the expression level of same genes consisting the signature in the reference sample.

In other words, said therapeutic regimen should have to be changed if, in the subject:
the expression level of the genes described as over expressed in the RelB signature of the invention of the invention turn out to be significantly over-expressed, and
and the expression level of the genes described as under expressed in the RelB signature of the invention turn out to be under-expressed,
   despite the presence of a treatment.

This aspect of treatment strategy is a crucial goal in a context of personalized medicine in order to improve survival while maintaining the quality of life and avoiding needless toxic effects of an ineffective treatment.

### Kits of the Invention

The present invention furthermore provides prognostic tools for determining the gene expression of the signature of the invention in order to prognose the outcome of B-cell lymphoma.

The present invention also relates to the use a kit for predicting a prognosis of subject suffering from B-cell lymphoma, comprising primers and/or nucleic acid probes targeting specifically the gene sequence of SEQ ID NO:1-140.

The present invention also relates to the use a kit for predicting a prognosis of subject suffering from B-cell lymphoma, comprising primers targeting specifically one sequence per gene of the RelB activation signature amongst SEQ ID NO:1-140.

The present invention also relates to the use a kit for predicting a prognosis of subject suffering from B-cell lymphoma, comprising primers targeting ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SLC25A33 gene, MFSD14C gene, C12orf73 gene, DNLZ gene, PTEN gene, DENR gene, MTFMT gene, CSKMT gene, ARHGAP30 gene, gene comprising in its CDS SEQ ID NO:47, gene comprising in its CDS SEQ ID NO:135, SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, RAP1A gene, SNX20 gene, SEMA4D gene, ZNF224 gene, RNASEL gene, ARSD gene, TTC39B gene, ZNF81 gene, ATP5S gene, ZNF818P gene, ZNF829 gene, RGS3 gene, CECR7 gene, ZNF667-AS1 gene, gene comprising in its Coding DNA Sequence (CDS) SEQ ID NO:3, gene comprising in its CDS, gene comprising in its CDS SEQ ID NO:129, gene comprising in its CDS SEQ ID NO:136 and gene comprising in its CDS SEQ ID NO:139.

As used herein, the term “kit” refers to any system for delivering materials. In the context of the invention, it includes systems that allow the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. The present kit can also include one or more reagents, buffers, hybridization media, nucleic acids, primers, nucleotides, probes, molecular weight markers, enzymes, solid supports, databases, computer programs for calculating dispensation orders and/or disposable lab equipment, such as multi-well plates, in order to readily facilitate implementation of the present methods. Enzymes that can be included in the present kits include nucleotide polymerases and the like. Solid supports can include beads and the like whereas molecular weight markers can include conjugatable markers, for example biotin and streptavidin or the like.

Further aspects and advantages of the invention will be disclosed in the following examples, which should be considered illustrative.

### EXAMPLE

### Material and Methods

### Patient Selection and Biopsies

Patients were selected from the GHEDI (Deciphering the Genetic Heterogeneity of Diffuse large B-cell lymphoma in the rituximab era) study program of the LYSA group, previously published and described (Dubois et al. 2016). Patients were enrolled in previous trials, with available frozen tumor samples, centralized histopathological review, adequate DNA/RNA quality and complete clinical information (Jais et al., 2017). COO molecular classification was obtained with HGU133+2.0 Affymetrix GeneChip arrays (Affymetrix), grouping patients into ABC, GCB and “unclassified”. We had access to 70 frozen samples from de novo DLBCL patients, as well as complete clinical and transcriptomic data of 202 patients. Patients included in each part of the study are listed on Table 1.

For all the analyses patients who received R-CHOP 14, R-CHOP 21 and mini-R-CHOP were grouped as “R-CHOP”. Patients who received R-ACVBP+conso, ACVBP+ASCT and ACVBP were grouped as “R-ACVBP”.

### Human DLBCL Cell Lines

DLBCL cell lines were obtained from José Angel Climent. Cells were grown in RPMI-1640 medium (Gibco glutamax) supplemented with 10% heat-inactivated fetal bovine serum (HyClone), 2 mM L-glutamine, 100 U/mL penicillin, and 100 mg/mL streptomycin (Invitrogen).

### Antibodies

The antibodies were purchased from Santa Cruz (RelA, RelB, p105/p50, p100/p52, c-Rel, Bcl-2), Sigma (β-actin) and Cell Signaling (cleaved caspase 3 (Asp175), Bcl-xL and clAP2).

### Immunoblotting

Immunoblotting were performed as typically known in the art.

### Lentiviral Production and Transduction

Production of infectious recombinant lentiviruses was performed by transient transfection of 293T cells. For infections, cells were incubated overnight with recombinant lentiviruses. An equal amount of fresh culture medium was added 24 hours later and after 48 hours, cells were washed and seeded in fresh culture medium. GFP positive cells were sorted with FACSAria™ sorter (Becton Dickinson). Only cells with a high expression of GFP, above 40000 of mean fluorescence intensity (MFI), were selected and then amplified.

### Anexin V Binding Assay

Cells were harvested and washed twice with cold PBS. They were resuspended in 1× binding buffer containing Annexin V-APC (BD Biosciences Pharmingen) and 4′,6-diaminidino-2-phenylindole (DAPI, Molecular Probes) following the manufacturer's instructions. The samples were subjected to cytometric analysis with a MACSQuant cytometer (Miltenyi Biotec) and the data was statistically evaluated using the Flowjo v10.2 software.

### γH2AX Foci

Cells incubated with or without doxorubicin were fixated in formalin 4%, permeabilized in SDS 1%, and then stained with an anti-γH2AX antibody. Samples were incubated with secondary antibody Alexa Fluor 647® following analysis using an ImageStream X Mark II Imaging Flow Cytometer. Data were acquired at a 60× magnification with EDF using the 642 nm laser at 150 mW and INSPIRE software. At least 8000 events of cells per sample were analyzed. Acquired data were analyzed using the IDEAS analysis software (v6.1; Merck-Millipore). Cells were gated for focused cells using the Gradient RMS feature. Cells were gated for single cells using the aspect ratio and area features. The spot counting analysis wizard was used.

### Electrophoretic Mobility Shift Assays for NF-κB

For electrophoretic mobility shift assay (EMSA) we used the human immunodeficiency virus long terminal repeat tandem κB oligonucleotide as κB probe. For supershift assays, total protein extracts were incubated with specific antibodies (Santa Cruz—RelA C-20X; RelB C-19X; c-Rel (C)X; p50 H-119X; p52 K-27X). Cases were analyzed and classified by 3 different researchers independently. A consensus was achieved for the discordant cases.

### RT-q PCR

Total RNA extraction and reverse transcription were performed. Real-time PCR analysis was carried out with LightCycler FastStart DNA Master plus SYBR Green I on a Light Cycler 1.5 (Roche Applied Science). All values were normalized to the level of HPRT mRNA.

### p53 Functional Status

p53 gene functional status is determined by the functional analysis of separated alleles in yeast (FASAY) method. p53 status was considered mutated when: (a) >10% of the yeast colonies are red, (b) analysis using the split versions of the test could identify the defect in the 5′ or 3′ part of the gene, and (c) sequence analysis from mutant yeast colonies (Sanger) could identify an unambiguous genetic defect.

### Immunohistochemistry

Immunoperoxidase staining was centrally performed on an Ultra auto- mated system (Roche Ventana, Tucson, Ariz.) using UltraVIEW detection Original kits and optimized protocols for BCL-2, and MYC staining. In the absence of an internal positive control, immunostains were considered non-evaluable. The tissue core with the highest percentage of tumor cell staining was considered for analysis. The thresholds employed were 40% for MYC and 50% or 70% for BCL2.

### Statistical Analysis

### Cell Death

Statistical significance was assessed using unpaired t tests (Prism 5.0c, GraphPad Software). A value of p=0.05 was considered as statistically significant with the following degrees: *p<0.05; **p<0.01; ***p<0.001.

### Clinical Analysis

Statistical analyses were performed using the software Statistical Package for the Social Sciences (SPSS, version 24), including descriptive statistics and statistical tests (chi-square for categorical variables or Fisher's exact test when Chi-square was not appropriate; T test for comparison of means; Kaplan-Meier method for evaluating prognostic impact of predictors; Cox proportional hazard model for survival-time outcomes on one or more predictors). Survival curves were created for overall survival based on Kaplan-Meier method, according to clinical data obtained from GHEDI cohort, previously described elsewhere (39)Cox proportional hazard regression analyses for univariate and multivariate were based on forward stepwise regression, with a cutoff for p-values≤0.2. Two-side P-values<0.05 were considered statistically significant (p=0.05).

### Transcriptomic Analysis

Microarray experiments were performed on Affymetrix Human Genome HGU133plus2.0 GeneChips (a genome wide array with 54674 probe sets targeting 19418 transcripts). Gene expression levels were normalized using the GC-RMA algorithm and flags were computed using MAS5. Quality assessment of the chips has been performed with affyQCReport R package. MAS5 algorithm produces a flag “P” for “Present”, “M” for “Marginal” or “A” for “Absent” associated to each intensity measure. This flag is an estimation of the statistical difference between PM (Perfect Match) and MM (Mismatch). Three probe lists have been used for each comparison according to flagged measurement in the relevant chips. The “PP” list is made of probes only flagged as “Present” for all chips involved in the comparison. The “P50” list has been created filtering probes flagged as “Present” for at least half of the chips. The “All” list is made of all probes without any filter. Three groups of two biologically independent samples were compared. The group comparisons were done using Student's t test. To estimate the false discovery rate we filtered the resulting p values at 5% and used the Benjamini and Hochberg (BH), Bonferroni (B) or without correction (SC). Cluster analysis was performed by hierarchical clustering using the Spearman correlation similarity measure and average linkage algorithm. Data were subsequently submitted to Ingenuity Pathway Analysis (IPA) to model relationships among genes and proteins and to construct putative pathways and relevant biological processes.

### Results

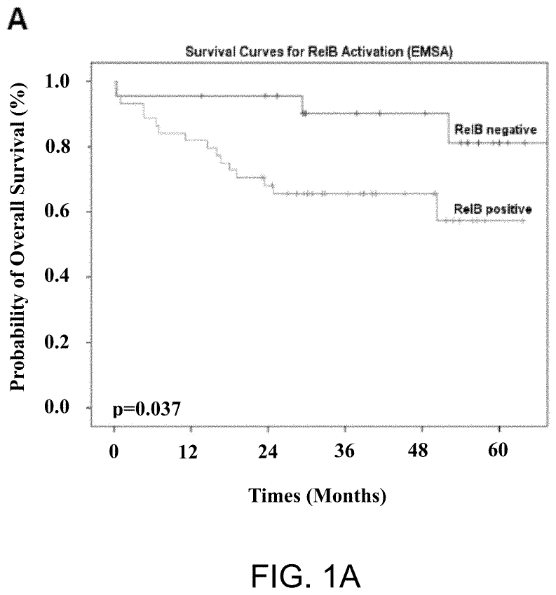

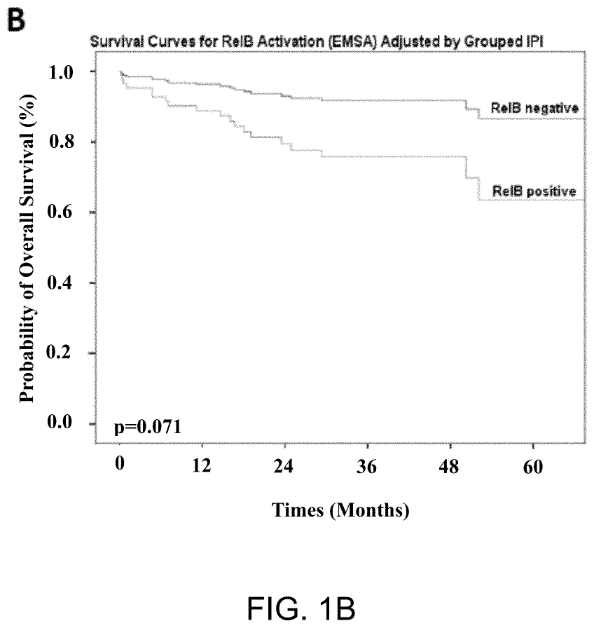

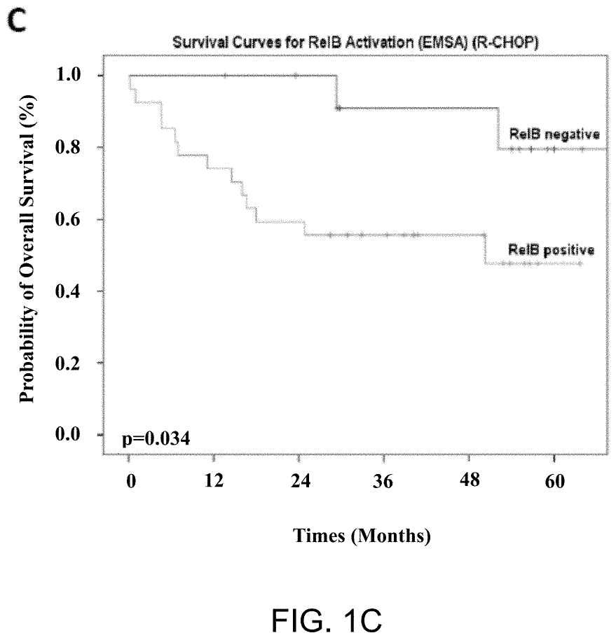

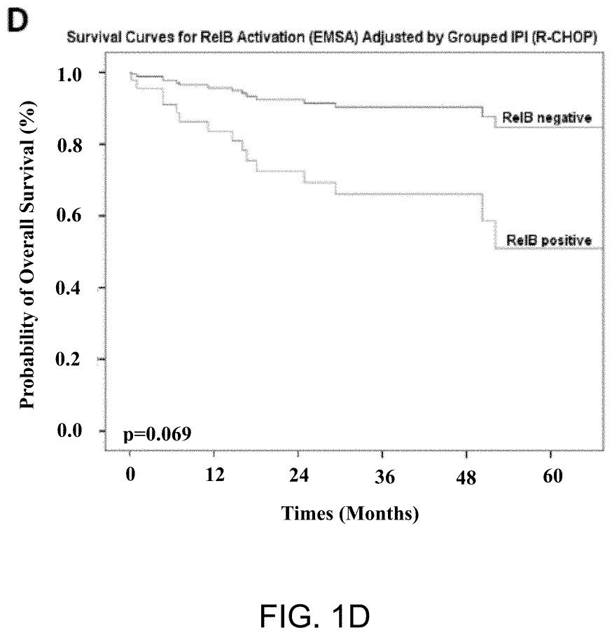

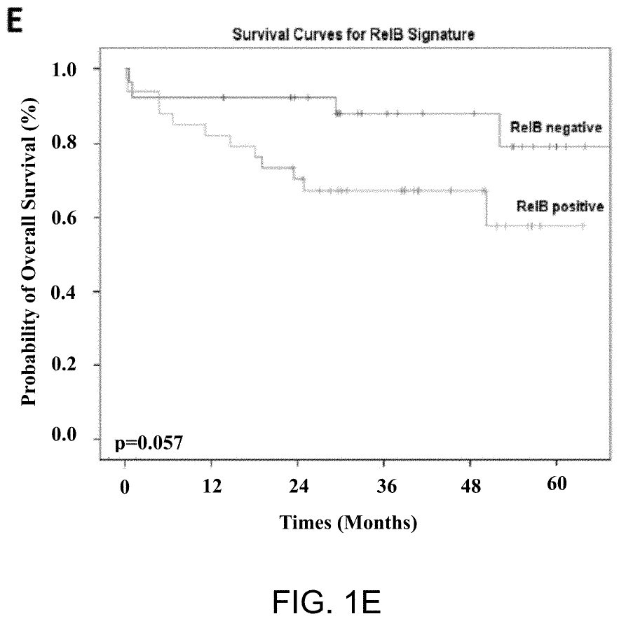

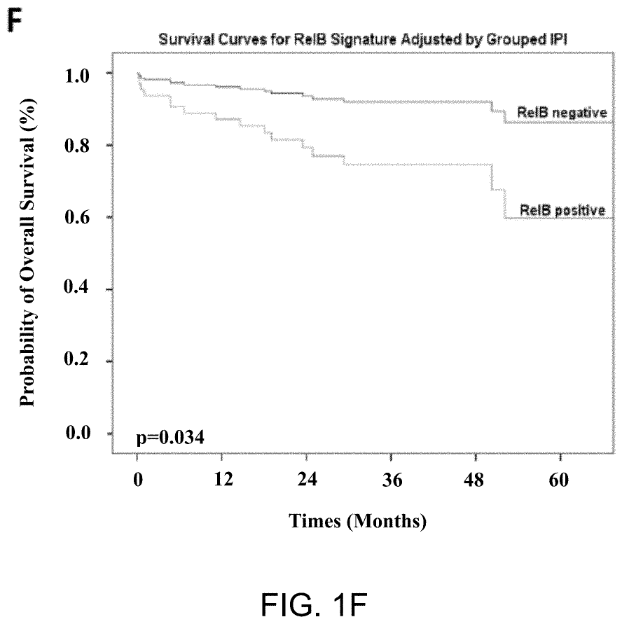

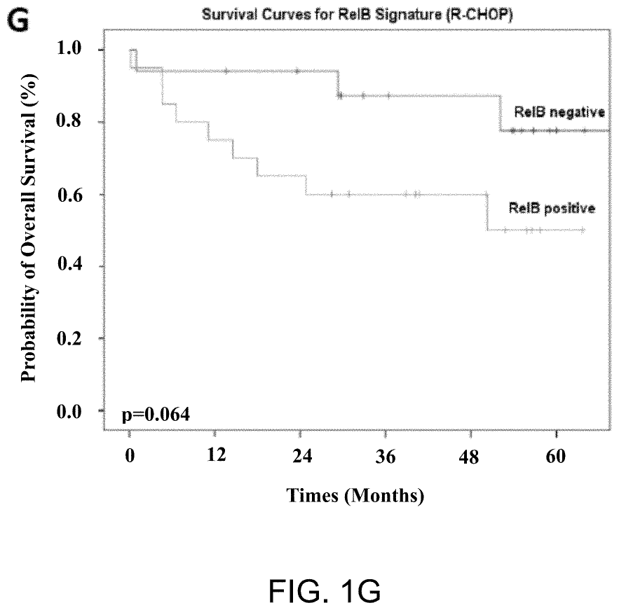

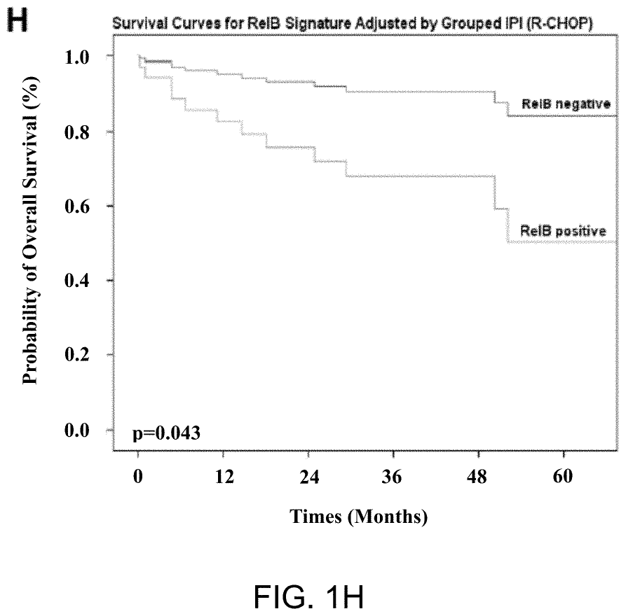

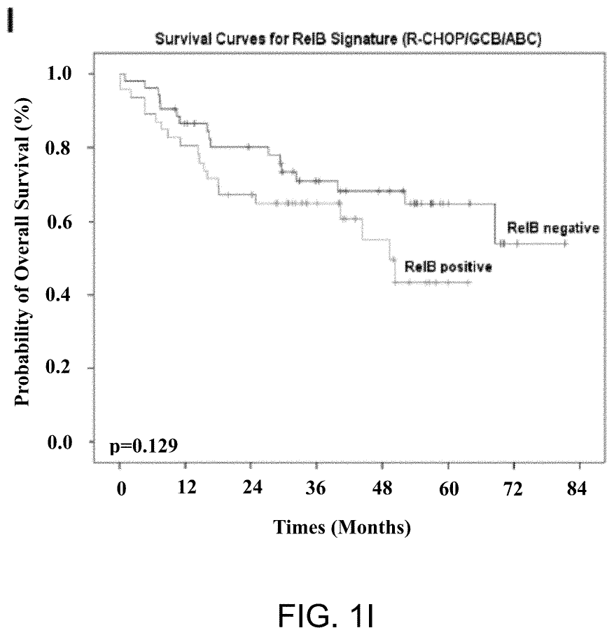

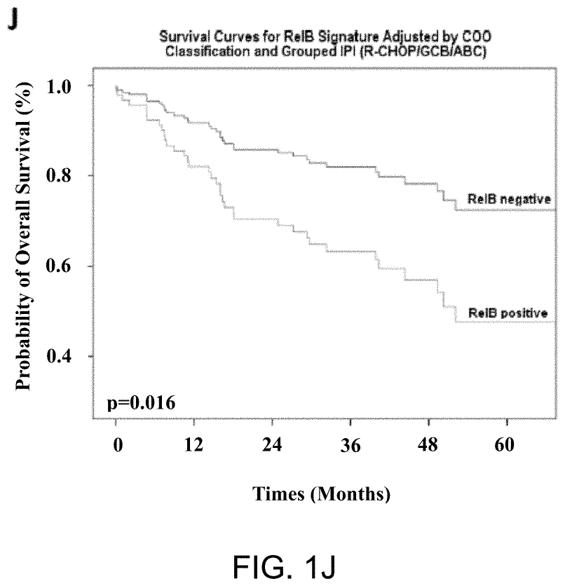

RelB activation correlated with worse overall survival (OS) (p=0.037) in the 66 patients analyzed. Multivariate Cox regression analysis showed similar tendency as in Kaplan-Meier (KM) survival curves when adjusted by grouped IPI (p=0.071) (FIG. 1A and 1B). Prognostic predictors were included in multivariate analysis when a 0.2 significance level was reached by univariate Cox regression (RelB EMSA: p=0.049; grouped IPI: p=0.002; COO classification: p=0.505) (Table 3). When selected only patients treated with R-CHOP regimen, the trend is still maintained in OS p=0.034 and when adjusted by grouped IPI (p=0.069) (FIG. 1C and 1D).

TABLE 3
Univariate analysis of studied cohorts.

| | | | Multivariate Cox HR Regression | | | | |
|---|---|---|---|---|---|---|---|
| Tested Cohort | | | β | HR | 95% CI LL | 95% CI UL | p-value |
| Test | Total | RelB EMSA (negative) | 1 | | | | |
| Cohort | (n = 66) | RelB EMSA (positive) | −1.154 | 0.315 | 0.09 | 1.105 | 0.071 |
| EMSA | | Grouped IPI (low risk) | 1 | | | | |
| | | Grouped IPI (high risk) | −2.25 | 0.105 | 0.024 | 0.459 | 0.003 |
| Test | R-CHOP | RelB EMSA (negative) | 1 | | | | |
| Cohort | (n = 40) | RelB EMSA (positive) | −1.403 | 0.246 | 0.054 | 1.117 | 0.069 |
| EMSA | | Grouped IPI (low risk) | 1 | | | | |
| | | Grouped IPI (high risk) | −2.248 | 0.106 | 0.014 | 0.814 | 0.031 |
| Test | Total for | RelB GEP (negative) | 1 | | | | |
| Cohort | signature | RelB GEP (positive) | −1.25 | 0.286 | 0.09 | 0.911 | 0.034 |
| GEP | (n = 61) | Grouped IPI (low risk) | 1 | | | | |
| | | Grouped IPI (high risk) | −2.196 | 0.111 | 0.025 | 0.494 | 0.004 |
| Test | R-CHOP | RelB GEP (negative) | 1 | | | | |
| Cohort | (n = 37) | RelB GEP (positive) | −1.385 | 0.25 | 0.066 | 0.956 | 0.043 |
| GEP | | Grouped IPI (low risk) | 1 | | | | |
| | | Grouped IPI (high risk) | −2.314 | 0.099 | 0.012 | 0.784 | 0.029 |
| Extended | R-CHOP | RelB GEP (negative) | 1 | | | | |
| Validation | GCB/ABC | RelB GEP (positive) | −0.832 | 0.435 | 0.221 | 0.858 | 0.016 |
| Cohort | (n = 98) | Grouped IPI (low risk) | 1 | | | | |
| | | Grouped IPI (high risk) | −0.787 | 0.455 | 0.218 | 0.951 | 0.003 |
| | | COO (GCB) | 1 | | | | |
| | | COO (ABC) | −1.812 | 0.163 | 0.049 | 0.541 | 0.036 |

Further, we established a gene expression profile (GEP) of genes associated with RelB activation in the EMSA-tested cohort. RelB-specific GEP was then projected on EMSA tested cases. The RelB-specific GEP was able to reproduce the EMSA established RelB activation status with 85% of sensitivity, 100% of specificity and a positive predictive value of 100%. RelB positive group defined by GEP revealed a tendency to a worse outcome in Kaplan-Meier survival curves (p=0.057). When adjusted by grouped IPI, RelB determined a worse outcome (p=0.034) (FIG. 1E and 1F). In a similar way to cases defined by EMSA, when taken only the group of R-CHOP treated patients, the trend was also maintained. RelB determined a worse OS (p=0.064) and was a stronger marker when adjusted by grouped IPI (p=0.043) (FIG. 1G and 1H).

In a second step, RelB transcriptomic signature was extended to a larger cohort (98), including the patients from the training cohort. We have excluded patients not treated by R-CHOP, since it is the main first line therapy for DLBCL, in which the main drug is doxorubicin further tested in DLBCL cell lines in our study. We also excluded patients unclassifiable as either GCB or ABC, because the signature was established in a training cohort containing only one of unclassified case. We observed a tendency of worse outcome in RelB positive group (p=0.129). When adjusted also by COO classification and grouped IPI in multivariate Cox regression analysis, RelB positivity defined a group of poorer prognosis (p=0.016) (FIGS. 1I and 1J).

Next, we have evaluated in our training cohort of 66 DLBCL patients whether RelB activation is linked to the published NF-κB transcriptional signature composed of six genes (Davis et al., 2001). Remarkably, RelB activation status was not linked to the expression of any of these genes, indicating that the actual NF-κB signature does not reflect the status of RelB activation. As described, this so-called NF-κB signature marked preferentially the ABC DLBLC with an overexpression of four genes out of six (p<0.05). When comparing patients with strong RelA vs cRel activity, four genes showed significant overexpression in the RelA group (p<0.05) which is in line with RelA activation correlating with the ABC subtype (Table 4). Importantly, RelB activation did not correlate with any of the mutations commonly associated with NF-κB in DLBCL (e.g. MYD88, CARD11, CD79A/B, TNFAIP3). Altogether, these observations indicate that the current tools to evaluate NF-κB activity only apply to the classical NF-κB pathway and do not provide valuable information on RelB activation status.

TABLE 4
NF-kB DLBCL gene expression signature from Davis et al.
(2001) comparison by groups.

| | Gene expression comparison | | |
|---|---|---|---|
| | ABC vs GCB | sRelA vs scRel | RelB pos vs RelB neg |
| Bcl-2 | p < 0.001 | p = 0.76 | p = 0.27 |
| Cyclin D2 | p < 0.001 | p < 0.001 | p = 0.93 |
| CCR7 | p = 0.63 | p = 0.21 | p = 0.85 |
| c-FLIP | p = 0.027 | p = 0.01 | p = 0.50 |
| IRF4 | p = 0.001 | p = 0.002 | p = 0.91 |
| IκBα | p = 0.76 | p = 0.03 | p = 0.43 |

Expression of Bcl-2 and c-Myc was assessed by IHC in 47 and 45 cases, respectively. There was no association between Bcl-2, c-Myc or double expression with RelB activation status (p=1.0, p=0.375, p=0.096 respectively). Double expression of c-Myc and Bcl-2 by IHC did not affect the overall survival of patients (p=0.652).

Altogether, our study shed light for the first time on the frequent engagement of RelB DNA binding activity in a cohort of DLBCL patients which is associated with a worse prognosis independently from COO classification and grouped IPI. Further, we identified a RelB transcriptomic signature associated with a prognosis value on a larger validation cohort.

### CONCLUSION

Inventor has demonstrated for the first time by direct DNA binding that RelB is frequently activated in DLBCL patients. RelB activation defined by EMSA was associated with poorer outcome. The direct assessment of RelB activation status allowed the definition of a RelB signature that was able to predict RelB activation status and define a group of worse prognosis in a larger validation cohort. RelB prognostic value was statistically significant when adjusted by grouped IPI and COO classification. Finally, RelB is a new prognostic marker for DLBCL patients and might open the road for improvement in patient stratification and new targeted therapy.

### REFERENCES

Alizadeh A A, Eisen M B, Davis R E, et al. Distinct types of diffuse large B-cell lymphoma identified by gene expression profiling. Nature. 2000;403(6769):503-511.

Boellner S, Becker K F. Reverse Phase Protein Arrays-Quantitative Assessment of Multiple Biomarkers in Biopsies for Clinical Use. Microarrays (Basel). 2015;4(2):98-114.

Davis R E, Brown K D, Siebenlist U, Staudt L M. Constitutive Nuclear Factor κB Activity Is Required for Survival of Activated B Cell—like Diffuse Large B Cell Lymphoma Cells. J Exp Med [Internet]. 2001;194(12):1861-74. De Leval L, Harris N L. Diffuse large B cell lymphomas. Lymphoid Neoplasms 3ed. 2010;50 :560-86.

Dubois S, Viailly P-J Mareschal S, Bohers E, Bertrand P, Ruminy P, et al. Next generation sequencing in diffuse large B cell lymphoma highlights molecular divergence and therapeutic opportunities: a LYSA study. Clin Cancer Res 2016;22:2919-28.

Geiss G K, Bumgarner R E, Birditt B, et al. Direct multiplexed measurement of gene expression with color-coded probe pairs. Nat Biotechnol. 2008;26(3):317-325.

Jacque E, Tchenio T, Piton G, Romeo P H, Baud V. RelA repression of RelB activity induces selective gene activation downstream of TNF receptors. Proc Natl Acad Sci USA. 2005;102(41):14635-14640.

Jais J-P, Molina T J, Ruminy P, Gentien D, Reyes C, Scott D W, et al. Reliable subtype classification of diffuse large B-cell lymphoma samples from GELA LNH2003 trials using the Lymph2Cx gene expression assay. Haematologica [Internet]. 2017 October;102(10):e404-6.

Schouten J P, McElgunn C J, Waaijer R, Zwijnenburg D, Diepvens F, Pals G. Relative quantification of 40 nucleic acid sequences by multiplex ligation-dependent probe amplification. Nucleic Acids Res. 2002;30(12):e57.

Edited by Swerdlow S H, Campo E, Harris N L, Jaffe E S, Pileri S A, Stein H, Thiele J. WHO Classification of Tumours of Haematopoietic and Lymphoid Tissues. 2017 WHO Classification of Tumours, Revised 4th Edition, Volume 2.

Wang Z, Gerstein M, Snyder M. RNA-Seq: a revolutionary tool for transcriptomics. Nat Rev Genet. 2009;10(1):57-63.

Reference to a “Sequence Listing,” a Table, or a Computer Program Listing Appendix submitted as an ASCII Text File

The material in the ASCII text file, name “APIC-65499-Sequence-Listing_ST25.txt”, created December 17, 2021, file size 122,880 bytes, is hereby incorporated by reference.

1. An in vitro method for predicting the prognosis of a subject suffering from a B-cell lymphoma, said method comprising the step of detecting the status of DNA-binding activation of the RelB protein in a biological sample of said subject.

2. The method according to claim 1, wherein the activation of the RelB protein is detected by measuring the expression level of at least one RelB activation-dependent gene from an expression signature in the biological sample of said subject.

3. The method according to claim 2, comprising determining the expression level of at least one gene selected from the group consisting of the SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene, ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, ZNF302 gene, RAP1A gene, SNX20 gene, SEMA4D gene, ZNF224 gene, RNASEL gene, ARSD geneTTC39B gene, ZNF81 gene, ATP5S gene, ZNF818P gene, ZNF829 gene, RGS3 gene, CECR7 gene, ZNF667-AS1 gene, ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, PLEKHA8P1 gene, BRIP1 gene, SINHCAF gene, SLC25A33 gene, MFSD14C gene, C12orf73 gene, DNLZ gene, PTEN gene, DENR gene, MTFMT gene, CSKMT geneARHGAP30 gene, gene comprising in its Coding DNA Sequence (CDS) SEQ ID NO:3, gene comprising in its CDS SEQ ID NO:47, gene comprising in its CDS SEQ ID NO:129, gene comprising in its CDS SEQ ID NO:135, gene comprising in its CDS SEQ ID NO:136, and gene comprising in its CDS SEQ ID NO:139.

4. The method according to claim 2, comprising determining over-expression of at least one gene selected from the group consisting of the SLAMF6 gene, PSMB8-AS1 gene, ZNF621 gene, SARAF gene, SSR1 gene, SLC9A3R1 gene, SQSTM1 gene, RRAGA gene, CCND3 gene, PPP1R2 gene, PPP1R2 gene, HDAC5 gene, ITM2A gene, BIN1 gene, LPL gene, FRY gene, GMFG gene, TK2 gene, KIAA0513 gene, ABCG1 gene, HLA-F gene, BTN3A3 gene, IL27RA gene, HCP5 gene, DZIP3 gene, HLA-B gene, HLA-G gene, HLA-C gene, CD59 gene, STAT5B gene, BTN3A2 gene, CSTF2T gene, CLIC5 gene, VAMP4 gene, HLA-A gene, N4BP2L2 gene, HLA-J gene, NDFIP1 gene, ACSL5 gene, FBXO3 gene, ZNF302 gene, ECHDC2 gene, ARHGAP15 gene, NAP1L2 gene, GVINP1 gene, FAM117A gene, ZNF506 gene, DERL1 gene, YPEL3 gene, SENP7 gene, NSD3 gene, PCYT1A gene, TTC14 gene,ARHGAP27 gene, MTMR10 gene, FAM84B gene, TRAPPC5 gene, RDH10 gene, FAM160B1 gene, SLFN5 gene, NHLRC3 gene, SUSD3 gene, FAM171B gene, PLPP6 gene, USF3 gene, POP4 gene, MVB12A gene, LOC100506990 gene, MYLIP gene, KLKB1 gene, ZNF302 gene, RAP1A gene, SNX20 gene, SEMA4D gene, ZNF224 gene, RNASEL gene, ARSD gene, TTC39B gene, ABCG1 gene, ZNF81 gene, ATPSS gene, ZNF818P gene, ZNF829 gene, RGS3 gene, CECR7 gene, ZNF667-AS1 gene, gene comprising in its Coding DNA Sequence (CDS) SEQ ID NO:3, gene comprising in its CDS SEQ ID NO:129, gene comprising in its CDS SEQ ID NO:136 and gene comprising in its CDS SEQ ID NO:139, wherein over-expression of said at least one gene is prognostic of a bad clinical outcome.

5. The method according to claim 2, comprising determining under-expression of at least one gene selected from the group consisting of the ZNF131 gene, LOC100506730 gene, TMEM67 gene, PHF20 gene, SLC25A3 gene, CCT2 gene, OXCT1 gene, LYPLA1 gene, PAFAH1B3 gene, PMVK gene, PNO1 gene, WRN gene, HOXC4 gene, CBFB gene, HNRNPR gene, ATP2B1 gene, UBE3A gene, RCOR1 gene, SET gene, KNOP1 gene, MRPL42 gene, RSF1 gene, CCNJ gene, SINHCAF gene, PLEKHA8P1 gene, BRIP1 gene, SLC25A33 gene, MFSD14C gene, C12orf73 gene, DNLZ gene, PTEN gene, DENR gene, MTFMT gene, CSKMT gene ARHGAP30 gene, gene comprising in its CDS SEQ ID NO:47 and gene comprising in its CDS SEQ ID NO:135, wherein under-expression of said at least one gene is prognostic of a bad clinical outcome.

6. The method according to claim 2, wherein over or under expression of the at least one RelB activation-dependent gene in said subject is determined by comparison with the same at least one RelB activation-dependent gene in a reference sample, and then said subject is diagnosed as suffering from a B-cell lymphoma with a worse prognosis based on the over or under expression.

7. The method according to claim 2, wherein said biological sample is a body effluent or tumor sample of said subject.

8. The method according to claim 7, wherein said body effluent is urine or blood sample.

9. The method according to claim 8, wherein said tumor sample is biopsy or surgical/resected specimen.

10. The method according to claim 2, wherein the at least one RelB activation-dependent gene expression signature is determined by RNAseq, microarray, Nanostring or RT-LMPA.

11. The method according to claim 1, wherein the subject is suffering from Diffuse Large B cell Lymphoma (DLBCL).

12. An in vitro method for monitoring the evolution of B-cell lymphoma in a subject being diagnosed for B-cell lymphoma, said method comprising:
a) determining the status of DNA-binding activation of the RelB protein, in a biological sample of said subject, at a first time point,
b) determining the status of DNA-binding activation of the RelB protein, in a biological sample of said subject, at a second time point, and
c) comparing the status of DNA-binding activation of the RelB protein determined in step b) to the status of DNA-binding activation of the RelB protein determined in step a).

13. The method according to claim 12, wherein the biological sample in step a) is obtained prior to the treatment for B-cell lymphoma and the sample in step b) is obtained after said subject has been treated for B-cell lymphoma.

14. A method for determining or adapting a therapeutic regimen suitable for a subject diagnosed for B-cell lymphoma comprising the steps of:
a. determining the status of DNA-binding activation of the RelB protein, in a biological sample of a subject prior to administration of treatment or during treatment of said subject,
b. determining the status of DNA-binding activation of the RelB protein, in a biological sample of the subject after administration of treatment of said subject,
c. comparing the status of DNA-binding activation determined in step b) to the status of DNA-binding activation determined in step a), and
d. adapting/modifying the therapeutic regimen for the subject based on the comparison of step c).

15. (canceled)


SEQUENCE LISTING (140 sequences)

SEQ ID NO: 1 - DNA, 471 bp, Artificial Sequence
  Synthetic construct SLAMF6
tagcattacccttctgacactctctatgtagcctccctgatcttctttcagctcctctattaaaggaaaagttctttatgttaattatttacatcttcctgcaggcccttcctctgcctgctggggtcctcctattctttaggtttaattttaaatatgtcacctcctaagagaaaccttcccagaccactctttctaaaatgaatcttctaggctgggcatggtggctcacacctgtaatcccagtactttgggaggccaaggggggagatcacttgaggtcaggagttcaagaccagcctggccaacttggtgaaaccccgtctttactaaaaatacaaaaaaattagccaggcgtggtggtgcacccctaaaatcccagctacttgagagactgaggcaggagaatcgcttgaacccaggaggtggaggttccagtgagccaaaatcatgccaatgtattccagtctg

SEQ ID NO: 2 - DNA, 354 bp, Artificial Sequence
  Synthetic construct PSMB8-AS1
  misc_feature (152)..(152): n is a or t or c or g
  misc_feature (157)..(157): n is a or t or c or g
  misc_feature (169)..(169): n is a or t or c or g
ccattctgagtacttctccgcaaaccctttgtttcattaaggactgttttacatgaagggtgcaaaagtaggataaaaatgagaaccctagggtgaaacacgtgacagaagaataaagactattgaatagtcctcttctctacccatggacnttggnatttttatattngattttaaggaaatataacttagtagtaaagagatgagcattcaagtcaggcagacctgaatttgggtcaaggctgcgccactcaaaagctatatgacctctatatgagcagcttattcaacctcttttaacctccattttgtcatctgtagaatgatgataaatgcctagctcagaaggattcc

SEQ ID NO: 3 - DNA, 551 bp, Artificial Sequence
  Synthetic construct nd
  misc_feature (94)..(94): n is a or t or c or g
  misc_feature (137)..(137): n is a or t or c or g
ccagcctctctgtacaaattttttgccacacaaatctgaaaataccaccttgatttatggatcctccaagtttttcactgaagtacaagggaanaaatgtaaatctataaattgggttaaatgatttctaggaaagngtataataataataacaattatttttttgagacagagtctctgtcacccaggctggagtgaaactgtgtctcaaaataaataaataaataataaataaataaataatagaaatgaagttttgctatagttgagaaattatagaatgccatgcttattatgataacctcatacagaaatttttacagctttttgaaaaatggcttttttgttattcaatttttatagtcttataaacattaattttcacaagactagtgtaaaaatattgtagacaatttgtctaagcagtatttcggaagcactttcaacagttatgtaaagggtcctatttatgcttgaagaaaggacatgtataaggtttttagcatgaggaaattaagtctttctcaacctagaagtcaggagtttcaaga

SEQ ID NO: 4 - DNA, 314 bp, Artificial Sequence
  Synthetic construct ZNF131
  misc_feature (280)..(280): n is a or t or c or g
aagtgagaatctaactgcccagatgatagtggtttctactacaaagcaagcctccatgaattcattcacacctgaggtttattaaatatgtgaccccaggcgggttactttacctcaattgcatacttttctcctcatctgaaaagtgggttcataggattataaagactaattgcgataacatgcagagtgcatggcatgatatttggtgccttgtcaaagcttgataaacagggtccagtctaagaaacattagggagacctcttctctacaaaaaantttaaaaaattattagctgagtctgatggcacat

SEQ ID NO: 5 - DNA, 469 bp, Artificial Sequence
  Synthetic construct ZNF621
gtggaacctgcatatgtgactgaattgctgcaatctcatgataaaacgtgaacaaatgaggagttgcttctaatataagagccacaaaagtggtttcttgagatggaaactactcctggtgaagatcctgtgaccattgttgaaatgacaacaaaggacttagaatattacataaacttagttgataaaacagcagcagagtttgagaggattgacttcaattttgaagaatgttccactgtgggtaaaaggctatccaacagcaacacatgctacagataaatctttcggaaaggaagagtcagttgatgttgcagccttccttgttgtctcatttaaagaaatctctagagccacactaaccttcagcaaccaccacaccgatcattcagcagccatcaacagtgaggcaagactgtcttccagcaacaaagactcactgaagacttagattatcgttagcatcttt

SEQ ID NO: 6 - DNA, 286 bp, Artificial Sequence
  Synthetic construct LOC100506730
  misc_feature (232)..(232): n is a or t or c or g
tgagctactgctcctggcctatggatgccctttatcagtttcaggaattcccttctattcctaattggctgagaggtttattttcctttcatgaataggtattaaattttgtcaaatgctttttctgcatctattgagatggtgatatgggttttgtcttatagcctgttagtatagtaaattatattgattgattttaaaatattaagccaaccttgtattcctgggatgnaactaacttggtcatggtgcattattgtttgtatttactactgtattcattttg

SEQ ID NO: 7 - DNA, 345 bp, Artificial Sequence
  Synthetic construct TMEM67
  misc_feature (145)..(145): n is a or t or c or g
  misc_feature (168)..(168): n is a or t or c or g
  misc_feature (178)..(178): n is a or t or c or g
  misc_feature (184)..(184): n is a or t or c or g
  misc_feature (226)..(226): n is a or t or c or g
  misc_feature (231)..(231): n is a or t or c or g
  misc_feature (254)..(254): n is a or t or c or g
  misc_feature (260)..(260): n is a or t or c or g
aaaactcttaatattgtaggattagatggttatttccttaacataacatgacaaaatatgtctcaactcaaaagccaacttgatacttaatagggaaaaagcgtaagcattcctgttaatgtcaggacctggaaaaggatgcaanctatgcacttaagtaagagaaanaaaatatttntcattnggaaaagaaaaggtaggccgttgttaatcctagcactttggnaaggntaaggcaggtgtattgcttgagnctcaangagtttgagaccagtctgggcaacatggtgaaaccgcatctctacaaataaaaaaaaaaaattatttggttacggtggcatgcac

SEQ ID NO: 8 - DNA, 93 bp, Artificial Sequence
  Synthetic construct PHF20
ggagaataaaggcagccccgttgatgactgaaaatgacaaagcatccacctaacagacgaggaatcagctttgaagtgggagcccagttggaa

SEQ ID NO: 9 - DNA, 342 bp, Artificial Sequence
  Synthetic construct SLC25A3
acaccatgatgaagttcgcctgctttgaacgtactgttgaagcactgtacaagtttgtggttcctaagccccgcagtgaatgttcaaagccagagcagctggttgtaacatttgtagcaggttacatagctggagtcttttgtgcaattgtttctcaccctgctgattctgtggtatctgtgttgaataaagaaaaaggtagcagtgcttctctggtcctcaagagacttggatttaaaggtgtatggaagggactgtttgcccgtatcatcatgattggtaccctgactgcactacagtggtttatctatgactccgtgaaggtctacttcagacttcctc

SEQ ID NO: 10 - DNA, 450 bp, Artificial Sequence
  Synthetic construct SARAF
atttctaaaacctgtgatgccctaagaagcattaagaatgaaggtgttgtactaatagaaactaagtacagaaatttcagttttaggtggttgtagctgatgagttattacctcatagagactataatattctatttggtattatattatttgatgtttgctgttcttcaaacatttaaatcaagctttggactaattatgctaatttgtgagttctgatcacttttgagctctgaagctttgaatcattcagtggtggagatggccttctggtaactgaatattaccttctgtaggaaaaggtggaaaataagcatctagaaggttgttgtgaatgactctgtgctggcaaaaatgcttgaaacctctatatttctttcgttcataagaggtaaaggtcaaatttttcaacaaaagtcttttaataacaaaagcatgcagttctctgtg

SEQ ID NO: 11 - DNA, 233 bp, Artificial Sequence
  Synthetic construct SSR1
aattcaattccacacatggtttgttcaagcacacttaataagtagcctattttttaaatgtctttttaaaatgtaaatatttggatgaagtttttctttgttttgatatattcatttgctacaccaactatgttttcagaattcatcttttgaacaacttggtttcagaatatgtaaaatgactttaaggatcttgtgtatcaaacctatccccggatgtgtgagaataatgt

SEQ ID NO: 12 - DNA, 526 bp, Artificial Sequence
  Synthetic construct SLC9A3R1
agagaactatgttcttccctgactttagggaaggtgaatgtgttcccgtcctcccgcagtcagaaaggagactctgcctccctcctcctcactgagtgcctcatcctaccgggtgtccctttgccaccctgcctgggacatcgctggaacctgcaccatgccaggatcatgggaccaggcgagagggcaccctcccttcctcccccatgtgataaatgggtccagggctgatcaaagaactctgactgcagaactgccgctctcagtggacagggcatctgttatcctgaaccttggcagacacgtcttgttttcatttgattttgttaagagtgcagtattgcagagtctagaggaatttttgtttccttgattaacatgattttcctggttgttaatccagggcatggcagtggcctcagccttaaacttttgttcctactcccaccctcagcgaactgggcagcacggggagggtttggctacccctgcccatccctgagccaggtaccaccattgtaaggaa

SEQ ID NO: 13 - DNA, 483 bp, Artificial Sequence
  Synthetic construct SQSTM1
ctgccttcttccaggatcaggggttagggtgcaagaagccatttagggcagcaaaacaagtgacatgaagggagggtccctgtgtgtgtgtgtgctgatgtttcctgggtgccctggctccttgcagcagggctgggcctgcgagacccaaggctcactgcagcgcgctcctgacccctccctgcaggggctacgttagcagcccagcacatagcttgcctaatggctttcactttctcttttgttttaaatgactcataggtccctgacatttagttgattattttctgctacagacctggtacactctgattttagataaagtaagcctaggtgttgtcagcaggcaggctggggaggccagtgttgtgggcttcctgctgggactgagaaggctcacgaagggcatccgcaatgttggtttcactgagagctgcctcctggtctcttcaccactgtagttctctcatttccaaaccatca

SEQ ID NO: 14 - DNA, 533 bp, Artificial Sequence
  Synthetic construct RRAGA
gagctgcagtaaattggccgcttccttccagagcatggaagttaggaattccaacttcgctgctttcatcgacatcttcacctcaaatacgtacgtgatggtggtcatgtcagatccgtcgatcccttctgcggccactctgatcaacattcgcaatgcccggaaacactttgagaagctggagagagtggatggccccaagcacagtctccttatgcgttgaatattgccaaatgctctttctgaaaatgctgaattgccttttttgtttgcatcctttatttttaatattcataatgtcgtgtgcttaaaagtgggctttgaagtgtgtgctgcttactcctttcatctttctccccgcttccccagtctttaaacattggacgctatttactcagctacccagtagagcttgaagctgacctttctgagaagttggtatggtgtaacactaaagtaggtggttcgtgtgtgttctcattacctggttatgatagatatgcacatcaaagcctttaccagtatcttcctgt

SEQ ID NO: 15 - DNA, 546 bp, Artificial Sequence
  Synthetic construct CCND3
ttgcatttggattggggtccctctaaaatttaatgcatgatagacacatatgagggggaatagtctagatggctcctctcagtactttggaggcccctatgtagtccgtgctgacagctgctcctagagggaggggcctaggcctcagccagagaagctataaattcctctttgctttgctttctgctcagcttctcctgtgtgattgacagctttgctgctgaaggctcattttaatttattaattgctttgagcacaactttaagaggacataatgggggcctggccatccacaagtggtggtaaccctggtggttgctgttttcctcccttctgctactggcaaaaggatctttgtggccaaggagctgctatagcctggggtggggtcatgccctcctctcccattgtccctctgccccatcctccagcagggaaaatgcagcagggatgccctggaggtggctgagcccctgtctagagagggaggcaagccctgttgacacaggtctttcctaaggctgcaaggtttaggctggtggccc

SEQ ID NO: 16 - DNA, 572 bp, Artificial Sequence
  Synthetic construct CCT2
ggtgttgacaatccagcagctaaagttttagttgatatgtcaagggttcaagatgatgaagttggtgatggcactacctctgttaccgttttagcagcagaattattaagggaagcagaatctttaattgcaaaaaagattcatccacagaccatcatagcgggttggagagaagccacgaaggctgcaagagaggcgctgttgagttctgcagttgatcatggttccgatgaagttaaattccgtcaagatttaatgaatattgcgggcacaacattatcctcaaaacttcttactcatcacaaagaccactttacaaagttagctgtagaagcagttctcagactgaaaggctctggcaacctggaggcaattcatattatcaagaagctaggaggaagtttggcagattcctatttagatgaaggcttcctgttggataaaaaaattggagtaaatcaaccaaaacgaattgaaaatgctaaaattcttattgcaaatactggtatggatacagacaaaataaagatatttggttcccgggtaagagttgactctacagcaaaggttgc

SEQ ID NO: 17 - DNA, 468 bp, Artificial Sequence
  Synthetic construct PPP1R2
  misc_feature (83)..(83): n is a or t or c or g
  misc_feature (84)..(84): n is a or t or c or g
  misc_feature (85)..(85): n is a or t or c or g
  misc_feature (135)..(135): n is a or t or c or g
  misc_feature (139)..(139): n is a or t or c or g
  misc_feature (145)..(145): n is a or t or c or g
  misc_feature (205)..(205): n is a or t or c or g
gcacatatattcatgcaccctctgattttggttttcttgtttctgagttcttagaaagtacccacatactctttttttttttnnngtagaagtagctgtcatagagtgaagaaaaggataagactttaaacagtngatncttttngtgttttctacaaactattttttgaaatttaaatcacaaactcattttctggtttttagnaaagtagatgatgatttcagaggagtaaggcatgccaagcagcatgctcagtggggttttaggctgtcacatgcagctgagaaaaggtatgttcaagtcataagtaggtaattgatagggtatgaactagtcaaaatatgaaccattatgattcaagttagattttcctctggagagacagatctgaatgttcagttctagccaaggtagattttactttcaactttttaatcagtatcactttctgtgcttaactatttggt

SEQ ID NO: 18 - DNA, 424 bp, Artificial Sequence
  Synthetic construct PPP1R2
ggatctactccaagtgaccaacagcaaaacaaattacgaagttcatagacgagatttgttcaacactgcaattgtttgttagatgtaaaccctgtgactatagtacgttgcttcttgttcttcacaattcatgacttaagtaccaaaatgcataccagttattatatattgccaagaattaaatgataaacttagagactgattagactgaaaatgcctaatcgatatatatattcttgtgcctagtactttaccacaaatacagtgtaatatcatcagtccaaaactgcattacttttgtaaaaacactggttaatttgtataagatattatagagctttttatgctttagaagttaaacaatatctttgggggggaactaatttattttcatcacttgaaatgtggtagctcttacaaagtt

SEQ ID NO: 19 - DNA, 521 bp, Artificial Sequence
  Synthetic construct HDAC5
acgagttctcacctgatgtggtcctagtctccgccgggtttgatgctgttgaaggacatctgtctcctctgggtggctactctgtcaccgccagatgttttggccacttgaccaggcagctgatgaccctggcagggggccgggtggtgctggccctggagggaggccatgacttgaccgccatctgtgatgcctctgaggcttgtgtctcggctctgctcagtgtagagctgcagcccttggatgaggcagtcttgcagcaaaagcccaacatcaacgcagtggccacgctagagaaagtcatcgagatccagagcaaacactggagctgtgtgcagaagttcgccgctggtctgggccggtccctgcgagaggcccaagcaggtgagaccgaggaggccgagactgtgagcgccatggccttgctgtcggtgggggccgagcaggcccaggctgcggcagcccgggaacacagccccaggccggcagaggagcccatggagcaggagcctgccctgtga

SEQ ID NO: 20 - DNA, 549 bp, Artificial Sequence
  Synthetic construct ITM2A
  misc_feature (76)..(76): n is a or t or c or g
aaactactaaccactgcaagctcttgtcaaattttagtttaattggcattgcttgttttttgaaactgaaattacntgagtttcattttttctttgaatttatagggtttagatttctgaaagcagcatgaatatatcacctaacatcctgacaataaattccatccgttgttttttttgtttgtttgttttttcttttcctttaagtaagctctttattcatcttatggtgcagcaattttaaaatttgaaatattttaaattgtttttgaactttttgtgtaaaatatatcagatctcaacattgttggtttcttttgtttttcattttgtacaactttcttgaatttagaaattacatctttgcagttctgttaggtgctctgtaattaacctgacttatatgtgaacaattttcatgagacagtcatttttaactaatgcagtgattctttctcactactatctgtattgtggaatgcacaaaattgtgtaggtgctgaatgctgtaaggagtttaggttgtatgaattctacaaccctataata

SEQ ID NO: 21 - DNA, 460 bp, Artificial Sequence
  Synthetic construct OXCT1
ttctcaatacctgacaggggcccatgggaatgacttcagaagcatcccggataatagatgggtaaaaagtctaggcaccctgaagaacaggtgagacagctggcctctggacagaggtaggcatagtacagtacgatatatcattcctctggtcctaaatatacaaacttattcatgtttttaggtgatgatggtcattgaaactcacttcttttcaggtgtagctacaattgtgtaatgtacaatattagagaaaggacaggctttttatgagtaacacacaccatatataaaacagcctttctggctgaccacatggttaaatgcataccttcccagtactggggggaaaatgacccttcttagaatgtgcaagttccatagagtaatatattgatatgattttgaaaagaattgttgatagttacatcttcaaacttatcattccagtatgcatctt

SEQ ID NO: 22 - DNA, 470 bp, Artificial Sequence
  Synthetic construct BIN1
tcttccccgagaacttcactgagagggtcccatgacggcggggcccaggcagcctccgggcgtgtgaagaacacctcctcccgaaaaatgtgtggttcttttttttgttttgttttcgtttttcatcttttgaagagcaaagggaaatcaagaggagacccccaggcagaggggcgttctcccaaagtttaggtcgttttccaaagagccgcgtcccggcaagtccggcggaattcaccagtgttcctgaagctgctgtgtcctctagttgagtttctggcgcccctgcctgtgcccgcatgtgtgcctggccgcagggcggggctgggggctgccgagccaccatacttaactgaagcttcggccgcaccacccggggaagggtcctcttttcctggcagctgctgtgggtggggcccagacaccagcctagcctgctctgccccgcagacggtctgtgtgctgtttga

SEQ ID NO: 23 - DNA, 549 bp, Artificial Sequence
  Synthetic construct LYPLA1
ccatgcccagtcttcaaatttctaatgtttgcagtgtttaaatgttttgcaaatacatgccattaacacagatcaataatatctcctctgagaatttatgatcttaagtctatacatgtattcttataagacgacccaggatctactatattagaatagatgaagcaggtagcttcttttttctcaaatgtaattcagcaaaataatacagtactgccaccagattttttattacatcatttgaaaattagcagtatgcttaatgaaaatttgttcaggtataaatgagcagttaagatataaacaatttatgcatgctgtgacttagtctatggatttattccaaaattgcttagtcaccatgcagtgtctgtatttttatatatgtgttcatatatacataatgattataatacataataagaatgaggtggtattacattattcctaataatagggataatgctgtttattgtcaagaaaaagtaaaatcgttctcttcaattaatggcccttttattttgggaccaggcttttattttccctgat

SEQ ID NO: 24 - DNA, 509 bp, Artificial Sequence
  Synthetic construct PAFAH1B3
gcaccagtgcgagatctggcgcgagctcttctctcctctgcatgcacttaactttggcattggtggtgacggcacacagcatgtactgtggcggctggagaatggggagctggaacacatccggcccaagattgtggtggtctgggtgggcaccaacaaccacggacacacagcagagcaggtgactggtggcatcaaggccattgtgcaactggtgaatgagcgacagccccaggcccgggttgtggtgctgggcctgcttccgcgaggccaacatcccaacccacttcgggagaagaaccgacaggtgaacgagctggtacgggcggcactggctggccaccctcgggcccacttcctagatgccgaccctggctttgtgcactcagatggcaccatcagccatcatgacatgtatgattacctgcatctgagccgcctgggctacacacctgtttgccgggctctgcactccctgcttctgcgtctgctggcccaagaccagggcc

SEQ ID NO: 25 - DNA, 543 bp, Artificial Sequence
  Synthetic construct PMVK
acggtccgcgttgtagcgttggagcagagccgacagcagcggggctgggtgttcacgccaggggtggacgatgctgagtcagaatgtggcctggacaacttcggggactttgactgggtcatcgagaaccatggagttgaacagcgcctggaggagcagttggagaacctgatagaatttatccgctccagactttagtcactaggttctaggagtgagctggggcctgctgaggtgggggtggggctgactctgcaaaatgggggtgtcccccgatcctggccgaggtgaggaacagacagggggggtctagattctgagggggttggtggatattgggcaaggcaggaaacctctggagacctcattttctccatggggaagacagccatgctcttcaggaggagactccaagggcaaaggagggtgtcttggctgtgcttgaaggcgaaaccctgccatatccccagtgccagtcccctcagcctgtggtggccttgcatcctgactggatgttctcagccccttgttctgggcaagaac

SEQ ID NO: 26 - DNA, 436 bp, Artificial Sequence
  Synthetic construct LPL
tcctgatgtgccagaacttcgaccctttctctgagagagatgatcgtgcctataaatagtaggaccaatgttgtgattaacatcatcaggcttggaatgaattctctctaaaaataaaatgatgtatgatttgttgttggcatcccctttattaattcattaaatttctggatttgggttgtgacccagggtgcattaacttaaaagattcactaaagcagcacatagcactgggaactctggctccgaaaaactttgttatatatatcaaggatgttctggctttacattttatttattagctgtaaatacatgtgtggatgtgtaaatggagcttgtacatattggaaaggtcattgtggctatctgcatttataaatgtgtggtgctaactgtatgtgtctttatcagtgatggtctcacagagccaactcac

SEQ ID NO: 27 - DNA, 431 bp, Artificial Sequence
  Synthetic construct PNO1
tgtgaaagttcacatccttggctccttccaaaatatcaagatggcaagaactgccatttgcaacctaatcttgggaaatcctccttccaaggtttatggcaatattcgagctgtggctagcagatcagcagatcgattctgatttcaagtcagagactttttatcttgcctttggactctggtgaaaaatactttacagtggtcggtcacaagaaaccatctgaacaatttcagtcatttgaagcctccgtcccttcttccattctcagccagaagcataaacagaaaagaaagatttaagaggattcacactcaacaggttttaggataatttaaatatcaaaaattgattgttatacttacacattaggtataatttatcatttatctgaaatcacatgtagcagattgcatagtctgtaatcctctca

SEQ ID NO: 28 - DNA, 433 bp, Artificial Sequence
  Synthetic construct FRY
ggagctgctatgaagtacctttcttatgttgctaggctactgtttctgaaagccctggatctctttgcaccaaaaatggtccagatagactctttttaaggatcttggctgctttttactagaaggttgcttttatgagcatatttatactgctgaaggatgagtgttaattttaattaactttgccgttttgtagagaaaactattcacaagataaattccaagtcttttcacctgtcaggcatgcatattttaatatctgtttggatagtcagaagtagaatcataaaggtaaaatatgagttgttactttgtttcttcgatgtcatattttatgtgtaatatatatgtaaagggccattcttaagttctctccttaaacttaatgctgtcaagtgttagatgtgtgcatgtgaacttgttgcactgcaga

SEQ ID NO: 29 - DNA, 488 bp, Artificial Sequence
  Synthetic construct GMFG
ggtgtgcgaggtagacccagagctaacagaaaagctgaggaaattccgcttccgaaaagagacagacaatgcagccatcataatgaaggtggacaaagaccggcagatggtggtgctggaggaagaatttcagaacatttccccagaggagctcaaaatggagttgccggagagacagcccaggttcgtggtttacagctacaagtacgtgcatgacgatggccgagtgtcctaccctttgtgtttcatcttctccagccctgtgggctgcaagccggaacaacagatgatgtatgcagggagtaaaaacaggctggtgcagacagcagagctcacaaaggtgttcgaaatccgcaccactgatgacctcactgaggcctggctccaagaaaagttgtctttctttcgttgatctctgggctggggactgaattcctgatgtctgagtcctcaggtgactggggacttggaaccctaggacctgaaca

SEQ ID NO: 30 - DNA, 523 bp, Artificial Sequence
  Synthetic construct TK2
ttatgctgcatttggctggagcccggtgttcagtggtttccctgcccgaggtcgctgcagccccatctaccacatcttcatgtggacattgagattcacatgctggctcctgaagggtgctcagtctccttggtgattaaggtcctgcttgaactgctgccaactccatgtcagggaagtcgcttttggtgcctggctggtttgcccagagccaagctggggcaaggggcagccagccctggcttccaaggctcccgtactgtctgtgtccttgtataaggagctttgctcttggaattactgaaagtctgtggccctaagagagagacacaagtggccttaagtctttttgaagtgttatttcatccagggaaatgcctcgagccatagagcctgaaatcatctttgttggctcagaaaataccttagcttcactcagctggactgcattgaaggcgaggctgccccttggatcaagcagaaaacaagagaaagaaagaacgttccctttggggatagtctg

SEQ ID NO: 31 - DNA, 455 bp, Artificial Sequence
  Synthetic construct KIAA0513
cactctgggaggtgcagccctaaggggtggactccagatctccctgcaagagacagcttggcttggctttggctgttggggaggagtccctgccatcccggtgagcctggggctgttgcttagggtcttctgggtggacacgtggagaaagagaaggcaaacgttggaacactaggaaaagctagaaattcagacaacacacatggatccccttaaaacatgtaaatgtgtcagaacacggttgacctgccgccttcttgaacctggtggcccccgttggaactatcagtggcgtctcccatgcacacgccctctgctttctctttcctagactcgcggtgctcacatccagacattaccttgttggtagcccccaagtggcgtgcagtgacaccagtatcttctctgttgcatttttgcaatcttgtgtcccgctcggtgatgttctacaaact

SEQ ID NO: 32 - DNA, 243 bp, Artificial Sequence
  Synthetic construct ABCG1
gagcgattccattttatgactgttgtttttcacattttcatctttctaaggtgtgtctcttttccaatgagaagtcatttttgcaagccaaaagtcgatcaatcgcattcattttaagaaattatacctttttagtacttgctgaagaatgattcagggtaaatcacatactttgtttagagaggcgaggggtttaacccgagtcacccagctggtctcatacatagacagcacttgtgaagg

SEQ ID NO: 33 - DNA, 503 bp, Artificial Sequence
  Synthetic construct HLA-F
cagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgccttctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtcactgacagtgcccagggctctggggtgtctctcacagctaataaagtgtgagacagcttccttgtgtgggac

SEQ ID NO: 34 - DNA, 153 bp, Artificial Sequence
  Synthetic construct BTN3A3
ggatgtttttaatcccactatggactcagtctcctggaaataggtctgtccactcctggtcattggtggatgttaaacccatattcctttcaactgctgcctgctagggaaaactgctcctcattatcatcactattattgctcaccactgta

SEQ ID NO: 35 - DNA, 448 bp, Artificial Sequence
  Synthetic construct WRN
ctggcatcttaaatcagccttccgcaattcatgtagtttctgggtcttctgggagcctacgtgagtacatcacctaacagaatattaaattagacttcctgtaagattgctttaagaaactgttactgtcctgttttctaatctctttattaaaacagtgtatttggaaaatgttatgtgctctgatttgatatagataacagattagtagttacatggtaattatgtgatataaaatattcatatattatcaaaattctgttttgtaaatgtaagaaagcatagttattttacaaattgtttttactgtcttttgaagaagttcttaaatacgttgttaaatggtattagttgaccagggcagtgaaaatgaaaccgcattttgggtgccattaaatagggaaaaaacatgtaaaaaatgtaaaatggagaccaattgcactaggca

SEQ ID NO: 36 - DNA, 288 bp, Artificial Sequence
  Synthetic construct IL27RA
tgccagccaggctagagggatgctcatgcaggttgcaccccagtcctggattagccctcttgatggatgaagacactgaggactcagagaggctgagtcacttacctgaggacacccagccaggcagagctgggattgaaggacccctatagagaagggcttggcccccatggggaagacacggatggaaggtggagcaaaggaaaatacatgaaattgagagtggcagctgcctgccaaaatctgttccgctgtaacagaactgaatttggaccccagcacagtggc

SEQ ID NO: 37 - DNA, 473 bp, Artificial Sequence
  Synthetic construct HCP5
tgaaggatggtgactgcgccatggcctggatctgctgcagtgtcctttcctgtggaggctccactcaaagctggcatcctcctatgtcacctagagtgtgggtcaaagcaatacacctacatgtagaatgtgatgtcagaactcaaacaggctcaccaggcagtgtgcttcttccttgcatgaggatgcaagatgcaacagtttgtcttcacattggaaggacacccctggatgcccctaaccactagacctgtaaaacttcactgcagtggccacttctgaatctctgtaaggtttatttatcttcacccctctggagagaagatgttttaccaaagcctctagtgtaccgtcctcctcttactcatccatcccagtcaacatgatgttgtcaatgaaataaaggaatttaatattctatagtatatccaggttctccagatctcttaagactgtactatagaggcctgggg

SEQ ID NO: 38 - DNA, 575 bp, Artificial Sequence
  Synthetic construct HOXC4
  misc_feature (61)..(61): n is a or t or c or g
  misc_feature (137)..(137): n is a or t or c or g
  misc_feature (210)..(210): n is a or t or c or g
  misc_feature (322)..(322): n is a or t or c or g
gttgtcttagcatggtacctgctgggtgtttttttttaaaaggccattttggggggttatntattttttaagaaaaaaagctgcaaaaattatatattgcaaggtgtgatggtctggcttgggtgaatttcaggggnaaatgaggaaaagaaaaaaggaaagaaattttaaagccaattctcatccttctcctcctcctccttccccccntctttccttaggccttttgcattgaaaatgcaccaggggaggttagtgagggggaagtcattttaaggagaacaaagctatgaagttcttttgtattattgttggggggggntgtgggaggagagggggcgaagacagcagacaaagctaaatgcatctggagagcctctcagagctgttcagtttgaggagccaaaagaaaatcaaaatgaactttcagttcagagaggcagtctataggtagaatctctccccacccctatcgtggttattgtgtttttggactgaatttacttgattattgtaaaacttgcaataaagaattttagtgtcgatgtgaaatgccccgtgatcaataataaacc

SEQ ID NO: 39 - DNA, 537 bp, Artificial Sequence
  Synthetic construct CBFB
gccgctcggaaatcgcttttgtggccacaggaaccaatctgtctctccagttttttccggccagctggcagggagaacagcgacaaacacctagccgagagtatgtcgacttagaaagagaagcaggcaaggtatatttgaaggctcccatgattctgaatggagtctgtgttatctggaaaggctggattgatctccaaagactggatggtatgggctgtctggagtttgatgaggagcgagcccagcaggaggatgcattagcacaacaggcctttgaagaggctcggagaaggacacgcgaatttgaagatagagacaggtctcatcgggaggaaatggaggcaagaagacaacaagaccctagtcctggttccaatttaggtggtggtgatgacctcaaacttcgttaattaatagcacagcagatgtgtgctgcccatctttacatacacattgcttctagttggcagaaataattgattaaaagaccagaaactgtgataactggaggtactacggtctatttctcaacct

SEQ ID NO: 40 - DNA, 111 bp, Artificial Sequence
  Synthetic construct DZIP3
gcattagaccatggttgatgcaacaggggacatgtccaacgtgcagactccacgttttgctaccagaagaattccctggtcaccccagccggcagttgcccaagatctgat

SEQ ID NO: 41 - DNA, 517 bp, Artificial Sequence
  Synthetic construct HLA-B
gtggcggagcagctgagagcctacctggagggcgagtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagctcaggtggaaaaggagggagctactctcaggctg

SEQ ID NO: 42 - DNA, 472 bp, Artificial Sequence
  Synthetic construct HNRNPR
tggatatggctaccctccagattactacggctatgaagattactatgatgattactatggttatgattatcacgactatcgtggaggctatgaagatccctactacggctatgatgatggctatgcagtaagaggaagaggaggaggaaggggagggcgaggtgctccaccaccaccaagggggaggggagcaccacctccaagaggtagagctggctattcacagaggggggcacctttgggaccaccaagaggctctaggggtggcagagggggtcctgctcaacagcagagaggccgtggttcccgtggatctcggggcaatcgtgggggcaatgtaggaggcaagagaaaggcagatgggtacaaccagcctgattccaagcgtcgtcagaccaacaaccaacagaactggggttcccaacccatcgctcagcagccgcttcagcaaggtggtgactattctggtaac

SEQ ID NO: 43 - DNA, 509 bp, Artificial Sequence
  Synthetic construct HLA-B
gacctggcagcgggatggcgaggaccaaactcaggacaccgagcttgtggagaccagaccagcaggagacagaaccttccagaagtgggcagctgtggtggtgccttctggagaagagcagagatacacatgccatgtacagcatgaggggctgccgaagcccctcaccctgagatgggagccatcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtccagcgacagtgcccagggctctgatgtgtctctcacagcttgaaaagcctgagacagctgtcttgtgagggactgagatgcaggatttcttcacgcctcccctttgtgacttcaagagcctctggcatctctttctgcaaaggcacctgaatgtgtctgcgtccctgttagcataatgt

SEQ ID NO: 44 - DNA, 484 bp, Artificial Sequence
  Synthetic construct ATP2B1
gctgaaagggagttgcggcgtggccaaatcttgtggtttagaggtctgaacagaatccaaacacagattcgagtggtgaatgcatttcgtagttctttatatgaagggttagaaaaaccggaatcaagaagttcgattcacaactttatgacacatcctgagtttaggatagaagattcagagcctcatatcccccttattgatgacactgatgccgaagatgatgctcctacaaaacgtaactccagtcctccaccctctcccaacaaaaataacaatgctgttgacagtggaattcaccttacaatagaaatgaacaagtctgctacctcttcatccccaggaagcccactacatagtttggaaacatcactctgattgtaagctgaatgttaacacactagctgcattgtaaagaaacaaattgaaactgggtcttttcacatattgtgatggacaagctagtattcttgtctttggactt

SEQ ID NO: 45 - DNA, 504 bp, Artificial Sequence
  Synthetic construct HLA-G
  misc_feature (61)..(61): n is a or t or c or g
tggaccgcagcggacactgcggctcagatctccaagcgcaagtgtgaggcggccaatgtgngctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactgg

SEQ ID NO: 46 - DNA, 482 bp, Artificial Sequence
  Synthetic construct ABCG1
ggacttcatcgtactcgggattttcttcatctccctccgcctcattgcctattttgtcctcaggtacaaaatccgggcagagaggtaaaacacctgaatgccaggaaacaggaagattagacactgtggccgagggcacgtctagaatcgaggaggcaagcctgtgcccgaccgacgacacagagactcttctgatccaacccctagaaccgcgttgggtttgtgggtgtctcgtgctcagccactctgcccagctgggttggatcttctctccattcccctttctagctttaactaggaagatgtaggcagattggtggtttttttttttttttaacatacagaattttaaataccacaactggggcagaatttaaagctgcaacacagctggtgatgagaggcttcctcagtccagtcgctccttagcaccaggcaccgtgggtcctggatggggaactgcaagcagcctctcagctgat

SEQ ID NO: 47 - DNA, 459 bp, Artificial Sequence
  Synthetic construct nd
tagacaccaaaccagaagaattaacagaagatgacttgatggagatgactgcttccaaaccactgccagacagtgaggaagaagacatagaaaaagagaaaaagcgtgcaagaaaacagattgacactagacagtctgacagaaggggtctgattattcaagactgcttttcacttcgtttgcaacgtggaccgtttggtgatatgagaactgaaactaaagcaaatggtggaagaaggattggtaccatatggaaacaatttagagaacaaaaaagcagaaaagtcagaaattacattgtatttccataaagttacaccaagtatgcctgcctctccagcctccccttttaccttctccacctctgctaaccctgagacagcaagaccaacccctcttctacctattcaatgtaaagacaaggatgaagacttttatgatgatcgactttcacttaat

SEQ ID NO: 48 - DNA, 482 bp, Artificial Sequence
  Synthetic construct HLA-G
caccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaagcagtgacagtgcccagggctctaatgtgtctctcacggcttgtaaattgtgaaacagctgccctgtgtgggactgagt

SEQ ID NO: 49 - DNA, 260 bp, Artificial Sequence
  Synthetic construct UBE3A
atttttttgcgtgaaagtgttacatattctttcacttgtatgtacagagaggtttttctgaatatttattttaagggttaaatcacttttgcttgtgtttattactgcttgaggttgagccttttgagtatttaaaaaatatataccaacagaactactctcccaaggaaaatattgccaccatttgtagaccacgtaaccttcaagtatgtgctacttttttgtccctgtatctaactcaaatcaggaactgtattttt

SEQ ID NO: 50 - DNA, 591 bp, Artificial Sequence
  Synthetic construct HLA-C
gaggcggagcagctgagagcctacctggagggcgagtgcgtggagtggctccgcggatacctggagaacgggaaggagacgctgcagcgcgcggaacgcccaaagacacacgtgacccaccatcccgtctctgaccatgaggccaccctgaggtgctgggccctgggcttctaccctgcggagatcacactgacctggcagcgggatggggaggaccaaactcaggacaccgagcttgtggagaccaggccagcaggagatggaaccttccagaagtgggcagctgtggtggtgccttctggacaagaacagagatacacgtgccatgtgcagcacgaggggctgcaggagccctgcaccctgagatggaagccgtcttcccagcccaccatccccaacttgggcatcgtttctggcccagctgtcctggctgtcctggctgtcctggctgtcctagctgtcctaggagctgtggtcgctgctgtgatacataggaggaagagctcaggtggaaaaggagggagctgctctcaggctgcgtccagcaacagtgcccagggctctgatgagtctctcatcgcttgtaaagcc

SEQ ID NO: 51 - DNA, 544 bp, Artificial Sequence
  Synthetic construct CD59
  misc_feature (425)..(425): n is a or t or c or g
gatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccanagtggccataccaaagaggctaccgattccttcctgctacctggatccctgaagttgccctggtctctgcaccttctaaacctagttcttaagagctttccattacatgagctgtctca

SEQ ID NO: 52 - DNA, 577 bp, Artificial Sequence
  Synthetic construct STAT5B
ctggctcactttgcagttggcactgggttggggaggaagagagctgatgagtgtggcttccctgagctggggtttccctgcttgtccagttgtgagctgtcctcggtgttaccgaggctgtgcctagagagtggagatttttgatgaaaggtgtgctcgctctctgcgttctatcttctctctcctccttgttcctgcaaaccacaagataaaggtagtggtgtgtctcgaccccatcagcctctcacccactcccagacacacacaagtcctcaaaagtttcagctccgtgtgtgagatgtgcaggttttttctagggggtagggggagactaaaatcgaatataacttaaaatgaaagtatactttttataatttttctttttaaaacttggtgaaattatttcagatacatattttagtgtcaaggcagattagttatttagccaccaaaaaaaagtattgtgtacaatttggggcctcaaatttgactctgcctcaaaaaaaagaaatatatcctatgcagagttacagtcacaaagttgtgtattttatgttacaataaagccttcctctga

SEQ ID NO: 53 - DNA, 417 bp, Artificial Sequence
  Synthetic construct RCOR1
acctgtcttacctgtagtaaagcacaattgcagtggcgtcgcattcagaagaagggaaggtcagcagaggctatgcatgttgtgtgatgatgagtgtttacagccaccttctcctaaaacgaaatttataccggggtggatagtattccattaggtagacttatcgactttgctaagtgctttttagacagcttaaaaaattttcaagattttaaaagatgtataaggttaagtttgcaaatataatggaaatgctgtatatcttttgaagtgatgaaatccacgttggaattttaaagaaaatatgttgtaataatgctgttgtaagtaatattttaatgtctctttgcctgttttctatttcagcacattcattgtggtgaatgttcatagcattataactgcttagccattgaa

SEQ ID NO: 54 - DNA, 538 bp, Artificial Sequence
  Synthetic construct BTN3A2
  misc_feature (179)..(179): n is a or t or c or g
gacagctccaggatcgagatcactgtgagtggttgtggagttaagacccctatggactccttcccagctgattatcagagccttagacccagcactccttggattggctctgcagagtgtcttggttgagagaataacgttgcagttcccacagggcatgtgactttgaaagagactanaggccacactcagttaataatggggcacagatgtgttcccacccaacaaatgtgataagtgatcgtgcagccagagccagccttccttcagtcaaggtttccaggcagagcaaataccctagagattctctgtaatattggtaatttggatgaaggaagctagaagaattacagggatgtttttaatcccactatggactcagtctcctggaaaaggatctgtccactcctggtcattggtggatgttaaacccatattcctttcaactgctgcctgctagggaaaactgctcctcattatcatcactattattgctcaccactgtatcccctctactgggcaagtgcttgtcaagttc

SEQ ID NO: 55 - DNA, 513 bp, Artificial Sequence
  Synthetic construct CSTF2T
  misc_feature (59)..(59): n is a or t or c or g
gctactacgactttattttttcttgaaaataaagccttgaggatgtgaacataaatgtnggtatcataaaacaagtagtaataaaagcataaattatcaaatatcaggtgatcttactatggagaagaaaaaccaatatctagttacagatgaaaaaatgtttaatacaaagagctgaaaatgtagaaattgagttatcttccctttgcaaaatttttacactttaaattagtaattctttgtttgcaaggttttcatccctatgctgttgttttctactacctgttttactcttgttcatactgctactttcccatgtttcctatatgcctcccctatattggagaggataactcgcgtttacatatgtgatagttatgcttaatactaattcacagagttggctcgatcattttgagattctagttgctttatctcaaagctttcactgaagggctgtaactgaaaattattaaatgttagtctttagtcaaatctctgcattgtttcatg

SEQ ID NO: 56 - DNA, 527 bp, Artificial Sequence
  Synthetic construct SET
  misc_feature (137)..(137): n is a or t or c or g
  misc_feature (147)..(147): n is a or t or c or g
aaactgcaaggatgtctgtcaaatatcacaattaaacatgccaaaggagaagccatgttgtcaaaatgcccacttaacccacccaaacatctcaaacccaccctttgctgaccttctataaccccatttttttaagntttttttttntttttttaaacaagagaaagtagacagatacatgttggtaaatgctaactgtccatattcacatagagacacagtgtactctctgagcccaatatacagagaaaggaggaaaaaagctagaattctatgcactactacacaggggcctagcaccctccagcttccagcagagcgaagggagcaggtttttcttttttcccacagagctcggtggtgttgattccatacagtttttgttcagacaggaagggataaaaatgaacttcgaacagaaaggggtagagactcttttcccattgtattctgctcaaggtatttccccccaaataaattgagaaccatggagtagagaaaagagacctcaagaacagggcgactga

SEQ ID NO: 57 - DNA, 539 bp, Artificial Sequence
  Synthetic construct KNOP1
ggccacttggagttcctcagtggggaaaagaaaaataagaagtcacctctagccatgtcccatgcctctggggtgaaaacctccccagaccctagacagggtgaggaggaaaccagagttggcaagaagctcaaaaaacacaagaaggaaaaaaagggggcccaggaccccacagccttctcggtccaggacccttggttctgtgaggccagggaggccagggatgttggggacacttgctcagtggggaagaaggatgaggaacaggcagccttggggcagaaacggaagcggaagagccccagagaacacaatgggaaggtgaagaagaaaaaaaaaatccaccaggagggagatgccctcccaggccactccaagccctccaggtccatggagagcagccctaggaaaggaagtaaaaagaagccagtcaaagttgaggctccggaatacatccccataagtgatgaccctaaggcctccgcaaagaaaaagatgaagtccaaaaagaaggtagagcagccagtcatcgaggagcc

SEQ ID NO: 58 - DNA, 530 bp, Artificial Sequence
  Synthetic construct CLIC5
aagtgtgcataatttcatttaacgttaaagaaatagatccaattcctttcttgcaaccaaaaataaataaaatacgttgcctcaatataaggtttgggctattctgtgtttctatagaagcaatctgtttttggtaaaatgtacttttaaggatccagtcatctgaagtattttatgtagagttagagatttcacaatattgactatacatatatttaaaatataaattatccagctgatgtttgaatttgtcttactttcctggccacctcgttgtcctattttataagctggggagttaactagcttaacaaaagatgcttagcttttgtaaaagaacaagtgtttcattttacaaagacactccaaatgatagttacttgattttctcgagacctttaactatggtgatgaataacaggacttgctttcaagccttaataaatgtaaaatgccttttaatgaagatacagctgagtgttttcctcatgaatctgaaccaattaccaatttgtgttccagtcttgatt

SEQ ID NO: 59 - DNA, 544 bp, Artificial Sequence
  Synthetic construct VAMP4
gaagccacaaagatgccacatgttagtatatcagtgagaggtgactccacagtgctctctggagaagcaatatgagtgactgaagagtggggccttttgcttttgcctggatataggggtgctcttctactgtaattgggtgtggaaaaactctggctttatggtattccattaggttcttttcatttaaagtagtcttaaaatcaaagtatccaatattttaaagccacaaagtagattacataattagcagagattttagtcagtaaaatgttagaaatcaaactataagaaaattcaagtcctttattttgtgtcttgggtatatgtcattattttaaattccacactcccttatttaatcactttggtaagtgcctttgatgttttgaaatgtatagtgggagatgagcaaatgtaaatgtcatgtgccctgttccctagcttctcaattcctcataaccatttttaccagtgttgcaaagtttagacctttgtgttaatatcagaagtgtatttgtagcccctccatagtgaacaatga

SEQ ID NO: 60 - DNA, 558 bp, Artificial Sequence
  Synthetic construct HLA-A
  misc_feature (126)..(126): n is a or t or c or g
  misc_feature (127)..(127): n is a or t or c or g
  misc_feature (130)..(130): n is a or t or c or g
  misc_feature (139)..(139): n is a or t or c or g
  misc_feature (144)..(144): n is a or t or c or g
  misc_feature (148)..(148): n is a or t or c or g
  misc_feature (151)..(151): n is a or t or c or g
  misc_feature (168)..(168): n is a or t or c or g
  misc_feature (176)..(176): n is a or t or c or g
  misc_feature (177)..(177): n is a or t or c or g
  misc_feature (220)..(220): n is a or t or c or g
  misc_feature (221)..(221): n is a or t or c or g
  misc_feature (251)..(251): n is a or t or c or g
  misc_feature (392)..(392): n is a or t or c or g
  misc_feature (426)..(426): n is a or t or c or g
gacagacctcaggagggctattggtccaggacccacacctgctttcttcatgtttcctgatcccgccctgggtctgcagtcacacatttctggaaacttctctggggtccaagactaggaggttcnnctnggaccttanggccntggntcntttctggtatctcacanggacattnncttctcacagatagaaaaggagggagttacactcaggctgcanncagtgacagtgcccaggctctgatgtgtcnctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcntaatgtgaggaggtggggagaccaccccaccccnatgtccaccatgaccctcttcccacgctgacctgtgctccctccccaatcatctttcctgttccagagaggtggggctgaggtgtctccatctctgtctcaacttcatggtgcactgagctgtaacttcttc

SEQ ID NO: 61 - DNA, 498 bp, Artificial Sequence
  Synthetic construct N4BP2L2
  misc_feature (169)..(169): n is a or t or c or g
  misc_feature (176)..(176): n is a or t or c or g
  misc_feature (178)..(178): n is a or t or c or g
  misc_feature (179)..(179): n is a or t or c or g
  misc_feature (180)..(180): n is a or t or c or g
  misc_feature (183)..(183): n is a or t or c or g
  misc_feature (186)..(186): n is a or t or c or g
  misc_feature (187)..(187): n is a or t or c or g
  misc_feature (188)..(188): n is a or t or c or g
  misc_feature (189)..(189): n is a or t or c or g
  misc_feature (192)..(192): n is a or t or c or g
  misc_feature (194)..(194): n is a or t or c or g
  misc_feature (195)..(195): n is a or t or c or g
  misc_feature (196)..(196): n is a or t or c or g
  misc_feature (197)..(197): n is a or t or c or g
  misc_feature (199)..(199): n is a or t or c or g
  misc_feature (200)..(200): n is a or t or c or g
  misc_feature (431)..(431): n is a or t or c or g
  misc_feature (433)..(433): n is a or t or c or g
tctgctgtcttcatggtactgctgaagatcatgatcacggagaaaagtcagagtgctcagtgccaacccaagggattctttccagagacgtacccgttggataccaaaattagtttggataatctgttcaaccattcttgataagttatctgaataataaaaaaactcnacagagnannnaanaannnnaanannnnanngtctaatttgaagatgtatgccatactttgtttgatagaagaaagtaaggcacagaaaacttgagtaccttatttttaaaactgcattaggattaaaaggttagcccctatatccaagtattggtctgagatcccatttctagaattctgaaatccaaaaagctctgaaaatcaatagttatttttccaaatgtattcattgtggtaaaatacaccaatataaaatttacnancttaaccacttttaagtgtgttataaatacattcatgctaccatcactagcatccatctctggaa

SEQ ID NO: 62 - DNA, 516 bp, Artificial Sequence
  Synthetic construct HLA-A
  misc_feature (64)..(64): n is a or t or c or g
  misc_feature (281)..(281): n is a or t or c or g
  misc_feature (282)..(282): n is a or t or c or g
  misc_feature (322)..(322): n is a or t or c or g
tgagatgggagctgtcttcccagcccaccatccccatcgtgggcatcattgctggcctggttcnccttggagctgtgatcactggagctgtggtcgctgccgtgatgtggaggaggaagagctcaggtggagaaggggtgaaagatagaaaaggagggagttacactcaggctgcaagcagtgacagtgcccagggctctgatgtgtctctcacagcttgtaaagtgtgagacagctgccttgtgtgggactgagaggcaagagttgttcctgcccttccnnttgtgacttgaagaaccctgactttgtttctgcaaaggcncctgcatgtgtctgtgttcgtgtaggcataatgtgaggaggtggggagaccaccccaccccgatgtccaccatgaccctcttcccacgctgacctgtgctccctctccaatcatctttcctgttccagagaggtggggctgaggtgtctccatctctgtctcaacttcatggtgcactgagctgtaacttcttc

SEQ ID NO: 63 - DNA, 523 bp, Artificial Sequence
  Synthetic construct HLA-J
  misc_feature (82)..(82): n is a or t or c or g
  misc_feature (102)..(102): n is a or t or c or g
  misc_feature (107)..(107): n is a or t or c or g
  misc_feature (108)..(108): n is a or t or c or g
  misc_feature (134)..(134): n is a or t or c or g
  misc_feature (323)..(323): n is a or t or c or g
  misc_feature (426)..(426): n is a, c, g, or t
  misc_feature (427)..(427): n is a or t or c or g
  misc_feature (428)..(428): n is a or t or c or g
  misc_feature (429)..(429): n is a or t or c or g
  misc_feature (430)..(430): n is a or t or c or g
  misc_feature (431)..(431): n is a or t or c or g
  misc_feature (432)..(432): n is a or t or c or g
  misc_feature (433)..(433): n is a or t or c or g
  misc_feature (434)..(434): n is a or t or c or g
  misc_feature (435)..(435): n is a or t or c or g
  misc_feature (436)..(436): n is a or t or c or g
  misc_feature (437)..(437): n is a or t or c or g
  misc_feature (438)..(438): n is a or t or c or g
  misc_feature (439)..(439): n is a or t or c or g
  misc_feature (440)..(440): n is a or t or c or g
tacctggagggcacctgcatggagtggctccgcagacacctggagaacgggaaggagacgctgcagcgcgcggacccccccnaagacacacgtgacccaccnccctnnctctgaacatgaggcataacgaggtnctgggttctgggcttctaccctgcggagatcacattgacctggcagcgggatggggaggaccagacccaggacatggagctcgtggagaccaggcccacaggggatggaaccttccagaagtgggcggttgtggtagtgccttctggagaggaacagagatacacatgccatgtgcagcacaaggggcntgcccaagcccctcatcctgagatgggagccctctccccagcccaccatccccattgtgggtatcattgctggcctggttctccttggagctgtggtcactgnnnnnnnnnnnnnnnctgtgatgtggaggaagaagagctcagatagaaaaggagggagctactctcaggctgcaagcagccaaagtgcccagggctct

SEQ ID NO: 64 - DNA, 531 bp, Artificial Sequence
  Synthetic construct NDFIP1
ccatatttgctgttcaagttaatctagaaatttattcaattctgtatgaacacctggaagcaaaatcatagtgcaaaaatacatttaaggtgtggtcaaaaataagtctttaattggtaaataataagcattaattttttatagcctgtattcacaattctgcggtaccttattgtacctaagggattctaaaggtgttgtcactgtataaaacagaaagcactaggatacaaatgaagcttaattactaaaatgtaattcttgacactctttctataattagcgttcttcacccccacccccacccccaccccccttattttccttttgtctcctggtgattaggccaaagtctgggagtaaggagaggattaggtacttaggagcaaagaaagaagtagcttggaacttttgagatgatccctaacatactgtactacttgcttttacaatgtgttagcagaaaccagtgggttataatgtagaatgatgtgctttctgcccaagtggtaattcatcttggtttgctat

SEQ ID NO: 65 - DNA, 500 bp, Artificial Sequence
  Synthetic construct MRPL42
aagcatcttgaaacaccatggctgtagctgcagtaaaatgggtgatgtcaaagagaactatcttgaaacatttatttccagtccaaaatggagctttatattgtgtttgtcataaatctacgtattctcctctaccagatgactataattgcaacgtagagcttgctctgacttctgatggcaggacaatagtatgctaccacccttctgtggacattccatatgaacacacaaaacctatccctcggccagatcctgtgcataataatgaagaaacacatgatcaagtgctgaaaaccagattggaagaaaaagttgaacaccttgaggaaggacctatgatagaacaacttagcaaaatgttctttactactaagcaccgttggtatcctcatggacggtatcacagatgtcgtaaggaatcttgaatcctccaaaagacagatgatgcggaggttcctgggggaatcaaagagaaatgtgcctcatttgccatttga

SEQ ID NO: 66 - DNA, 505 bp, Artificial Sequence
  Synthetic construct RSF1
aacaaatcctgaagtctttctgtgaagtgaccagttctgaactttgaagataaataattgctgtaaattccttttgattttctttttccaggttcatggtccttggtaatttcattcatggaaaaaaatcttattataataacaacaaagatttgtatatttttgactttatatttcctgagctctcctgactttgtgaaaaagggtggatgaaaatgcattccgaatctgtgagggcccaaaacagaatttaggggtgggtgaaagcacttgtgctttagctttttcatattaaatatatattatatttaaacattcatggcatagatgatgatttacagacaatttaaaagttcaagtctgtactgttacagtttgagaattgtagataacatcatacataagtcatttagtaacagcctttgtgaaatgaacttgtttactattggagataaccacacttaataaagaagagacagtgaaagtaccatcataattaacctaa

SEQ ID NO: 67 - DNA, 494 bp, Artificial Sequence
  Synthetic construct ACSL5
acaattcatgttttctagccactccacaaaccactaaaattttagttttagcctatcactcatgtcaatcatatctatgagacaaatgtctccgatgctcttctgcgtaattaaattgtgtactgaagggaaaagtttgatcataccaaacatttcctaaactctctagttagatatctgacttgggagtataaaaattggtctatgacatattgtccaaaaggaatgctgttcttaaagcattatttacagaaggaactggggagtaaatctgtccctacagtttgctgctgagctggaagctgtgggggaaggagttgacaggtgggcccagtgaacttttccagtaaatgaagcaagcactgaataaaaacctcctgaactgggaacaaagatctacaggcaagcaagatgcccacacaacaggcttattttctgtgaaggaaccaactgatctcccccacccttggattagagttcctgctctaccttac

SEQ ID NO: 68 - DNA, 453 bp, Artificial Sequence
  Synthetic construct FBXO3
gaaactatgtgactcattctgtgaaaagacttcttgcagttgtgagttatttagaaatgatcaaaatttgtaattaggctaatccatttagtgattcctaatattttgtactcacagagaactaattgactaaacaacttgaacgctagtggtttgtccttagacaatctgtctttgaatttaaagtctttatcgctaagaccttgactttaaatttttcatcactacaaccttgaatttaatttcaggtcttcaacatgatgaccttggatttaatttaaagtcttcaacactatgcgctttatcatattattcacagatgcatttttgaaatgtagtatgtaaaagtatgtaacgtgctgtttattaacaaaagattgttcacaacatctcatgtagtttaaatttgtaaatactgcttctgttttgtttctcctttatacacttgactgt

SEQ ID NO: 69 - DNA, 436 bp, Artificial Sequence
  Synthetic construct ZNF302
gaaaaatctgtgtacatgtagcaaatgtgggaaagactataggcaataggaatctcctgcaaactcctacaggagaaaagttgtatgaatgtggaaactttagaaattgaaggaatttttccagttccaagtgcatcccttattctataggaaataaactggagacaaatctcatttaagagatgcagcaaagtgttcactaagagtgtttatcttgccagacataagaagatgaatggtagagcaacctgaaggatttagaaattacatataaatctttgcagttatgctatttgtaaacaggattatataggagagcaaataaacataagtatgcatttcttagagcagtagcttgcagtttcagttgagttctacttagaaattctttttagctagtgggcatgtgaagatatttagtcacccagaggagcca

SEQ ID NO: 70 - DNA, 527 bp, Artificial Sequence
  Synthetic construct ECHDC2
ttcacgggccgacgactgagtggaactgaggcccacgtactggggctggtgaatcacgctgtggcccagaacgaggagggggacgccgcctaccagcgggcacgagcactggcccaggagatcctgccccaggcccccattgccgtgcggctgggcaaagtagccattaaccgaggaacggaggtggacattgcatctgggatggccattgaagggatgtgctatgcccagaatattccaacccgggaccggctagagggcatggcagccttcagggagaagcggactcccaaatttgttggcaaatgacccccattttaaccttcagcatgggagatgcatgccctgaagagcaggatccagaaggaagatttgtggccagattgccttcatcatttcacctctccagacttccatttcttcacaaggatgatgatggaaataaaatgactggcgtgatgcctggaaccaaggtgctgatcctaccacctactgctaccttccttagcttcaccctggctagaaat

SEQ ID NO: 71 - DNA, 510 bp, Artificial Sequence
  Synthetic construct ARHGAP15
gtgggaggacatccacgttgtcaccggagcactgaagatgtttttccgggagctgcctgagccgctcttcccttacagtttctttgagcagtttgtggaagcgatcaaaaagcaagacaacaacacaagaattgaagctgtaaaatctcttgtacaaaaactccctccgccaaatcgtgacaccatgaaagtcctctttggacatctaactaagatagtgccaaagcctccaagaacctcatgtccacgcaaagcttggggattgtatttggacctacccttctgcgagctgaaaatgaaacaggaaacatggcgatccacatggtctaccagaaccagatagctgagctcatgctgagtgagtacagtaagatcttcggctcagaggaagactgacagacaagacaagctactgaatacgttcacatctgtcttgatgcctaatatttttacatttctgtaaacatatttctgaaatattttttgcctttcaagcgacagatgcctcat

SEQ ID NO: 72 - DNA, 528 bp, Artificial Sequence
  Synthetic construct NAP1L2
agtgttgtgtagcttaatccttctgaagtctttttgtcatgtagctattaatctgtggctatgaaatgatcagaaatgctaagtgagatcaatatttgtttggaaaaaaaatcttgggaaacaacccaagggttttcgctgttgttgtttttctttttctatttttgtttacttagtcctttagctagtggatttaattttgttgtgcctgcttcattttgcaataacaatgcagtagaatttaaaacttggatgcttaagaggcctgcatatagataagaatttcaggcaaaactacatttattgttaataacagcttgttcataggctcttgtattttatgtaactgtgataaataatgaaacttagttatattgaggttattgtttgtcggtgaagtgttagtcacagtattttcaaaagtttgcacatattgttctgtgtaattgtgtaagccataattacagtgtttaattctcttttcctattacatcattcattgaaagtgatcactttaccattttgaaa

SEQ ID NO: 73 - DNA, 484 bp, Artificial Sequence
  Synthetic construct CCNJ
aacaacagccagtgcctgtggtaacttaatgtcttgtcaaatacttttattgattggtttatatgccattcttgttatagaagaatatgccttttaaaaaagcttattaataacactttcccaatttatattttaaaaagctaaagaacactggattaataatcttttgggagggtagaataaaataattgattactattgctgcatacccggggtgggatggggtggttggagaaccagaactatttttaaaacattaggtttcaatataaatacaactcacaactgctagctttggggggtgggggaacattgtttgggttttgttttgtttaatttattgattagtctttaaagtaggcttttttttttttttttgagaatactggaccatcattaaatgtgtactgtgaagagattaatatgtatgaagggctttaccaaagtccactaaataaacactactcaagtacagactgcaaac

SEQ ID NO: 74 - DNA, 463 bp, Artificial Sequence
  Synthetic construct SINHCAF
agggagaaacatttgcctgccttgtaataacaagactcagtgcttattttttaaactgcattttaaaaattggatagtataataacaataaggagtaagccaccttttataggcaccctgtagttttatagttcttaatctaaacattttatatttccttcttttggaaaaaacctacatgctacaagccaccatatgcacagactatacagtgagttgagttggctctcccacagtctttgaggtgaattacaaaagtccagccattatcatcctcctgagttatttgaaatgattttttttgtacattttggctgcagtattggtggtagaatatactataatatggatcatctctacttctgtatttatttatttattactagacctcaaccacagtcttctttttccccttccacctctctttgcctgtaggatgtactgtatgtagtcatgcactttg

SEQ ID NO: 75 - DNA, 394 bp, Artificial Sequence
  Synthetic construct PLEKHA8P1
gactgaagccctcttgtggctgaagagaggtctcaaatttttgaagggatttttgacagaagtgaaaaatggggaaaaggatatccagacagccctgaataacgcatatggtaaaacattgcggcaacaccatggctgggtagttcgaggggtttttgcgttagctttaagggcaactccatcctatgaagattttgtggccgcgttaaccgtaaaggaaggtgaccaccggaaagaagctttcagtattgggatgcagagggacctcagcctttacctccctgccatgaagaagcagatggccatactggacgctttataagaggtccatgggctggaatctgatgaggttgtatgatggctgctgggcagcacctcctaacttcagggaata

SEQ ID NO: 76 - DNA, 501 bp, Artificial Sequence
  Synthetic construct GVINP1
gattttgtcattgattgctgtactagtttggttgcaagtgattgtttgttggttttgagggatggcaggaatttcccatttaagaactatcgacaagcaggaggggattatgccatgtggagcatcaccccagatacctccatccagctatactggaaatggtttgtctctcacttcagatcaaatctagaagaaaaatatcagaaaaaatttgcaggtaaaggtaaaatccccaatgcatgggccaaaatcacaaagcaagatgtgcttgaagacttgaaaaaacagtaatactcagtgaccacaaaagagccccagtgtctgaagaaaagaggcagtgtacctgaacaatccaaaaacaactactcattacaattagaaccttcatcatttccatgtttaaaatgaaatgtgtaaaaatcaatcaattaacacattaagagatgaaaatttccagtaaaaggacttcatttctctctgcctcaattcctctagtttaaa

SEQ ID NO: 77 - DNA, 494 bp, Artificial Sequence
  Synthetic construct FAM117A
gagaagacgccacagatttccttccctctcctccaggagaccataagatagatcccccatcctctcagccctattcccatgcctccctctcattggaggagctgaccaaagcagccctaacgggccataacacttgaccaattcagctgctggcagagggaggaaacaagtgttttcccaagtggcattttcatctcgctttcaccctgactaaagattgtcttaagtagcagcccagcccgcccagccccaggtgggtagtggggaggagagctggcattcctccaggtggcaaatggcgactctatactctccgcccgccccagggctggatggattagaaaaatccctatttttcttgtatcgatgtagagactctattttctcccaaagacactatttttgcagctgtttgaagtttgtatattttccgtactgcagagcttacacaaaattgaagaatgttaatgttcgagttttcttatcttgtgttt

SEQ ID NO: 78 - DNA, 513 bp, Artificial Sequence
  Synthetic construct ZNF506
tcctcctttttttgcgtgtgtgatggggtttcgcacttgttgcccaggctggagtgcaatgggtacaatctcggctcactgcaacctccgcctcccaggttcaagtgattcttctgcctcagcctctcaagtaactgggattacaggcatgcgccactaagggaggagaccactcctcatattgtcttatgcccaatttctgcttccaaagaaagaagaagtaaaaactaaaaggcagaaatgaaatccacaggcagacagcccagcgccacaccatgggcctggtagttaaagatcgagccctgacctaatcggttatgttatctacagattacagacattgtatagaaaagcactttgaaaatccctgtcctgttctgttccattctaattactggtgcatgcagccttcagtcacttactccctgcttgctcaatcgatcacgaccctctcatggaaacccccttagagttgtaagcccttaagagggataggaattgctcactcaggga

SEQ ID NO: 79 - DNA, 514 bp, Artificial Sequence
  Synthetic construct BRIP1
aatgttattattcttgagtttttgggtttctttttttgagacagagtcttggtctgttgcccaggctggaatgcagtggtgtgctctgggctcactgcaaccttcacctccaggttcaagtgattctcctgcctcagccttctgagtagctgggactacaggtgtgcaccaccatgcccagctagtttttgtatttttagtagagatggggtttcaccatgttggccaggctggtctcgaactcctggcctcgtgatctgcccttctctgcctccctaagttgctgggattacaggtgtgagccacagtgcctggcccattcttgagttttgataaagtaattcatacaaagtattgtcctcaaataagtcttccttagctaaatgcaatttaaaattattcaaagatcctagggcacttctagtttcacgtaaatattcatattaggtggttctcttcatccatttgttttcacactgatacataaaaattaacagcagtctaatctagtgac

SEQ ID NO: 80 - DNA, 230 bp, Artificial Sequence
  Synthetic construct IL27RA
tcccttgcatccaggtgcagcatggactgagttcttgacaacagaatgtggtcagaagtgacatatgccaacacggggtctgggtgggggctcccccacatcctttccttgcctatgagctggaacataacacatgcctatgatccagctttggtcatacccaaggggaaggtggagcaagaaatgaaaaggaacctgaatccctgaatgactgcatggatagaaccact

SEQ ID NO: 81 - DNA, 467 bp, Artificial Sequence
  Synthetic construct DERL1
agagtttgtgtgtacctctccatatttaatttatatgataaaataggtggggagagtctgaaccttaactgtcatgttttgttgttcatctgtggccacaataaagtttacttgtaaaattttagaggccattactccaattatgttgcacgtacactcattgtacaggcgtggagactcattgtatgtataagaatattctgacagtgagtgacccggagtctctggtgtaccctcttaccagtcagctgcctgcgagcagtcattttttcctaaaggtttacaagtatttagaactcttcagttcagggcaaaatgttcatgaagttattcctcttaaacatggttaggaagctgatgacgttattgattttgtctggattatgtttctggaataattttaccaaaacaagctatttgagttttgacttgacaaggcaaaacatgacagtggattctctttacaa

SEQ ID NO: 82 - DNA, 267 bp, Artificial Sequence
  Synthetic construct SINHCAF
aaaaggacttccagagctgttttggattgcatgagactcgttcaggagacatctgcaatgcctgtgtcctgcttgtgaaaagatggaagaagttgccagcaggatcaaaaaaaaactggaatcatgtggtagatgcaagggctggacccagtctaaagactacattgaaaccaaagaaagtgaaaactctatctgggaacaggataaaaagcaaccagatcagtaaactgcagaaggaatttaaacgtcataattctgatgctcaca

SEQ ID NO: 83 - DNA, 370 bp, Artificial Sequence
  Synthetic construct YPEL3
ggctggaaatatgaacaggcctttgagagcagccagaagtacaaagaggggaagtacatcattgaactcaaccacatgatcaaagacaacggctgggactgacccccgctcccccgacgcatgtggctccagcccggcctggccgccagggagcgccactggcttcccgccacccgaagggagctctggaccctcagagcccctgcagaggacggatctagctcctgtatatatattttattgcatgcactgtgaccttggggggagaacagaagggggacgacgcccccgcacctcctgcgatctggctggcttggatctcgtttttaacccgttcctgccccacctgccctatagttatggcctgtgt

SEQ ID NO: 84 - DNA, 412 bp, Artificial Sequence
  Synthetic construct SLC25A33
tttttggacttatggcagctgctgctctttctaagggctgtgcctcctgcattgcttatccacacgaagtcataaggacgaggctccgggaagagggcaccaagtacaagtcttttgtccagacggcgcgcctggtgttccgggaagaaggctaccttgccttttatagaggactgtttgcccagcttatccggcagatcccaaatactgccattgtgttgtctacttatgagttaattgtgtacctgttagaagaccgtactcagtaacaggccggaaaattgtgctctagaagaataaaactgaaaaactctagagaattttttttccccattgatgtttagaaagtttgagactgaaacaggaaaggccataaaatatctggttcatatcacctgttggacatttcctt

SEQ ID NO: 85 - DNA, 359 bp, Artificial Sequence
  Synthetic construct SENP7
atttcaaaatcatacgaatcggggtcagttaagcctcagtattcttagcttttgttgattttggcactatctttatattattaaatatatttgttgtttggatatttcatataaagatggctataattacatatttcattcccaatttgtgtgtgttggggggtacttttaaaggtgactattgttttgtacatctaattttgggaaaccaagtctataagacatcttgtgatttcttaatgtttttgtttgtatgtttttcaaagatatcactgtcctttatcatgttttgaagattgtttaaaattcattttcctaaattaatgtgcaagtaatgttttgaggatatcggtgtttta

SEQ ID NO: 86 - DNA, 374 bp, Artificial Sequence
  Synthetic construct NSD3
atctgagcagtctaacgaggcccacctcctccaccacagaacgtggctatgttccaagtgctactctcactcagcctgttgcggatcttcatggcctcaggagacttgtttctccatgggctcttctggactgcacacttccaccatagcttgctgggttgatctagatgtctgtttgttgtatggaaattttgggggaaaaaatccaaaacacaaactgtgggttgaaatattaaccgtctccttggttccttggtattcaccgtgcctgatctgcacatttcatcgtggctgtttctgtatagcctatactgcattagcccaagagattgttgctttgtaactttttgcactattgttttggctggatttgt

SEQ ID NO: 87 - DNA, 282 bp, Artificial Sequence
  Synthetic construct MFSD14C
tttggcccaccaagtgtgtaccatgctgctattgtcatcttccttgaattctttgcgtggggccttttgacaactccaatgttgactgttctacatgaaacattttctcaacacacattcctcatgaatggtctcattcaaggtgtaaagggcctgctctcttttttgagtgccccactcattggtgccctgtctgatgtgtgggggaggaagccctttctcctcggcactgtattctttacctgcttcccaatcccactgatgaggatcagcccatgccgg

SEQ ID NO: 88 - DNA, 394 bp, Artificial Sequence
  Synthetic construct PCYT1A
  misc_feature (33)..(33): n is a or t or c or g
aaagagtggtccctagcttctatttttccaagnaaataagtagaacatgttcttgggattataccattaaatgttaattttcttgaagaagaaagattgttgtctgccaagattttatgttagcgctcggattgaggcagaaaacggaagcaccaggtttaacactgggatgacttgggttgtgttcctggaggtttgaagtgggccttcccgccttttgagggggaaaactgactgttttgaacacatatcctggattagttgggttgttttgtattgaggatccctaaaagacaaaagagtcttggaaaggactgctggcttcgtcttgaacgttcgccccatcaccagtgtgagtccgtggggaagtcttcctcaagtggggttcagtctg

SEQ ID NO: 89 - DNA, 398 bp, Artificial Sequence
  Synthetic construct TTC14
  misc_feature (253)..(253): n is a or t or c or g
agaaagatagatgccctctctcttcatcttcacttgaaataccggatgattttggaggtaggtctgaagatccaagagatttttataacagctataaaacccaagcaggtagtagcaaaacagaaaagccatataaatcagaaagacatttttccagtagaagaaattcctcagattccttctgtaggaattcagaggacaagatttatggttataggagatttgaaaaggatatagagggaagaaaagagcnctatagaaggtgggaaccaggttctgtgaggcattctacctcaccagcaagctcagaatactcttggaagtcagttgagaaatacaaaaaatacgctcactctggatcacgtgatttcagtagacatgagcaaagataccgttta

SEQ ID NO: 90 - DNA, 343 bp, Artificial Sequence
  Synthetic construct TTC14
aatacaccaaggatatcggcaccattacacaaaatgccattaagtgaagtttttggttgtattagtcataacagttgagtgcagaaatctctgcttctaaaattatttgtagagattacaggaaacaaatgcttttaagtaagtttttctcaaattttgtttcagttctaggtcccttgtcacagcttggtttttagactttttatgtatatgtttatgtacagtatattactcttgacagtttgaatttcttcacctaaagataattctctgaaagtactgtttcttcattctattgcggtatatgagaattcctgggtgcatcttattctgctgtttgaga

SEQ ID NO: 91 - DNA, 432 bp, Artificial Sequence
  Synthetic construct ARHGAP27
  misc_feature (248)..(248): n is a or t or c or g
  misc_feature (280)..(280): n is a or t or c or g
ggggaaggaggacacaacctcaagaaaggaagcgtgaaccccagggaacagcgggtcccttccctcctcagacacaagccacctcagcttgtggctcttggcccccagccccaccaacccacctgttcatttattcaacagacaatgacagctgatatttattggacatttgcaccatgccaagcattcggcttggattatcccatttgtttctcacagccggtatttattgtctgctcctctgtgcnaggtgctgtgctctgggcaggggcactgcatnggctgcctgccctggtggagcttgtggtctgatgggtgaggctgacccaagcccaccccattgccaacagggccagggcaagagtacacacaggggcctcataccatatgtctaaatatttaaaagttatcaatcaagctaacaactgttaa

SEQ ID NO: 92 - DNA, 474 bp, Artificial Sequence
  Synthetic construct MTMR10
  misc_feature (416)..(416): n is a or t or c or g
ctatgacagccttcagtcgggccagggtgaagctgcttataccacctctgccgtcagagggacatgtggtgacagcagtggtgtggctgcacagggcgcactagagagagctcagcacccctgctgcccgccagcagagcccgtgctgagggaatgccgcacagatgctgatgcactgggtgaaatttctagtattgaacgtaaaggtgtacagtgtcttgctgttattttatgatggaaactgattttgaaaccaaaaatagctaactaactttatttaaggaaaggatattaatttgtactaacagagggtgaaagctgttcacatttgtcaacaaaatctgcttgctgcagtagtaacctcaagtggttaaaacttgatttcccgagaaaactaaaacctttgtgcctaaaantgatgacttgagttcaagtgggatgagcaagaagatgtgttatcttgttgttcaacag

SEQ ID NO: 93 - DNA, 531 bp, Artificial Sequence
  Synthetic construct FAM84B
  misc_feature (103)..(103): n is a or t or c or g
acttctcatgtctttggtgctttggccctaatagcactggacaacaccacgaccacatggaaacatatttttggaagcaaaactttaattttatataacgtangctatggagagctaagacaatttaaggactacttgttttctattttttttcttaataaaatggaatccactgtgttgaagactcttgatatcatgtgcttgtctaaccattttttgttttataaattagaataaaatatagttgtgataatggtcatcgaatggatttgtttggaaagctacatcttatttgtgaaatgttttttaaatcagagtaactatcaactgattcagctttttgttgttttgttcttggctataatacttgtgactcatgaagaattatgttgacaaacaggataaattccacatgcattttatttcccagtgagttgtataaactttatttttgttgaaggttgtatgttaaatcaatgttacattcttatatcacttcttgagaaggaagttccgatttgaaattgtatc

SEQ ID NO: 94 - DNA, 488 bp, Artificial Sequence
  Synthetic construct TRAPPC5
cgcgcaccgaggtgagcctgagcgccttcgcactgctgttctccgagctggtacagcactgccagagccgcgtcttctccgtggccgagctgcagtcgcgcctggccgcgctgggccgccaggtgggcgcgcgcgtgctggatgcgctggtggcgcgcgaaaagggtgcccggcgtgagaccaaggtgctaggcgcgttgctcttcgtcaagggcgccgtgtggaaggcgctcttcggcaaggaggcggacaagctggagcaggccaacgatgacgcgcgcaccttctacatcatcgagcgcgagccgctcatcaacacctacatctccgtgcccaaggagaacagcacgctcaactgcgccagcttcacggcgggcatcgtggaggcggtgctcacacacagcggcttccctgccaaggtcacggcgcactggcacaagggcaccacgctcatgatcaagttcgaggaggcagtcatcgctcgagac

SEQ ID NO: 95 - DNA, 438 bp, Artificial Sequence
  Synthetic construct RDH10
  misc_feature (109)..(109): n is a or t or c or g
gtgtctggagacctaattatcctaaaagatcatacattttctacctatgaattttgctgcatacagaaagtgccctttcctcaggaagttgctgtgtttcatttctttnggatggactcttatctagaatacatagcagctctgcaaagaaacagtttttaaaaatgggaacttctacattgaaaagtccccatttttgtgccaactatgattagtgagaggaagaaatcttattctatggcatatgtatggaagggtgtaaagattcttttgaaaggtttattcacattgtagaacagcaaatgacatttttacagtatttttttgtaaagcaaactattttgtgccttgaatttggtatatgtgtattagtgaaacattgtaaaggtgaacttctacctctgtatctaaatgtataccatccacttgtaaatgact

SEQ ID NO: 96 - DNA, 542 bp, Artificial Sequence
  Synthetic construct FAM160B1
ggaccaaaattcttagctcgcttacactgttgctagtgtaaacattgttacacttaattttacagggcacaaattatggaattacttcataaattcatggtaatgttttcataaattattgcattaacatacaattgccatttaattatgaagtccatcagtattgacagaagacgttacagtgaagtgctaaaaccacactatatggtaattatattttgggttatgtagtaaatcaaatatgcatgtcattgtgacactttatgtgttaaaacatggtagataatcacattttctgggccctgtaaaatagtgttactgtaatactctgttttgcctcctgccttgtttacattaaacaggatatttggtaaatttttttgtattgaaagttgtgtaggttactgacagtgttaccagcgtctggaattcttggtccctccgtggagaaactcttcagatggtcattgtgtacctactctctcttcaaaggaagttgtgatcaagttcaactttttgtgctaacaatgcatgcaggacta

SEQ ID NO: 97 - DNA, 510 bp, Artificial Sequence
  Synthetic construct SLFN5
  misc_feature (333)..(333): n is a or t or c or g
gacaagctgaccaaataggccttagaagggagaggtgcctgggcagttacaaggacctaggtagcaggggtgacttggccacagtcattttctttacttgatttaaggaggttaaattacagtgagagagttgcacttatttagtttgcataccgtactcaactcttagctagcatagggcaggttccttagttgaaaaatttccacaaaacatgatgtaaagtggaaagatcagttcccccaaagaatatcagttaccaaagtaggatgaaaggatgttgggtagatcaaaacaacaaatgtccattacagcaattaacaaaagttagggantggggaagtctgagtgctaatatggatttcaaaacaactttaaaaatggcttcaaacattttatgctttttgtaatttagtcattttaatattagataaatcttcctgtaactgaaagttcttgagttaaagaaaaatattcggtagttcagctcttccttcaattttattagttgc

SEQ ID NO: 98 - DNA, 489 bp, Artificial Sequence
  Synthetic construct C12orf73
  misc_feature (459)..(459): n is a or t or c or g
cgcggtgtagtcgtgagctggaacttctgacactgccccttccttccccgtccagcctgccgaccatgcccgcgggcgtgcccatgtccacctacctgaaaatgttcgcagccagtctcctggccatgtgcgcaggggcagaagtggtgcacaggtactaccgaccggacctgacaatacctgaaattccaccaaagcgtggagaactcaaaacggagcttttgggactgaaagaaagaaaacacaaacctcaagtttctcaacaggaggaacttaaataactatgccaagaattctgtgaataatataagtcttaaatatgtatttcttaatttattgcatcaaactacttgtccttaagcacttagtctaatgctaactgcaagaggaggtgctcagtggatgtttagccgatacgttgaaatttaattacggtttgattgatatttcttgaaaacngccaaagcacatatcatcaaaccatttcat

SEQ ID NO: 99 - DNA, 530 bp, Artificial Sequence
  Synthetic construct NHLRC3
tgacaataactatcagatttgccttaattttgtgtttatagcatttatcaaaacgtatcctcatagactttatgcagattaatatggtcaattgatttggataaaagaaagtaatttcagggtttgtttttaagccaggacaagaagtgcaaatgcctctttgaagcaatttaggctaaactgattttgaaatttcaaaatgttttattttactttgttttattaagccaggacaagaagtgcaaatgcctctttgaagcaattcaggctaggtaaaccgattttgccatttcaaaacgttttattttactttgttttatatcagagtcttataaaacctgctgcaaatatttctgaatgtctttgtaaaagtgtttgttagtgtacctgtgattatagtacttcacttttttcctttggattaattggttaaatgaatgagaaatgtgttatgttttttactaaaaagtataaattaaaattttggaaagaaaaggcaatattatctggctccccaattaaagtttgat

SEQ ID NO: 100 - DNA, 445 bp, Artificial Sequence
  Synthetic construct SUSD3
cagagctctaagcctcagtggctcctccagctcaccccaagcccaggtgatggtgcacatggcaaaccccagacagcccctgcctgcctctgggctggccacaggaatgccacaacagcccgcagcatatgccctagggtgaccacgcagtgaggctggtgcccatgctccacactgggaggccaggctgaccccaccagccagtcagctacaactccacatcaactccacatgcgcccagctcgagactgatgagtggaatcagcttccaggtgtagggaccccttgaggggccgagctgacatccaaggctgaggaccccagtggggagtgttctgttccggcatatcctggccgtaacgatttttatagttatggactacttgaaaccactactgagggtaatttactagctgtggcctcccactaactagcattcctttaa

SEQ ID NO: 101 - DNA, 495 bp, Artificial Sequence
  Synthetic construct FAM171B
aaagcacagctgtatgtatttttgaatacatattatgatcttgagactttataaatcaatttttatgactttatgcagttgtatagggattatgccctttcagttctatagggattatgcccttttataatacataatataccacagagattacaaatgttgaggaatgaaagcacttctttgctttggcaatcattttcagaccactatgtgtttgaatcctctggtatcaatacgtattatagggttttagagatctgtgggtcaaatgatgtccctcaaaacttcctaaaaaggtgaagctcaaagtcacacattcatttataaggcgcatgagtttctcattttcccatgtacgagcattgtaaaggaattcagctgtattaatttctatttcagatctagaattgacattttgccttcttgtttccaggtgtttctattttttgtattctttcagagaaatctcatatttcggtgtatttattgctgtta

SEQ ID NO: 102 - DNA, 420 bp, Artificial Sequence
  Synthetic construct PLPP6
  misc_feature (41)..(41): n is a or t or c or g
ggactttgatctgccatacggaggttcggaacctggagaanggctgtgataagtaggttttgattgagtgaaagcatgagcttgttcagagtgaggggcatagtgaaaaaggaacagccatgcctcaaaatcaaatcatttgcattcccacagcatcctgaataccgactacctcttcacttgctaaagcagctaaactgtgaagctctaagtggtttgggtttgttgtttaaccttagcgagatcctttaactgcagcaatattcaagccagatatttggaagcaaatgatatttcctcttgcagtgtccacaaatctgaatattaggggcatgaaattaggcttaccatctgatttgtaattacaattttggaattctctgttttagttgctgaggcctgagttttctggctcttaaa

SEQ ID NO: 103 - DNA, 486 bp, Artificial Sequence
  Synthetic construct USF3
  misc_feature (26)..(26): n is a or t or c or g
  misc_feature (27)..(27): n is a or t or c or g
  misc_feature (28)..(28): n is a or t or c or g
  misc_feature (49)..(49): n is a or t or c or g
  misc_feature (56)..(56): n is a or t or c or g
  misc_feature (93)..(93): n is a or t or c or g
  misc_feature (104)..(104): n is a or t or c or g
  misc_feature (107)..(107): n is a or t or c or g
  misc_feature (108)..(108): n is a or t or c or g
  misc_feature (114)..(114): n is a or t or c or g
  misc_feature (128)..(128): n is a or t or c or g
tagtgttaagatgctgcagttgtgtnnntgtgaagaaaagattcgcttncactttnagcaggctatataaatatatatacatatatatatatnattttcacttnggnnaaaaanaaagttgaaaaatntgaggagaacataattaccttcacaaattatttatagatacactgggtgagaagcttactgtagggcagtcacccaaaaaaaactaattttagtgctttcaatagtgtaatatttgtatctattaccatggcagcagtgtctgctgcacactgtgtcctattactgtaaatctcctggtttataattcttatggaaactaatttgttttgtaatggaatccagagcagatatgtataatatgatcaggattgtcctacagttgtaaataagaataggtcctgtttattttgacatctttttacaaatgcattgtattagggtgtgaatattctgaaccatcctcttgtttaaagtttg

SEQ ID NO: 104 - DNA, 438 bp, Artificial Sequence
  Synthetic construct USF3
gttagtttcttctcaagatgcctttttggctgaacattaccatttttctttccccagggtttgaaagaaaagggccttttttggatttaactatattctgtctgcattctattcttcatgcgtaggtttctactagaacctgtggaggggggacaccaaatttagattgagtcagtccatgctgataagaagcgtgaggaccttgttggacacagagcttctagatctttccacctgtagcctctaccctgttcctgcactttacctgaaactgtgcctctggtctttagaccagctgttggaacatatcactcacccactcctctgttatagcaaagggccagctgggatggtacacgctgtgtaatcagacaccttctcgacaagtacagctgatgtttctcactgttgccttaccattctctgagccatctttga

SEQ ID NO: 105 - DNA, 456 bp, Artificial Sequence
  Synthetic construct RDH10
tgcctgcactcaaatagccgatgttactgtccctagattagagacttgattaagggcttgtttgtaccaaaagtggggaaacaatgccatgacctgtgttttagtttggctgcaccacagatcaaatctgcactgtgtctacatataggaaaggtcctggtgtgtgctaatgttcccaatgcaggacttgaggaagagctctgttatatgtttccatttctctttatcaaagataaccaaaccttatggcccttataacaatggaggcactggctgcctcttaattttcaatcatggacctaaagaagtactctgaagggtctcaacaatgccaggtggggacagatatactcagagattatccaggtctgcctcccagcgagcctggagtacaccagaccctcctagagaaatctgttataatttaacaacccacttatccaccttaaaactgag

SEQ ID NO: 106 - DNA, 487 bp, Artificial Sequence
  Synthetic construct POP4
  misc_feature (219)..(219): n is a or t or c or g
ttctgaggcagctcaagtgctctcccccaagcagcacgcagtcaccgctgccagtgatgaacacacgcacgggctaggcacatggagcccaagcacactgccacctcggacacgggccccggcccagcgactgccttgtggtgtggccgtcctcatctgccacaggaagacagacttcgagggacctcttgcctaaggcctcgctgactcaagggtagnattgagatttggacctaataggctccagaactgtgctttgagctacagggtggctgcaggatgagacggatgcagttcaaagtgcaggatgtgctgcctttattacctgctttcccgctgagcaaatccctgtcttgagtgggaagtggcatctattcccccagccatctgaaaatgctgtgttcataaccaaataggtgctgtagcttcctagccatcccgctcttacccgcccctctgcaatgtaaaaggcgtccactgttgtgta

SEQ ID NO: 107 - DNA, 577 bp, Artificial Sequence
  Synthetic construct MVB12A
  misc_feature (63)..(63): n is a or t or c or g
  misc_feature (86)..(86): n is a or t or c or g
  misc_feature (89)..(89): n is a or t or c or g
  misc_feature (95)..(95): n is a or t or c or g
  misc_feature (118)..(118): n is a or t or c or g
  misc_feature (158)..(158): n is a or t or c or g
  misc_feature (213)..(213): n is a or t or c or g
  misc_feature (338)..(338): n is a or t or c or g
  misc_feature (339)..(339): n is a or t or c or g
  misc_feature (443)..(443): n is a or t or c or g
  misc_feature (460)..(460): n is a or t or c or g
ggcatccactctgcggaggaatgactccatctacgaggcctccagcctctatggcatctcagncatggatggggttcccttcacantcnacccangatttgagggcaagagctgcagncccctggccttctctgcttttggggacctgaccatcaagnctctggcggacattgaggaggagtataactacggcttcgtggtggagaagaccgnggctgcccgcctgccccccagcgtctcatagtccctcacccttccgcggaaagagcccccttactccacctccccgccagcctggggccaccccccctcactgcatcctggggccacccccactnnactgcatcctgggaaccttcgccctgcaaggcgtttgctatcttcagccactgggcggagctgcagccctggaggagggggcgggtcgaggctgcgtggtgatngggtctccgcccccacnccctgccgggcagggctggagctggacagaagccagtgcctttaagtcatttgtgtcaaaaccctctggggtccggaggctgtgcgggtgtcctcctggcaataaacactacccggt

SEQ ID NO: 108 - DNA, 335 bp, Artificial Sequence
  Synthetic construct LOC100506990
  misc_feature (63)..(63): n is a or t or c or g
  misc_feature (303)..(303): n is a or t or c or g
acacccataaaagatctgtgctgaggtggattagtcaaagaggaaagacttgcagttgagatngaggaaggccactgtctcctgactgcccctgggaactgaatgtctcggtataaaacacgattgtacatttgttcagttctgagatgggagaaaaaccgccctatggtgggaggcgagacatgtttacagcaatgctgccttgttatcctttactccactgagatgtctgggtggagagaaacataaatctggcttacatgcacgtccagtcatagtaccttcccttgaacttcattatgncatagattctattgctcacgtttgttgctgac

SEQ ID NO: 109 - DNA, 141 bp, Artificial Sequence
  Synthetic construct MYLIP
tctgcaggtcgcgtgtggagcatgtccagcacgtctatctgccaacgcacaccagtcttctcaatctgactgtaatctaatctgttgtgcttttgttggacttggcatgtttccatgaactgcactattataaactattaa

SEQ ID NO: 110 - DNA, 542 bp, Artificial Sequence
  Synthetic construct DNLZ
  misc_feature (40)..(40): n is a or t or c or g
  misc_feature (74)..(74): n is a or t or c or g
  misc_feature (85)..(85): n is a or t or c or g
  misc_feature (91)..(91): n is a or t or c or g
  misc_feature (100)..(100): n is a or t or c or g
  misc_feature (104)..(104): n is a or t or c or g
cccgtccagaggtcgcggggaggcggcgggcctgggcctngggctggcggcgctcaagctccgagcaggggccngggcccgcggnggctcntggggcgcngtgnaggcggcgcactaccagctcgtctacacctgcaaggtctgcgggactaggtcctccaagcgcatctccaagctggcctatcaccaaggcgtggtcattgtgacctgccccggctgccagaaccaccatatcatcgctgacaacctgggctggttctcggacctgaatgggaagagaaatatcgaagagatcctgacggccagaggcgagcaggtgcaccgtgtggcgggcgagggggccctggaactggttctggaggctgcaggggcccccacatccactgcagctccggaagcgggtgaggatgagggtccccccagccctggcaagacggagccgagctgactcctgcgctccccggcactgtgggacttcggccttccagaaggaggttttggccctgggcctctctggacgtggcctgttttctatcaataaa

SEQ ID NO: 111 - DNA, 428 bp, Artificial Sequence
  Synthetic construct KLKB1
ggaaattggtattatctctgttttatgcacttacatttatcccttacattttgtttttagtgaccctacatgacattaaatttaaagtaaaacattgtttaatgttaccttttggcttgagaatgtctttcagctccagaattattgttactcatattttaatcagtaagtcatttaagctatgacagagtaggaattgagaaattatttcatatgctacagtattgaaatgtggatgctgccttgttttataagaagatgatcaaggtttgtgtgcccattacctttcctctgcctgaaagacgtgtctcaagaaaaataaattctattttagatgcaggtactgcattttattctaagaattgatatcaattcaaaacatagaaaactgtaaaagataaatcaggagatggctgattcataatggg

SEQ ID NO: 112 - DNA, 301 bp, Artificial Sequence
  Synthetic construct ZNF302
cacacagtttcttcataacctcagaaagcctatatctttgtcagctataaagagcatttactttgtttttactgcagttgtttaatatagtagtatcaattctgtattttaagcttgataatccagcagcaaattgagattaatggaaggttggaaaaaaaggctatgaaacaaaatgcaacctttcccacatgaatataggcacaaaatgatgatttgtaatcctgaatgccttccacatcttatttctgatactttccttttttccatactattttatccatttcgtacagcttaccca

SEQ ID NO: 113 - DNA, 136 bp, Artificial Sequence
  Synthetic construct ZNF302
ctgcttctgtgaactttggattctctgctctatttgagcattgttcagaagtcactgaaatatttgagttgtcagaactatgtgttttctgggtgcttcatttcttatccaattctcctaattccactgtagaagc

SEQ ID NO: 114 - DNA, 483 bp, Artificial Sequence
  Synthetic construct RAP1A
  misc_feature (67)..(67): n is a or t or c or g
taaaatatcccctcttgaatcagctccaggcttaaaagggcagagtggtttggattcttgtccaggntctggaaagagctcatcttgccgagagggaaataaatgtgagctttgaggcggtctaactacccagcttttagtatttatgttccccaaaatgtaaaaactgtaagttattcttgaaaatgtagtccagacatttctttgatcctggattttactgcagtttcatcatgggtttgttgaatgtgcagtccatttattgccctgtcttgcttaaatattcttgttgagaacagctggaggctttgactgggttcctagttctgttttctgggaaggcaacgtggattctgttttgtagacattaatgagctttaaatgggaattggggatgatgtatgttcgttgggcatgctgagaagcggcctcctgtggttctgggcccaaggtgtgacatacatttcccactaattgcttctc

SEQ ID NO: 115 - DNA, 443 bp, Artificial Sequence
  Synthetic construct CD59
  misc_feature (68)..(68): n is a or t or c or g
  misc_feature (280)..(280): n is a or t or c or g
gcaggggcgttaggaagctccaacaggatggtacttaatggggcatttgagtggagaggtaggtgacntagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatncttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatac

SEQ ID NO: 116 - DNA, 424 bp, Artificial Sequence
  Synthetic construct SNX20
gatggactgggatgcccttaactgaggtctgctgtctggaaaggacaccacaggcccagaatgaaggggcatctggaccaatgacctagattcaacacagggcttggagctggtggtacaggctggttttggcccaatgtgtgttggaggctggaagcccctgaagacctaaactgcctggatgagcttattagggtgaattaatgagtctgtaatttgggaaccttaatgtgagacaccacttaaggagaggaacttggcccagcaagtgatcactctagaagtggacagctatgcactagactgcaccagaatctgttcaacagcgacaccttgtggtaaaaactgtagacgccccatcaggctggatgggaaactagaaacagactgtctcatctttggacagatatccctcaaatctccc

SEQ ID NO: 117 - DNA, 430 bp, Artificial Sequence
  Synthetic construct SEMA4D
aggctctgaaggctgatcctacaccgtcccagtgactccccttgacagagtgcccccaccccctaatagccaacagggttagcatggccagcacagatcgctgcttttattgatgcaaatcaagcctgctgcttctcctccctgcagacttagccaaggaactccaagatgcatgactgggacaagaaaaggtgagactccacatggaaatgccttgccctaaaccttgaatgactgtgagatgcgatctgggagtgcatctgtcaagtctttgtgttttcttcactaacctcagaatactgggctctattttatcaagcgctgcagtttatgcctctgtcccgtcaatgctcagcttctgcaacaggacaccaaacttgatgcagaaagccaaataggtcaattatgcaaatctcctggtgccatatta

SEQ ID NO: 118 - DNA, 442 bp, Artificial Sequence
  Synthetic construct ZNF224
  misc_feature (384)..(384): n is a or t or c or g
gacagcttttgtcaaccttcttatccaatattcttccaccaacgctatgaaacaggaagagaaaatggagtttggtaatttcatcaaatatattcattattgggggcaggagtaacatttaaatcctcctctttattgctggccgtaaaaatatgtcttttggagagcagaacatgccagaatgtataaggtgcaactgtttgatatgaaaaccataggataggaaactatgcctatgacactaaaactccaaaaagtagaagaccacattgatgatgtcactgcattttaaaatggaaagaacaagtgttcaagaacaggaatttgtgtgaattgcatttattcctataataatatgaactcaatgagatgatgacatagacncaataacaaaaggagaaaaacaatgtggctttttttagccttcataacctaatcaaat

SEQ ID NO: 119 - DNA, 416 bp, Artificial Sequence
  Synthetic construct RNASEL
aatcctttaaggtcaacattgttatccttattttgctgatgaggaaactaaggttagaaacattttgatttcctctaggacgtatagctaggaagtgttactatcttgatttgaacaaattttctggtgctaagtctgatgttctttccatgaatcattgtggtggttgagatggagctttgtaatgggaataaaacagtaccttaggttctttctgaaaaggaggtatctagcaatggataaatagataccactgaatgaaattaaatgttgattaggaacaaatttaaggcttaaaaaatactttatgagcagcaagattgctttaacttttaaaatgaagctttggttctctgatttgtaatgagcacctggatatgtcaattaaaatgcccatttgtgaagcttactcaata

SEQ ID NO: 120 - DNA, 448 bp, Artificial Sequence
  Synthetic construct ARSD
atactggggatgggaggaccttgcactattcccctcagtccatctatcgaggtctttgcaggaagcatactgggaattgaaacgagagcctaaatgacatctaagaaaggcagtgttcaataccaggtattaggtgaggatgggattctaaggacatcagtgggaggcagggagccaccttcagacctcagcatggaagcttccaagatccagaggaagaggcaacagcactgagagtcataggtagaagaatcatcacagccctgctaaccaggcagctgatgcccctctcccctggctccctgtgtccaaatcctacaggggcatctgttggctgaactcaacctgaagccaaagagaagatgagtggagagaggcaacatttatagagctcaggtttctagggctggagagggatctggagggacacacaggagacacctggcat

SEQ ID NO: 121 - DNA, 330 bp, Artificial Sequence
  Synthetic construct SLFN5
ggtaatagtgtttgactccagggaagaacagatgggtgccagagtgaaaaaaagatagcttttgctttttatgacttttggattctgtaccacgtaataattttgatgtaaattttgctgtgtgtgtttttacttgttcatgtagtgattttataaattactcttttaattttctatcaatgaatatcctgggataaacccctcatgatcataatgaataatgatgtgtggagagtggggagggtttacatatgaaaaatgtagaaaatacaaaaagtgtctatatatacaaaaatgtaagtgttaacatttttatatttgcttcaagct

SEQ ID NO: 122 - DNA, 396 bp, Artificial Sequence
  Synthetic construct ABCG1
  misc_feature (317)..(317): n is a or t or c or g
gtaactctaatcagcgtgcaaaggcgtgcatatatatctgctatgtaattatataatgtttttataatgctgtgtggataacttttcactgatttagttacttgactgtaggaaacactgttgctttgcacgctgtgagaggtttgcactgttacagatgaaggcaacatatttacatatgtacatgtatatacatatatatatattcaaaaataacatcggctctttggggtgtctgtttccttggttgtctcacgccactggaacattgaacccagacagaccctaggatgtgaggttcctgactgcaccttacngaccacagagccacgtgtctatttctgtagacctatctccttagatttgtacatgcattttttgcggacttgaactttg

SEQ ID NO: 123 - DNA, 480 bp, Artificial Sequence
  Synthetic construct TTC39B
gtgttagtaacatccatctgctgatcaaggaggcattggatctggtactagaaggtgaaattgattgtagctatcaaagcattttatcaatgtaagtcaagaaaaaagaagaaaactgtgaacctctgatatttttaacataaaaactgttcccaatgagtgttctcttgctgattttgtgttaatgttattgtctatgatttttaagctaatgctaatataaaatctaaaatttcaacatgatgacaacaattcctgtagcctgtttttaccattaggatgtttttgaaaacagatgtcatcttagaaattatatttttaagtgcaaataaatcatcctgacttgaaagtcaacacattttatttttcattccgtagtatcacagaatatgctgcatttagatacaggtttaatttgccagattttctcaaaattctgtatttttatattgctacaactggtttacttaacatgcaatt

SEQ ID NO: 124 - DNA, 414 bp, Artificial Sequence
  Synthetic construct ABCG1
gtgtctacctacacacaagcacatgtgcacacatgcacacacatgcatgcaagcacacgcacacacacacatgcacacacactcacatggacacacacacacacacatgcacacacatacatgcacacacatgcacatggacacacatacatgcacacacatacatgcaggtgtgcagacttggctccaggcgtgtgtttgatagtattattctatgatatttccctcatctccatagaataccagcttctgaatcctcaatcagcctttactgcaagaagaaaagaaaaacctctctcattccaggtctgtggtgcagatgggaagagtatagtcaaaacccattaaggccttagtcaaatgccagccgaattagaacgcaatgaacgttagacaaaacaacccaactggcca

SEQ ID NO: 125 - DNA, 463 bp, Artificial Sequence
  Synthetic construct PTEN
  misc_feature (38)..(38): n is a or t or c or g
  misc_feature (44)..(44): n is a or t or c or g
  misc_feature (51)..(51): n is a or t or c or g
  misc_feature (57)..(57): n is a or t or c or g
  misc_feature (62)..(62): n is a or t or c or g
  misc_feature (78)..(78): n is a or t or c or g
  misc_feature (79)..(79): n is a or t or c or g
  misc_feature (100)..(100): n is a or t or c or g
gatatttctagtctatgtgtttcatctgcaggtttttncaaatngttgaantctctnaaaanttttattgaaaaaaannccatatatgtaagtggacccnacacatttcaaacctgtgttcaagggtcagctgtgtaaataattttcctcaaaattaaagtggaaaaggagagttactactagtagaaagtagaactgtacccttgggcaggggtgtgtgtgtgtagttaaagatcaatttaacttaaaaggtcttggttagagaataaaactggcccttattagctttaatttacatgaaaaatgaaaaattttaggccaggcacagtggctcaggcctgtaatcccttaactttgggagaccaaggggagtggatcacttgaggtcaggagttcaagacagcctggccaacatgctgactcacccttccctactgaaaatacaaaagttagccaggcgcag

SEQ ID NO: 126 - DNA, 455 bp, Artificial Sequence
  Synthetic construct ZNF81
acattctcccacatacctggaatatcattatcaatctaattctatgatatatccagtccatttccaaatttctcccattgtccccaaacatattttacagttttaatgaggtagaattagcatacagaaagtggaacttgtttaaagtgtacaatttgatgagttttgacgtgtgcacctttccaaccatcatgacaatacaaataatgaacatatcaatcaccccaaaaaagtttccttatgcctctttgtcattcagccccctttccccacattcttcccacatccacaggcaatcatggatctgttttctattgttatagattagtttgcgtttcctacaattttatacacatggaataatactataggtactcttgtttggctccttccatccagcataataagtttgagattcattcatgttgttatatgcgtctatgttcattcctttt

SEQ ID NO: 127 - DNA, 530 bp, Artificial Sequence
  Synthetic construct DENR
gctcaaaaattctcctgtggtgcctcagtaacaggggaggatgaaattatcattcagggagattttacagatgacataattgatgtcattcaggaaaaatggccagaggtagatgatgacagcatcgaagatcttggagaagtaaagaagtgaatttgaaaatttgtctgtatttaatggcctgaactgagagttgatatggccaaagggagagaggccttttaaaatatatatatatatacacatatatatgtatatatacacatatatgtatgtatacacatatacacatgtatatatacatgtgtgtatgtatgcatgtatatacatatatacatacacatatatgtatacatatatacacatatatgtatacatatatacacatatatgtatacatatatatatattctacagtaaaactgtagactgtcctcgtccttggcattttcactgttctgtacaaggctgcttgtttttttattgccaaagtcaaataaacgggagactgtcatgctcatgcatgaata

SEQ ID NO: 128 - DNA, 185 bp, Artificial Sequence
  Synthetic construct MTFMT
gtaatataccacaaacagtcacaaatactattggtttattgcaaggatggttggattggtgttcgatcagtgatgctcaagaaatcactaacagctactgacttctacaatggatatttgcacccctggtaccagaaaaattcccaagctcaaccaagccaatgcagatttcagactctcagact

SEQ ID NO: 129 - DNA, 423 bp, Artificial Sequence
  Synthetic construct nd
tgatgtggacaccagtacccagtaaattactgtgaaaaaaaggcaacttctcttggtccagtttacctttagataaatgttttaagggtaaaaatatgtgtatttcaagatatgtcttatgtaaacttttttaagttatgaaaatcatccatattgaaaaatagaatgttttctattttgaaactgttttgcattgtcagttacttaagcagccattttacattttggtttttgagaacataatagggacctgtatactccttgtggggccgcattttaagactcctgacagctcatcctgcaaaattaaaatccaaaatctaagtcgcaaaattctcttttctccttcctcagttatttacttagagtgtagaacagacatcctcttagagttttttgtaactatcagttatcgttaacatc

SEQ ID NO: 130 - DNA, 345 bp, Artificial Sequence
  Synthetic construct ATP5S
aaaacctaagcatagcccactagtttttactgtttcctttattatattgcaatctgcaaaactatttgtattcagccaagtatctatgtccagttttaataaaatagtagcctaagcttacaaaatagacaaattaagcagcaactgttgattctgagtaagtctgtatgctatcaagaacacaagaatgcaggatcatgggaaatgcacgcaaatcctgttgaagcaatgcatgcttcaacaaccactgaccactttgcataattgactccatcaatgaatctgtaatttagcttctaatttttcataattgtgcatttatggtagtatgcttcactaacttta

SEQ ID NO: 131 - DNA, 374 bp, Artificial Sequence
  Synthetic construct ZNF818P
  misc_feature (255)..(255): n is a or t or c or g
  misc_feature (330)..(330): n is a or t or c or g
gggcctacttcattaggtgtggttcattcctattcatgctccctggaagaacaagaatgctgaattttgaaatttaatattgtatgaattagcatcagggagaggtggagaaaaatacaaaactaaaagtcatgcttattgtgttcagtgtgcccttctccagagggccactggcttataggaaaggattgctgctctaccagttgaccaggagatggcacgccaggacattaagacactggagttttgtttcgnttttttttttttttttgagactctgcctcaaaaaaaaaccaaaccaaaccaaagaaccagaatagcatgtgcacntatacacagacgtttcacaactggcattatgttttgctactgtt

SEQ ID NO: 132 - DNA, 501 bp, Artificial Sequence
  Synthetic construct ZNF829
  misc_feature (193)..(193): n is a or t or c or g
  misc_feature (214)..(214): n is a or t or c or g
aaatcctatttagtacaaggtacattatttaggtaatgagtccattaaaagccaacactttccccactacactatatgtgtatgtaacacaactgcccttgtaacttcctaaacctataattaagaaacaataaaaggcaaattaagaatgcttttttaaaaggtgggggcattatgctaataagttactgtnggatttcagagtgcagagtangaaagatcacaagaatttagtgtggtaggtgggaacagaaaatgggtgtataaattttattgacgtgggagtactggatattgtagagacagatatcatcagggcaaggagattaaagatttttgcattgacggtttgacactatattgtggtaataacactgtatgtgttgggagatagaacaggaaacatcttccctggaatatgtatactattaaatgttttatcaaacttttgatcaaacaagacagcacaatttataatttcatttctatttctatgttatg

SEQ ID NO: 133 - DNA, 472 bp, Artificial Sequence
  Synthetic construct CSKMT
  misc_feature (80)..(80): n is a or t or c or g
  misc_feature (82)..(82): n is a or t or c or g
  misc_feature (83)..(83): n is a or t or c or g
  misc_feature (85)..(85): n is a or t or c or g
  misc_feature (86)..(86): n is a or t or c or g
  misc_feature (89)..(89): n is a or t or c or g
  misc_feature (90)..(90): n is a or t or c or g
  misc_feature (91)..(91): n is a or t or c or g
  misc_feature (92)..(92): n is a or t or c or g
  misc_feature (93)..(93): n is a or t or c or g
  misc_feature (94)..(94): n is a or t or c or g
  misc_feature (95)..(95): n is a or t or c or g
  misc_feature (97)..(97): n is a or t or c or g
  misc_feature (98)..(98): n is a or t or c or g
  misc_feature (100)..(100): n is a or t or c or g
  misc_feature (102)..(102): n is a or t or c or g
  misc_feature (103)..(103): n is a or t or c or g
  misc_feature (104)..(104): n is a or t or c or g
  misc_feature (106)..(106): n is a or t or c or g
  misc_feature (107)..(107): n is a or t or c or g
  misc_feature (108)..(108): n is a or t or c or g
  misc_feature (109)..(109): n is a or t or c or g
  misc_feature (110)..(110): n is a or t or c or g
  misc_feature (114)..(114): n is a or t or c or g
  misc_feature (117)..(117): n is a or t or c or g
  misc_feature (118)..(118): n is a or t or c or g
  misc_feature (119)..(119): n is a or t or c or g
  misc_feature (121)..(121): n is a or t or c or g
  misc_feature (122)..(122): n is a or t or c or g
  misc_feature (123)..(123): n is a or t or c or g
  misc_feature (124)..(124): n is a or t or c or g
  misc_feature (126)..(126): n is a or t or c or g
  misc_feature (127)..(127): n is a or t or c or g
  misc_feature (128)..(128): n is a or t or c or g
  misc_feature (129)..(129): n is a or t or c or g
  misc_feature (150)..(150): n is a or t or c or g
  misc_feature (156)..(156): n is a or t or c or g
  misc_feature (168)..(168): n is a or t or c or g
ctgccagtcctttgcaagtgctggatgtgggctgtgggacttccagcctatgtacaggcctttacaccaaatctccacancnngnngannnnnnngnngngnnntnnnnncctntgnnngnnnncnnnntgaatagcctcctggagggtngcccangccaaacacctntatgccctggacaccctgcctcaagcctccacttcatgcacgccgatgctcagaacctgggggctgtggcttcttcaggctctttccaactactgctggacaaaggcacatgggatgctgttgcccggggaggtctgcctagggcttaccagcttctatcagaatgcttgagggttctaaaccctcaggggaccctgattcagttctcagatgaggaccctgatgtgcgactgccctgcctggaacaagggtcctatggctggactgtgactgtgcaggagctaggcccgttcaggggcatcac

SEQ ID NO: 134 - DNA, 371 bp, Artificial Sequence
  Synthetic construct RGS3
atgtatctgactttctataacccccttccttggggcctccactcatgaggcatgggaaggtgttttttttctttgtttgtttgtttgtttgaatgcagttgactttttttttgttgtattgttgcaaaagtaatccatgtgattgatgtttattatgaagaaaacatttaatacagatgaaccaggagaagaaaatgaagttcatccttaatcccagcacccagagataaaccactgttaacattttggaatgtgtcctgtcaggcattcgtttatacacacatatatcttatttgcaaattggggttactggtgcaatttttctaacctgctttttctcccccatttaacagcgtagtgtatccatgttt

SEQ ID NO: 135 - DNA, 396 bp, Artificial Sequence
  Synthetic construct nd
  misc_feature (285)..(285): n is a or t or c or g
ataagttctttacggtcatgttctaataaatttgaaaatctgaatgaaatggactgttctctaggaaaagatcaaaattggcttaagaaatctgcactagcaaaccgttgcagtcttttatttatttatttaattttctgtagagacagggttttgccatgttgcctggactggtcttgaactcctgagctcgagcagtcttcctgcctcagccttccagaatgctgggattacaagcatgaaccaatgtgcctagcctacagtcttttataaaattatcaaagnattcttttttaaaatggctgcaagtccaggtgttttacaagtggattgttttaaaatttcaacatgaaatatttaaagttatttaaactgtcccagaaggtagaaaaatca

SEQ ID NO: 136 - DNA, 453 bp, Artificial Sequence
  Synthetic construct nd
  misc_feature (170)..(170): n is a or t or c or g
  misc_feature (221)..(221): n is a or t or c or g
  misc_feature (225)..(225): n is a or t or c or g
  misc_feature (226)..(226): n is a or t or c or g
  misc_feature (227)..(227): n is a or t or c or g
  misc_feature (229)..(229): n is a or t or c or g
  misc_feature (232)..(232): n is a or t or c or g
  misc_feature (233)..(233): n is a or t or c or g
  misc_feature (234)..(234): n is a or t or c or g
  misc_feature (235)..(235): n is a or t or c or g
  misc_feature (394)..(394): n is a or t or c or g
aggaaatcatcggcacactgaaccccctactcagaagtttacctatgaaacaatcttgcacatgtatgcttcaaaaacaaataacagttagggaagaaagagagagagagagaaagagagagagacaagtaaaataaagcaccacctccttgacctgactcagggcgttnggggtcttctggggaaatgttctgaaacaatggagtattttggtctgttcnttcnnntntcnnnntttttttttttttaagacggactctcgctcagccacccaggctggaatgcagtggtgcactgggttcactgcagcaaatatctcccgggttgaagcgattctccagtctcatcctcctgagtggccgggattacagtcacgcgccataatgccctgctnatttttgaacattagtagagaaggggtattgccatgcttgcgacgctggacttgaaggc

SEQ ID NO: 137 - DNA, 424 bp, Artificial Sequence
  Synthetic construct CECR7
  misc_feature (233)..(233): n is a or t or c or g
gacagcgccaaggcagcaactgggatgggagagtgatctgctgccctctcctggcaatgtgaggaattgtcctaagcacagacaactccctccctgccttgcacaagtgcaggatgggggaaactggaaaaggagctgatggaggattggggaggtggatgctgtctaaggagacaaactgaattgaggggttgctgggactctgccggctcacacgctattcccatgtcatnggcccaggggccaagtcagtgcttccaaaggagggtgcatcctgagagaggctctggctcccccaacaaaaccctaaactcttgaaggacatcttccttctcagtataccctattgctgtgttgttgtaactatcactgtaacttttattcacttcagtgggcgatcactgagtttttcctccattgttgt

SEQ ID NO: 138 - DNA, 478 bp, Artificial Sequence
  Synthetic construct ARHGAP30
  misc_feature (79)..(79): n is a or t or c or g
  misc_feature (322)..(322): n is a or t or c or g
ttggatatgtcctgttattcgcgtcagcagtcactcatccaaagaccccaaagcttctagtgttctaaaggctcacacnttctgtgacaggaatcttgattcccattcttctcaatcaagacctaccctgaatggatttggagtagtgtggtataatgggaaaaattcaagcttgggggccaggcagatccaggtttcaaacccatctctgccacttcttacctctgagacctttggcaaacattttgacttaattgagccttagtttcctcatctataaactggagatactagcaccaaccttccagaattgtgtggattnaataaaatcaaggacatagattttgtgagcacaggatacttagtccagttcctggcacataatgaacgcattatgtaaatataagtttctcttccttatttcctgaccaccatgaacctagctctagtttctggccactagtctccatgacaacaa

SEQ ID NO: 139 - DNA, 320 bp, Artificial Sequence
  Synthetic construct nd
  misc_feature (94)..(94): n is a or t or c or g
  misc_feature (95)..(95): n is a or t or c or g
gaaatggcacattttctggatgtgagagttggtcaaaagatcacaaaaaaagtcaaaaaataattctactctgtgaatgaaaaatggatatttnngtacttaccctcataagcattaaaagaaaataatgcatgaaattccatagaaatgtgcctatcatgttatactgactcaaaccagaagacctagagtatgatattgctaatataatacatgtggtgggtatgagtggaagtatgtgtgtgagatttatcattgccatagtgtaaaagagttgaattagcttccacttgactagatgagagctcttagttcttatt

SEQ ID NO: 140 - DNA, 271 bp, Artificial Sequence
  Synthetic construct ZNF667
aaagacactgtgtacaacgttggacactgtgcaggatgatgccacttcatcttggatgctaatctgccatgttgacttctgattaaccccaggcccaggaatgcctcaagatttctactttacttactgttgcttgtgtaagccaagacaaccttgatgttatcataaacatgtacttacctaagtcctgtcctttggcaaattatgggctatgagacacagcattcttgcctttccctgaggggtcaatttcagcgatcctacacattcc